(19) United States
(12) Patent Application Publication
(10) Pub. No.: US 2011/0113508 A1
(43) Pub. Date: May 12, 2011

(54) MODULATING PLANT CAROTENOID LEVELS

(75) Inventors: Steven Craig Bobzin, Malibu, CA (US); Joon-Hyun Park, Oak Park, CA (US); Boris Jankowski, Newbury Park, CA (US); Karen Chiang, Houston, TX (US); Amr Saad Ragab, New Haven, CT (US)

(73) Assignee: CERES, INC., Thousand Oaks, CA (US)

(21) Appl. No.: 12/377,778

(22) PCT Filed: Aug. 20, 2007
(86) PCT No.: PCT/US07/18519

§ 371 Date: Dec. 8, 2010

Related U.S. Application Data
(60) Provisional application No. 60/838,646, filed on Aug. 18, 2006.

(51) Int. Cl. C12N 15/82; C12N 15/29

(52) U.S. Cl. 800/278; 536/23.6

(57) ABSTRACT

Methods and materials for modulating (e.g., increasing or decreasing) carotenoid levels in plants are disclosed. For example, nucleic acids encoding carotenoid-modulating polypeptides are disclosed as well as methods for using such nucleic acids to transform plant cells. Also disclosed are plants having increased carotenoid levels and plant products produced from plants having increased carotenoid levels.

### CROSS-REFERENCE TO RELATED APPLICATIONS

This application claims priority to U.S. Provisional Application No. 60/838,646, filed Aug. 18, 2006, the contents of which are hereby incorporated by reference in their entirety.

### TECHNICAL FIELD

This invention relates to methods and materials involved in modulating (e.g., increasing or decreasing) carotenoid levels in plants. For example, this document provides plants having increased carotenoid levels as well as materials and methods for making plants and plant products having increased carotenoid levels.

### INCORPORATION-BY-REFERENCE & TEXT

The material on the accompanying file contained in the attached compact disc hereby incorporated by reference into this application. The accompanying compact discs being submitted in quadruplicate contain one identical file, 179WO1-sequence.txt, which was created on Aug. 20, 2007. The file named 179WO1-sequence.txt is 714 KB. The file can be accessed using Microsoft Word on a computer that uses Windows OS.

### BACKGROUND

Carotenoids are a widely distributed group of naturally occurring pigments, usually red, orange or yellow in color. Carotenoids are an important class of metabolites distinguished by a broad range of structural diversity, physiological function, and biological activity. Carotenoids play critical roles in many normal cellular and developmental processes in both plants and animals. Carotenoids also have significant pharmaceutical and neutraceutical applications. In both natural and synthetic forms, carotenoids have been shown to have antioxidant, anticarcinogenic, anti-inflammatory, immunomodulatory, and cardioprotective effects. Carotenoids and degradation products of carotenoids also contribute both to the flavoring and aroma of foods, as well as to the aroma of flowers.

In humans, dietary carotenoids such as α- and β-carotene are a major source of vitamin A precursors. Deficiency of vitamin A is a major cause of blindness and premature death in developing nations, particularly among children. Other carotenoids, for example, lycopene, lutein and zeaxanthin play an important role in human health by acting as biological antioxidants. The antioxidant effects of carotenoids are believed to protect against degenerative eye disease and certain types of cancers.

Plants can serve as natural sources of carotenoid molecules. In light of the wide variety of useful applications of these molecules, it is desirable to produce plants having modulated levels of carotenoids. One strategy to modulate plant carotenoid levels relies upon traditional plant breeding methods. Another approach involves genetic manipulation of plant characteristics through the introduction of exogenous nucleic acids conferring a desirable trait.

### SUMMARY

Carotenoids are a diverse class of metabolites derived from isoprenoid units. Carotenoids are generally divided into two classes: the carotenes, which are unoxidized, and the xanthophylls, in which some of the double bonds have been oxidized. Useful carotenoids include molecules from both classes, for example, α-, β-, and ζ-carotenes and xanthophylls, e.g. lutein and zeaxanthin.

The invention features methods and materials related to modulating (e.g., increasing or decreasing) carotenoid levels in plants. The methods can include transforming a plant cell with a nucleic acid encoding a carotenoid-modulating polypeptide, wherein expression of the polypeptide results in a modulated level of one or more carotenoids. Plant cells produced using such methods can be grown to produce plants having an increased or decreased carotenoid content. Such plants may be used to produce, for example, foodstuffs and animal feed having an increased nutritional content, and/or modified appearance or color, which may benefit both food producers and consumers, or can be used as sources from which to extract one or more carotenoids.

Thus, in one aspect, the invention features a method of modulating the level of a carotenoid in a plant. The method can comprise introducing an exogenous nucleic acid into a plant cell. The exogenous nucleic acid comprises a regulatory region operably linked to a nucleotide sequence encoding a polypeptide, in which the HMM bit score of the amino acid sequence of the polypeptide is greater than 50, the HMM based on the amino acid sequences depicted in one of FIGS. 1-9. A tissue of a plant produced from the plant cell has a difference in the level of a carotenoid as compared to the corresponding level in tissue of a control plant that does not comprise the exogenous nucleic acid.

In some embodiments, the method comprises introducing into a plant cell an exogenous nucleic acid comprising a regulatory region operably linked to a nucleotide sequence encoding a polypeptide having 80 percent or greater sequence identity to an amino acid sequence selected from the group consisting of SEQ ID NOs: 80-87, SEQ ID NO: 89, SEQ ID NOs: 91-93, SEQ ID NO: 95, SEQ ID NO: 96, SEQ ID NO: 98; SEQ ID NO: 99, SEQ ID NO: 100, SEQ ID NO:101, SEQ ID NO:102, SEQ ID NO: 104, SEQ ID NO:106, SEQ ID NO:108, SEQ ID NO: 110, SEQ ID NO: 128, SEQ ID NO: 129, SEQ ID NO: 131; SEQ ID NO: 133, SEQ ID NO:135, SEQ ID NO:137, SEQ ID NO:139, SEQ ID NO:141, SEQ ID NO:143, SEQ ID NO:144, SEQ ID NO:146, SEQ ID NO:148, SEQ ID NO:150, SEQ ID NO:152, SEQ ID NO:154, SEQ ID NO:156, SEQ ID NO:157; SEQ ID NO: 159, SEQ ID NO: 161, SEQ ID NO: 163, SEQ ID NO: 165, SEQ ID NO: 167, SEQ ID NO: 169, SEQ ID NO: 170, SEQ ID NO: 172, SEQ ID NO: 174, SEQ ID NO: 176, SEQ ID NO: 178, SEQ ID NO: SEQ ID NO: 180, SEQ ID NO: 182, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 196, SEQ ID NO: 198, SEQ ID NOs: 199-203, SEQ ID NO: 205, SEQ ID NO: 206, SEQ ID NO: 208, SEQ ID NO: 210, SEQ ID NO: 212, SEQ ID NO: 213, SEQ ID NO: 215, SEQ ID NO: 216, SEQ ID NO: 218, SEQ ID NO: 220, SEQ ID NO: 222, SEQ ID NO: 223, SEQ ID NO: 224, SEQ ID NO:226, SEQ ID NO:228, SEQ ID NO:229, SEQ ID NO:231, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-257, SEQ ID NO:259, SEQ ID NO:261, SEQ ID NO:262, SEQ ID NO:264, SEQ ID NO:265, SEQ ID NO:267, SEQ ID NO:269, SEQ ID NO:271, SEQ ID NO:273, SEQ ID NO:275, SEQ ID NO:277, SEQ ID NO:279, SEQ ID NOs:281-290, SEQ ID NO:292, SEQ ID NO:294, SEQ ID NO:296, SEQ ID NO:298, SEQ ID NO:300, SEQ ID NO:302, SEQ ID NOs:304-313, SEQ ID NO:315, SEQ ID NO:316, SEQ ID NO:318, SEQ ID NO:320, SEQ ID NO:322, SEQ ID NO:324, SEQ ID NO:326, SEQ ID NOs:328-339, SEQ ID NO:341, SEQ ID NO:343, SEQ ID NO:345, SEQ ID NO:347, SEQ ID NO:349, SEQ ID NO:351, SEQ ID NO:353, SEQ ID NO:354, SEQ ID NOs:356-361, SEQ ID NO:363, SEQ ID NO:365, and SEQ ID NOs:367-371. A tissue of a plant produced from the plant cell has a difference in the level of a carotenoid as compared to the corresponding level in tissue of a control plant that does not comprise the exogenous nucleic acid.

In another aspect, the invention features a method of producing a plant tissue. The method can comprise growing a plant cell comprising an exogenous nucleic acid, the exogenous nucleic acid comprising a regulatory region operably linked to a nucleotide sequence encoding a polypeptide, in which the HMM bit score of the amino acid sequence of the polypeptide is greater than 50, the HMM based on the amino acid sequences depicted in one of FIGS. 1-9, A tissue of a plant produced from the plant cell has a difference in the level of a carotenoid as compared to the corresponding level in tissue of a control plant that does not comprise the exogenous nucleic acid.

The nucleotide sequence in such methods can encode a polypeptide in which the HMM bit score of the amino acid sequence of the polypeptide is greater than 50, the HMM based on the amino acid sequences depicted in FIG. 7. The nucleotide sequence can encode a polypeptide comprising an amino acid sequence corresponding to SEQ ID NO: 110.

In another aspect, the invention features a method of modulating the level of a carotenoid in a plant. The method can comprise introducing into a plant cell an exogenous nucleic acid comprising a regulatory region operably linked to a nucleotide sequence having 80 percent or greater sequence identity to a nucleotide sequence selected from the group consisting of SEQ ID NO: 94, SEQ ID NO: 103, SEQ ID NO: 107, SEQ ID NO: 109, SEQ ID NO: 127, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO.: 145, SEQ ID NO: 147, SEQ ID NO: 149, SEQ ID NO: 151, SEQ ID NO: 153, SEQ ID NO: 155, SEQ ID NO: 158, SEQ ID NO: 160, SEQ ID NO: 162, SEQ ID NO: 164, SEQ ID NO: 166, SEQ ID NO: 168, SEQ ID NO: 171, SEQ ID NO: 173, SEQ ID NO: 175, SEQ ID NO: 177, SEQ ID NO: 179, SEQ ID NO: 181, SEQ ID NO: 184, SEQ ID NO: 186, SEQ ID NO: 188, SEQ ID NO: 190, SEQ ID NO: 192, SEQ ID NO: 194, SEQ ID NO: 197, SEQ ID NO: 204, SEQ ID NO: 207, SEQ ID NO: 209, SEQ ID NO: 211, SEQ ID NO: 214, SEQ ID NO: 217, SEQ ID NO: 219, SEQ ID NO: 221, SEQ ID NO: 225, SEQ ID NO: 227, SEQ ID NO: 230, SEQ ID NO: 233, SEQ ID NO: 235, SEQ ID NO: 237, SEQ ID NO: 239, SEQ ID NO: 241, SEQ ID NO: 243, SEQ ID NO: 245; SEQ ID NO: 247, SEQ ID NO: 258, SEQ ID NO: 260, SEQ ID NO: 263, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 314, SEQ ID NO: 317, SEQ ID NO: 319, SEQ ID NO: 321, SEQ ID NO: 323, SEQ ID NO: 325, SEQ ID NO: 327, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 355, SEQ ID NO: 362, SEQ ID NO: 364, SEQ ID NO: 366, SEQ ID NO: 372, SEQ ID NO: 374, SEQ ID NO: 375, SEQ ID NO: 376, SEQ ID NO: 377, SEQ ID NO: 378, SEQ ID NO: 379. In another aspect, the invention features a method of producing a plant tissue. The method can comprise growing a plant cell comprising an exogenous nucleic acid, the exogenous nucleic acid comprising a regulatory region operably linked to a nucleotide sequence having 80 percent or greater sequence identity to a nucleotide sequence selected from the aforementioned group of sequences. In both such methods, a tissue of a plant produced from the plant cell has a difference in the level of a carotenoid as compared to the corresponding level in tissue of a control plant that does not comprise the nucleic acid. The nucleotide sequence can encode a polypeptide, the HMM bit score of the amino acid sequence of the polypeptide being greater than 50, the HMM based on the amino acid sequences depicted in FIG. 7. The nucleotide sequence can encode a polypeptide comprising the amino acid sequence set forth in SEQ ID NO: 110. The nucleotide sequence can have a sequence identity that is 85 percent or greater to one of the aforementioned group of sequences.

The nucleotide sequence in any of the above methods can encode a polypeptide comprising an amino acid sequence selected from the group consisting of SEQ ID NO: 80, SEQ ID NO: 89, SEQ ID NO: 91, SEQ ID NO: 95, SEQ ID NO: 98, SEQ ID NO: 104, SEQ ID NO: 144 and SEQ ID NO: 240.

The carotenoid in any of the above methods can be phytoene, ζ-carotene, lycopene, δ-carotene, α-carotene, lutein, gamma-carotene, cis-β-carotene, trans-β-carotene, zeaxanthin, antheraxanthin, astaxanthin, bixin, capsanthin, fucoxanthin, or violaxanthin. The difference in the level of a carotenoid in any of the above methods can be an increase in the level of a carotenoid, e.g., ζ-carotene, lutein, cis-β-carotene, trans-β-carotene, neoxanthin, or violaxanthin. The regulatory region in any of the above methods can be a promoter, e.g., a tissue-preferential, broadly expressing, or inducible promoter.

The plant cell in any of the above methods can be from a member of a dicot genus, for example, Lycopersicon, Lactuca, Glycine, Gossypium, or Brassica. Alternatively, the plant cell can be from a member of a monocot genus, for example, Triticum, Zea, Oryza, or Musa.

The tissue in any of the above methods can be a fruit, vegetative, tuber, or seed tissue.

In another aspect, the invention features a plant cell comprising an exogenous nucleic acid. The exogenous nucleic acid comprises a regulatory region operably linked to a nucleotide sequence encoding a polypeptide, in which the HMM bit score of the amino acid sequence of the polypeptide is greater than 50, the HMM based on the amino acid sequences depicted in one of FIGS. 1-9. A tissue of a plant produced from the plant cell has a difference in the level of a carotenoid as compared to the corresponding level in tissue of a control plant that does not comprise the exogenous nucleic acid.

In another aspect, the invention features a plant cell comprising an exogenous nucleic acid, the exogenous nucleic acid comprising a regulatory region operably linked to a nucleotide sequence having 80 percent or greater sequence identity to a nucleotide sequence selected from the group consisting of SEQ ID NO: 94, SEQ ID NO: 103, SEQ ID NO: 107, SEQ ID NO: 109, SEQ ID NO: 127, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 145, SEQ ID NO: 147, SEQ ID NO: 149, SEQ ID NO: 151, SEQ ID NO: 153, SEQ ID NO: 155, SEQ ID NO: 158, SEQ ID NO: 160, SEQ ID NO: 162, SEQ ID NO: 164, SEQ ID NO: 166, SEQ ID NO: 168, SEQ ID NO: 171, SEQ ID NO: 173, SEQ ID NO: 175, SEQ ID NO: 177, SEQ ID NO: 179, SEQ ID NO: 181, SEQ ID NO: 184, SEQ ID NO: 186, SEQ ID NO: 188, SEQ ID NO: 190, SEQ ID NO: 192, SEQ ID NO: 194, SEQ ID NO: 197, SEQ ID NO: 204, SEQ ID NO: 207, SEQ ID NO: 209, SEQ ID NO: 211, SEQ ID NO: 214, SEQ ID NO: 217, SEQ ID NO: 219, SEQ ID NO: 221, SEQ ID NO: 225, SEQ ID NO: 227, SEQ ID NO: 230, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 258, SEQ ID NO: 260, SEQ ID NO: 263, SEQ ID NO: 266, SEQ ID NO: 268; SEQ ID NO: 270, SEQ ID NO: 272, SEQ ID NO: 274, SEQ ID NO: 276, SEQ ID NO: 278, SEQ ID NO: 280, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 314, SEQ ID NO: 317, SEQ ID NO: 319, SEQ ID NO: 321, SEQ ID NO: 323, SEQ ID NO: 325, SEQ ID NO: 327, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 355, SEQ ID NO: 362, SEQ ID NO: 364, SEQ ID NO: 366, SEQ ID NO: 372, SEQ ID NO: 374, SEQ ID NO: 375, SEQ ID NO: 376, SEQ ID NO: 377, SEQ ID NO: 378, SEQ ID NO: 379. A tissue of a plant produced from the plant cell has a difference in the level of a carotenoid as compared to the corresponding level in tissue of a control plant that does not comprise the nucleic acid. The nucleotide sequence can have a sequence identity that is 85 percent or greater.

The invention also features a transgenic plant comprising an exogenous nucleic acid. The exogenous nucleic acid comprises a regulatory region operably linked to a nucleotide sequence encoding a polypeptide, in which the HMM bit score of the amino acid sequence of the polypeptide is greater than 50, the HMM based on the amino acid sequences depicted in one of FIGS. 1-9. A tissue of a plant produced from the plant cell has a difference in the level of a carotenoid as compared to the corresponding level in tissue of a control plant that does not comprise the exogenous nucleic acid. The nucleotide sequence can encode a polypeptide in which the HMM bit score of the amino acid sequence of the polypeptide is greater than 50, the HMM based on the amino acid sequences depicted in FIG. 7. The nucleotide sequence can encode a polypeptide comprising an amino acid sequence corresponding to SEQ ID NO: 110. The nucleotide sequence can encode a polypeptide comprising an amino acid sequence selected from the group consisting of SEQ ID NO: 80, SEQ ID NO: 89, SEQ ID NO: 91, SEQ ID NO: 95, SEQ ID NO: 98, SEQ ID NO: 104, SEQ ID NO: 144 and SEQ ID NO: 240.

The carotenoid in any of the above plants or plant cells can be phytoene, ζ-carotene, lycopene, δ-carotene, α-carotene, lutein, gamma-carotene, cis-β-carotene, trans-β-carotene, zeaxanthin, antheraxanthin, astaxanthin, bixin, capsanthin, fucoxanthin, or violaxanthin. The difference in the level of a carotenoid in any of the above plants or plant cells can be an increase in the level of a carotenoid, e.g., ζ-carotene, lutein, cis-β-carotene, trans-β-carotene, neoxanthin, or violaxanthin. The regulatory region in any of the above plants or plant cells can be a promoter, e.g., a tissue-preferential, broadly expressing, or inducible promoter. The plants or plant cells can be from a member of a dicot genus, for example, Lycopersicon, Lactuca, Glycine, Gossypium, or Brassica. Alternatively, the plants or plant cells can be from a member of a monocot genus, for example, Triticum, Zea, Oryza, or Musa. Progeny of any of the above plants can have a difference in the level of a carotenoid as compared to the level of a carotenoid in a corresponding control plant that does not comprise the exogenous nucleic acid. The invention also features seed, vegetative tissue, and fruit from any of the above transgenic plants. In another aspect, the invention features a food product or a feed product comprising seed or vegetative tissue from any of the above transgenic plants. The tissue in any of the above plants or plant cells can be a fruit, vegetative, tuber, or seed tissue.

In another aspect, the invention features an isolated nucleic acid molecule comprising a nucleotide sequence having 95% or greater sequence identity to the nucleotide sequence selected from the group consisting of SEQ ID NO:105 and SEQ ID NO:107. In another aspect, the invention features an isolated nucleic acid comprising a nucleotide sequence encoding a polypeptide having 80% or greater sequence identity to the amino acid sequence selected from the group consisting of SEQ ID NO:106 and SEQ ID NO:108.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an alignment of Ceres CLONE ID no. 34553 (SEQ ID NO:80) with homologous and/or orthologous amino acid sequences Ceres CLONE ID no. 473423 (SEQ ID NO:81), Ceres CLONE ID no. 614555 (SEQ ID NO:82), Ceres CLONE ID no. 1329993 (SEQ ID NO:83), Ceres CLONE ID no. 258825 (SEQ ID NO:84), GI no. 34903896 (SEQ ID NO:85), GI no. 57900348 (SEQ ID NO:86), and GI no. 50906641 (SEQ ID NO:87). FIG. 1 and the other alignment figures provided herein were generated using the program MUSCLE version 3.52 (Edgar, Nucleic Acids Res, 32(5):1792-97 (2004); World Wide Web at drive5.com/muscle).

FIG. 2 is an alignment of Ceres CLONE ID no. 968026 (SEQ ID NO:91) with homologous and/or orthologous amino acid sequences GI no. 28466913 (SEQ ID NO:92) and Ceres CLONE ID no. 596510 (SEQ ID NO:93).

FIG. 3 is an alignment of Ceres CLONE ID no. 13930 (SEQ ID NO:104) with homologous and/or orthologous amino acid sequences Ceres ANNOT ID no. 1475265 (SEQ ID NO:108), Ceres CLONE ID no. 1842178 (SEQ ID NO:128), GI no. 18413971 (SEQ ID NO:129), and Ceres CLONE ID no. 1044646 (SEQ ID NO:131).

FIG. 4 is an alignment of Ceres CLONE ID no. 34589 (SEQ ID NO:95) with homologous and/or orthologous amino acid sequences Ceres CLONE ID no. 975220 (SEQ ID NO:96), Ceres CLONE ID no. 1973945 (SEQ ID NO:205), Ceres ANNOT ID no. 1501772 (SEQ ID NO:210), GI no. 15221257 (SEQ ID NO:213), Ceres CLONE ID no. 1371146 (SEQ ID NO:215), Ceres CLONE ID no. 1020930 (SEQ ID NO:218), Ceres CLONE ID no. 325641 (SEQ ID NO:222), and GI no. 116310449 (SEQ ID NO:223).

FIG. 5 is an alignment of Ceres CLONE ID no. 21863 (SEQ ID NO:89) with homologous and/or orthologous amino acid sequences Truncated Version of Ceres CLONE ID no. 1918241 (SEQ ID NO:159), Truncated Version of Ceres ANNOT ID no. 1454043 (SEQ ID NO:161), Truncated Version of Ceres ANNOT ID no. 1464854 (SEQ ID NO:165), Truncated Version of Ceres CLONE ID no. 479514 (SEQ ID NO:172), Truncated Version of Ceres CLONE ID no. 987194 (SEQ ID NO:178), Truncated Version of Ceres CLONE ID no. 1780447 (SEQ ID NO:180), Truncated Version of Ceres CLONE ID no. 677852 (SEQ ID NO:182), and Truncated Version of Public GI no. 115452185 (SEQ ID NO:183).

FIG. 6 is an alignment of GI no. 18424254 (SEQ ID NO:144) with homologous and/or orthologous amino acid sequences Ceres CLONE ID no. 1918241 (SEQ ID NO:133), Ceres ANNOT ID no. 1464854 (SEQ ID NO:137), Ceres CLONE ID no. 479514 (SEQ ID NO:146), Ceres CLONE ID no. 987194 (SEQ ID NO:152), Ceres CLONE ID no. 1780447 (SEQ ID NO:154), Ceres CLONE ID no. 677852 (SEQ ID NO:156), and GI no. 115452185 (SEQ ID NO:157).

FIG. 7 is an alignment of Ceres CLONE ID no. 641355 (SEQ ID NO:110) with homologous and/or orthologous amino acid sequences Ceres CLONE ID no. 1926437 (SEQ ID NO:294), Ceres ANNOT ID no. 1463335 (SEQ ID NO:296), GI no. 15239863 (SEQ ID NO:306), Ceres CLONE ID no. 1080500 (SEQ ID NO:320), Ceres CLONE ID no. 641355 (SEQ ID NO:322), Ceres CLONE ID no. 555364 (SEQ ID NO:326), and GI no. 31432356 (SEQ ID NO:331).

FIG. 8 is an alignment of Ceres CLONE ID no. 316638 (SEQ ID NO:98) with homologous and/or orthologous amino acid sequences Truncated Version of Ceres CLONE ID no. 1929841 (SEQ ID NO:259), Truncated Version of Ceres ANNOT ID no. 1470444 (SEQ ID NO:261), Truncated Version of Public GI no. 15237901 (SEQ ID NO:262), Truncated Version of Ceres CLONE ID no. 707855 (SEQ ID NO:269), Truncated Version of Ceres CLONE ID no. 757222 (SEQ ID NO:271), Truncated Version of Ceres CLONE ID no. 1545342 (SEQ ID NO:273), Truncated Version of Ceres CLONE ID no. 1860083 (SEQ ID NO:281), and Truncated Version of Public GI no. 41052966 (SEQ ID NO:283).

FIG. 9 is an alignment of Ceres CLONE ID no. 1545342 (SEQ ID NO:240) with homologous and/or orthologous amino acid sequences Ceres CLONE ID no. 1929841 (SEQ ID NO:226), Ceres ANNOT ID no. 1470444 (SEQ ID NO:228), GI no. 15237901 (SEQ ID NO:229), Ceres CLONE ID no. 707855 (SEQ ID NO:236), Ceres CLONE ID no. 757222 (SEQ ID NO:238), Ceres CLONE ID no. 1860083 (SEQ ID NO:248), and GI no. 41052966 (SEQ ID NO:250).

### DETAILED DESCRIPTION

Carotenoids are a diverse class of metabolites derived from isoprenoid units. The conjugated chain common to all carotenoids consists of eight isoprenoid units joined end-to-end. All carotenoids may be formally derived from the canonical polyene carotenoid structure through hydrogenation, dehydrogenation, cyclization, oxidation or any combination of these processes. Carotenoids can also include compounds that arise from degradations of the carbon skeleton, provided that the two central methyl groups are maintained. Carotenoids are generally divided into two classes: the carotenes, which are unoxidized, and the xanthophylls, in which some of the double bonds have been oxidized. Useful carotenoids include molecules from both classes, for example, α-, β-, and ζ-carotenes and xanthophylls, e.g. lutein and zeaxanthin.

The invention features methods and materials related to modulating (e.g., increasing or decreasing) carotenoid levels in plants. The methods can include transforming a plant cell with a nucleic acid encoding a carotenoid-modulating polypeptide, wherein expression of the polypeptide results in a modulated level of one or more carotenoids. Plant cells produced using such methods can be grown to produce plants having an increased or decreased carotenoid content. Such plants may be used to produce, for example, foodstuffs and animal feed having an increased nutritional content, and/or modified appearance or color, which may benefit both food producers and consumers, or can be used as sources from which to extract one or more carotenoids.

Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include carotenoid-modulating polypeptides. carotenoid-modulating polypeptides can be effective to modulate carotenoid levels when expressed in a plant or plant cell. Modulation of the level of carotenoid can be either an increase or a decrease in the level of carotenoid relative to the corresponding level in a control plant.

A carotenoid-modulating polypeptide can be a transcription factor, such as a basic-leucine zipper (bZIP) transcription factor polypeptide. Transcription factors are a diverse class of proteins that regulate gene expression through specific DNA binding events. Transcription factors are involved in a variety of regulatory networks of genes in plants, including those genes responsible for the biosynthesis of metabolites. Transcription factors include a number of characteristic structural motifs that mediate interactions with nucleic acids. The bZIP transcription factors of eukaryotes are polypeptide that contain a basic region mediating sequence-specific DNA-binding, followed by a leucine zipper region, which is required for dimerization. Such polypeptides can serve as structural platforms for DNA binding and/or protein-protein interactions. SEQ ID NO:80 sets forth the amino acid sequence of an Arabidopsis clone, identified herein as Ceres CLONE ID no. 34553 (SEQ ID NO:79), that is predicted to encode a bZIP transcription factor containing a bZIP_{—}2 domain.

A carotenoid-modulating polypeptide can comprise the amino acid sequence set forth in SEQ ID NO:80. Alternatively, a carotenoid-modulating polypeptide can be a homolog, ortholog, or variant of the polypeptide having the amino acid sequence set forth in SEQ ID NO:80. For example, a carotenoid-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO:80.

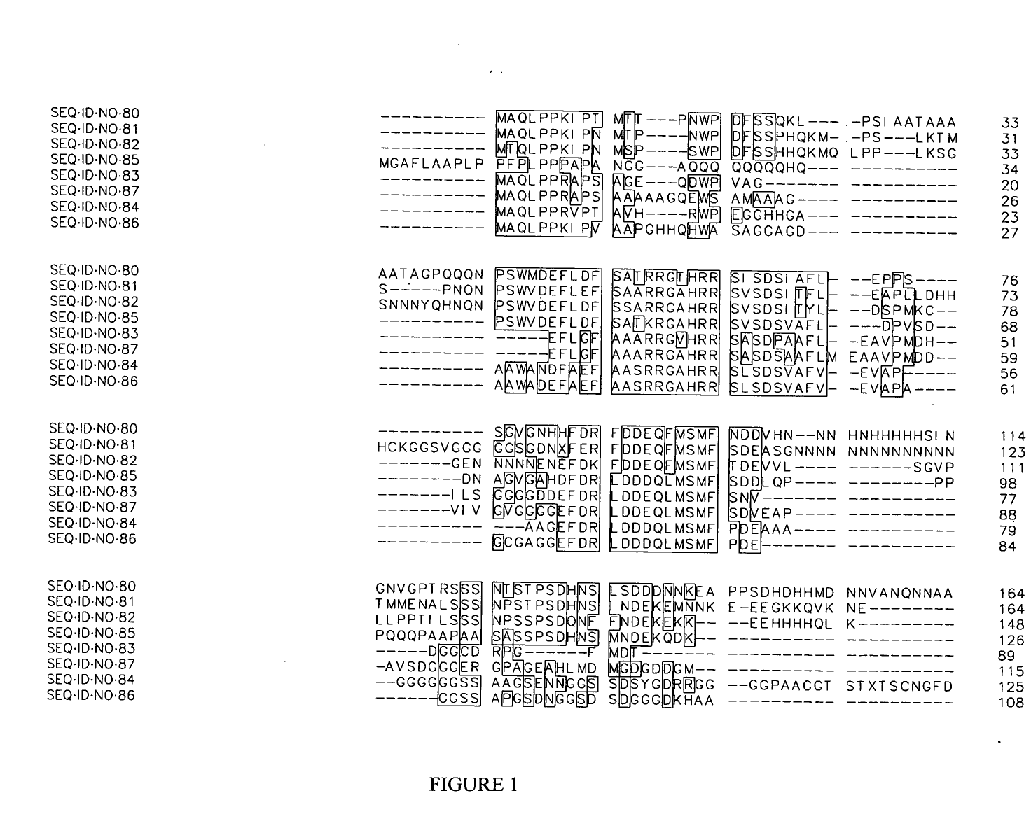

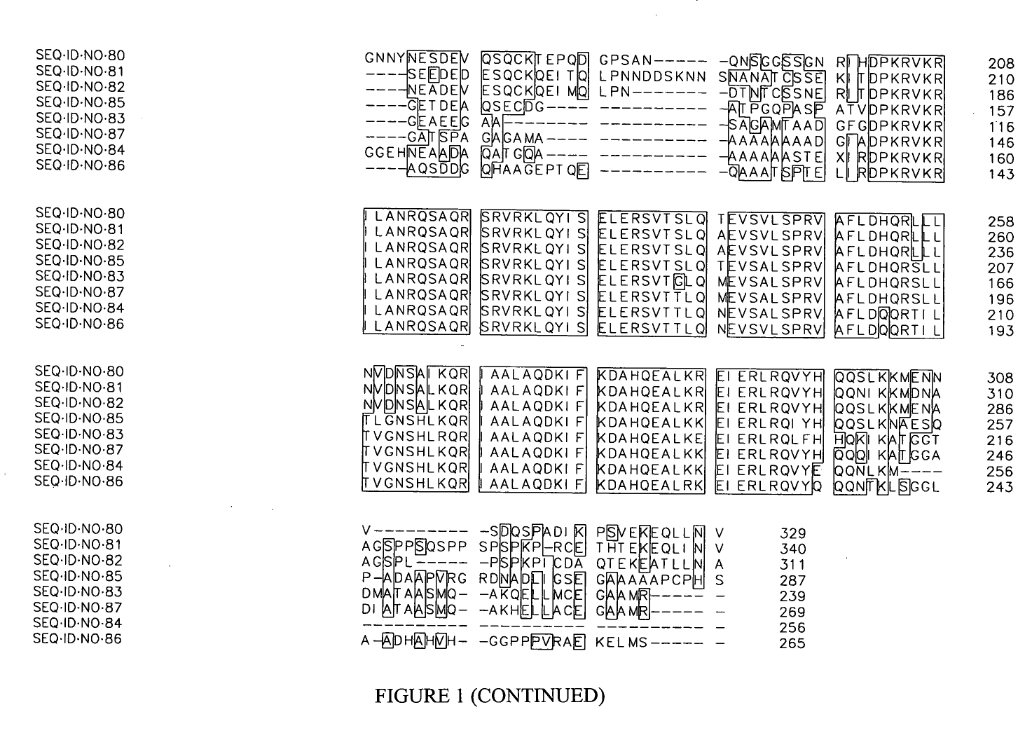

Amino acid sequences of homologs and/or orthologs of the polypeptide having the amino acid sequence set forth in SEQ ID NO:80 are provided in FIG. 1. The alignment in FIG. 1 provides the amino acid sequences of Ceres CLONE ID no. 34553 (SEQ ID NO:80), Ceres CLONE ID no. 473423 (SEQ ID NO:81), Ceres CLONE ID no. 614555 (SEQ ID NO:82), Ceres CLONE ID no. 1329993 (SEQ ID NO:83), Ceres CLONE ID no. 258825 (SEQ ID NO:84), GI no. 34903896 (SEQ ID NO:85), GI no. 57900348 (SEQ ID NO:86), and GI no. 50906641 (SEQ ID NO:87). Other homologs and/or orthologs include Ceres ANNOT ID no. 1534144 (SEQ ID NO:185), Ceres ANNOT ID no. 1441679 (SEQ ID NO:187), Ceres ANNOT ID no. 1480659 (SEQ ID NO:189), Ceres ANNOT ID no. 1479838 (SEQ ID NO:191), Ceres ANNOT ID no. 1533308 (SEQ ID NO:193), Ceres CLONE ID no. 463380 (SEQ ID NO:195), GI no. 113367174 (SEQ ID NO:196), Ceres CLONE ID no. 908192 (SEQ ID NO:198), GI no. 125538797 (SEQ ID NO:199), GI no. 115435234 (SEQ ID NO:200), GI no. 115440013 (SEQ ID NO:201), GI no. 125581476 (SEQ ID NO:202), and GI no. 115445299 (SEQ ID NO:203).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:81, SEQ ID NO:82, SEQ ID NO:83, SEQ ID NO:84, SEQ ID NO:85, SEQ ID NO:86, SEQ ID NO:87, SEQ ID NO:185, SEQ ID NO:187, SEQ ID NO:189, SEQ ID NO:191, SEQ ID NO:193, SEQ ID NO:195, SEQ ID NO:196, SEQ ID NO:198, SEQ ID NO:199, SEQ ID NO:200, SEQ ID NO:201, SEQ ID NO:202, or SEQ ID NO:203.

A carotenoid-modulating polypeptide can have a zf-C3HC4 domain characteristic of a C3HC4 type (RING finger) zinc-finger polypeptide. The RING finger is a specialized type of zinc-finger of 40 to 60 residues that binds two atoms of zinc and is reported to be involved in mediating polypeptide-polypeptide interactions. There are two different variants, the C3HC4-type and a C3H2C3-type, which are related despite the different cysteine/histidine pattern. The RING domain has been implicated in diverse biological processes. Ubiquitin-protein ligases (E3s), which determine the substrate specificity for ubiquitylation, have been classified into HECT and RING-finger families. Various RING fingers exhibit binding to E2 ubiquitin-conjugating enzymes. The ubiquitin-proteosome pathway is a key metabolic route for protein degradation in plant and animal cells. As proteins become destined for degradation, they are tagged by the covalent addition of one or more molecules of ubiquitin, a small protein that acts as a molecular marker that helps to direct the condemned protein to the proteosome, an organelle where proteolysis takes place. Ubiquitin protein ligases are a multigene family of enzymes responsible for the attachment of ubiquitin to lysine residues on the target proteins. Ceres CLONE ID no. 21863 (SEQ ID NO:89) is predicted to encode a polypeptide having a zf-C3HC4 domain. A carotenoid-modulating polypeptide can comprise the amino acid sequence set forth in SEQ ID NO:89. Alternatively, a carotenoid-modulating polypeptide can be a homolog, ortholog, or variant of the polypeptide having the amino acid sequence set forth in SEQ ID NO:89. For example, a carotenoid-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO:89.

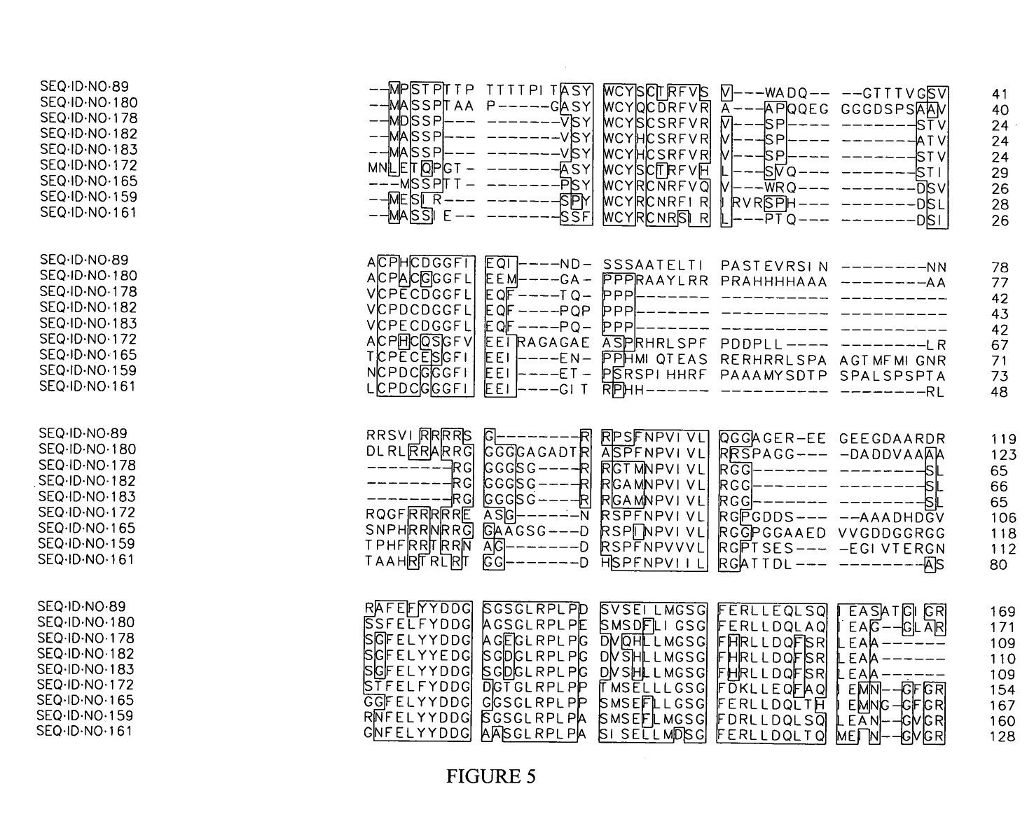

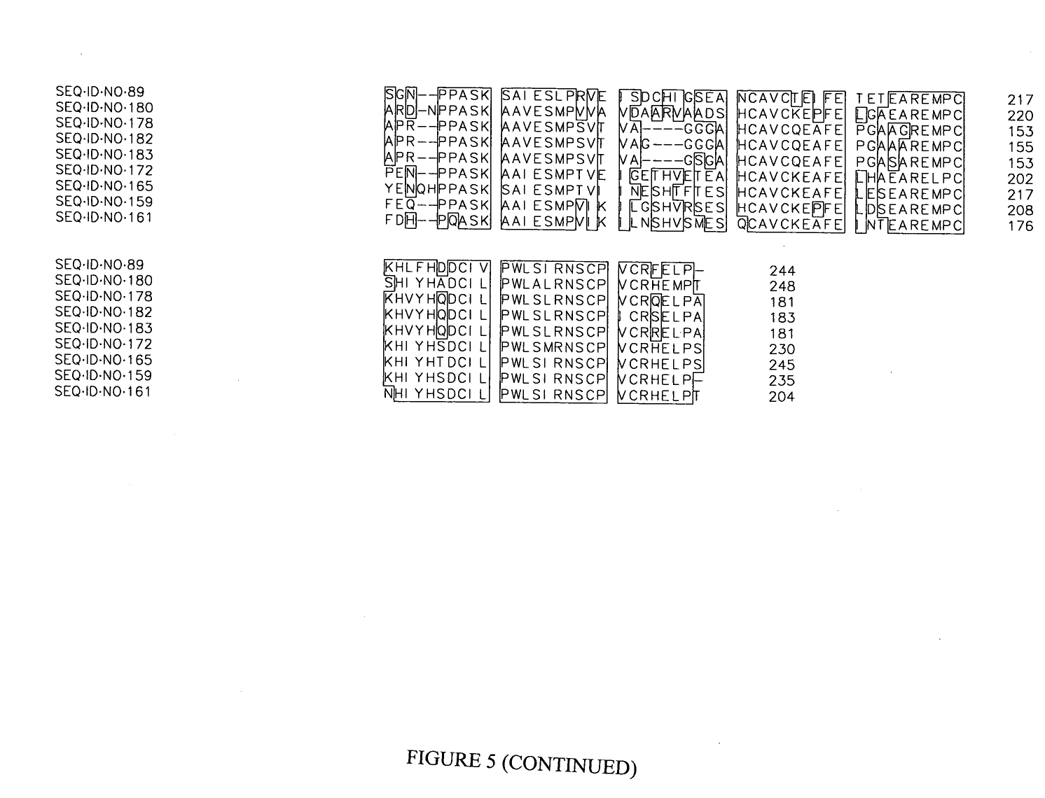

Amino acid sequences of homologs and/or orthologs of the polypeptide having the amino acid sequence set forth in SEQ ID NO:89 are provided in FIG. 5. The alignment in FIG. 5 provides the amino acid sequences of Ceres CLONE ID no. 21863 (SEQ ID NO:89), Truncated Version of Ceres CLONE ID no. 1918241 (SEQ ID NO:159), Truncated Version of Ceres ANNOT ID no. 1454043 (SEQ ID NO:161), Truncated Version of Ceres ANNOT ID no. 1464854 (SEQ ID NO:165), Truncated Version of Ceres CLONE ID no. 479514 (SEQ ID NO:172), Truncated Version of Ceres CLONE ID no. 987194 (SEQ ID NO:178), Truncated Version of Ceres CLONE ID no. 1780447 (SEQ ID NO:180), Truncated Version of Ceres CLONE ID no. 677852 (SEQ ID NO:182), and Truncated Version of Public GI no. 115452185 (SEQ ID NO:183). Other homologs and/or orthologs include Truncated Version of Ceres CLONE ID no. 1938564 (SEQ ID NO:163), Truncated Version of Ceres ANNOT ID no. 1511378 (SEQ ID NO:167), Truncated Version of Ceres ANNOT ID no. 1458137 (SEQ ID NO:169), Truncated Version of Public GI no. 18424254 (SEQ ID NO:170), Truncated Version of Ceres CLONE ID no. 1240790 (SEQ ID NO:174), and Truncated Version of Ceres CLONE ID no. 942216 (SEQ ID NO:176).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:159, SEQ ID NO:161, SEQ ID NO:163, SEQ ID NO:165, SEQ ID NO:167, SEQ ID NO:169, SEQ ID NO:170, SEQ ID NO:172, SEQ ID NO:174, SEQ ID NO:176, SEQ ID NO:178, SEQ ID NO:180, SEQ ID NO:182, or SEQ ID NO:183.

In some cases, a carotenoid-modulating polypeptide can contain a zf-C3HC4 domain described herein and a DUF1117 domain present in the carboxy-terminus of a number of plant polypeptides. SEQ ID NO:144 sets forth the amino acid sequence of a polypeptide having a zf-C3HC4 domain and a DUF1117 domain.

A carotenoid-modulating polypeptide can comprise the amino acid sequence set forth in SEQ ID NO:144. Alternatively, a carotenoid-modulating polypeptide can be a homolog, ortholog, or variant of the polypeptide having the amino acid sequence set forth in SEQ ID NO:144. For example, a carotenoid-modulating polypeptide can have an amino acid sequence with at least 40% sequence identity, e.g., 41%, 45%, 50%, 55%, 60%, 65%, 70%, 75%, 80%, 85%, 90%, 91%, 92%, 93%, 94%, 95%, 96%, 97%, 98%, or 99% sequence identity, to the amino acid sequence set forth in SEQ ID NO:144.

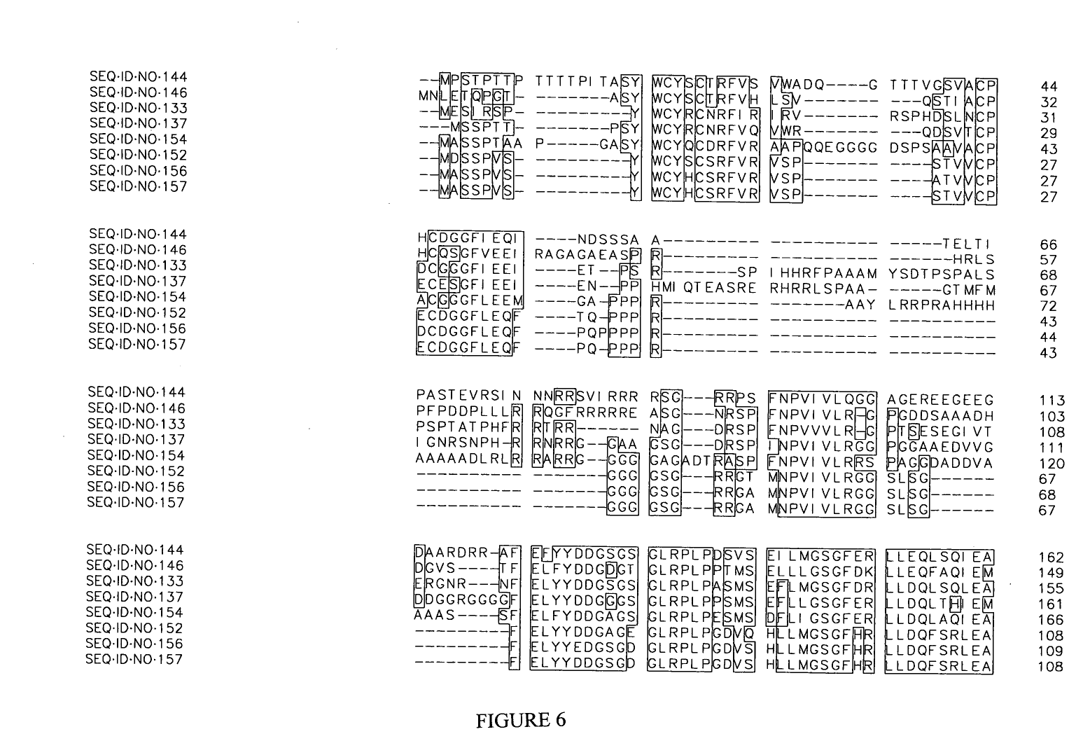

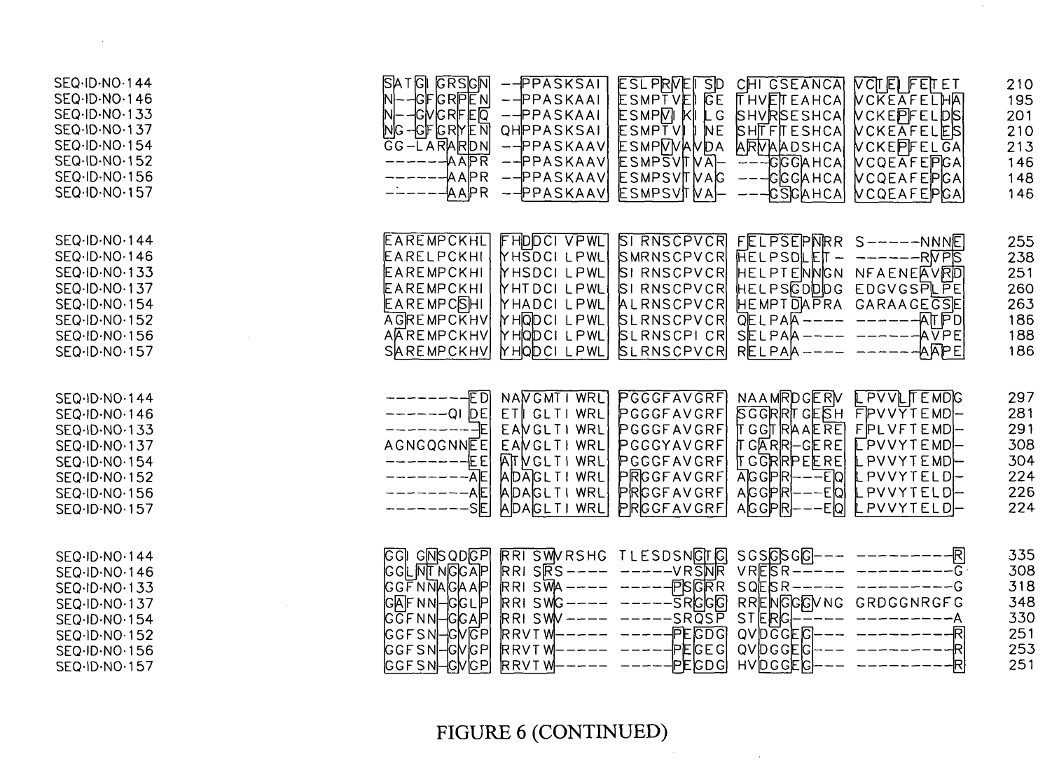

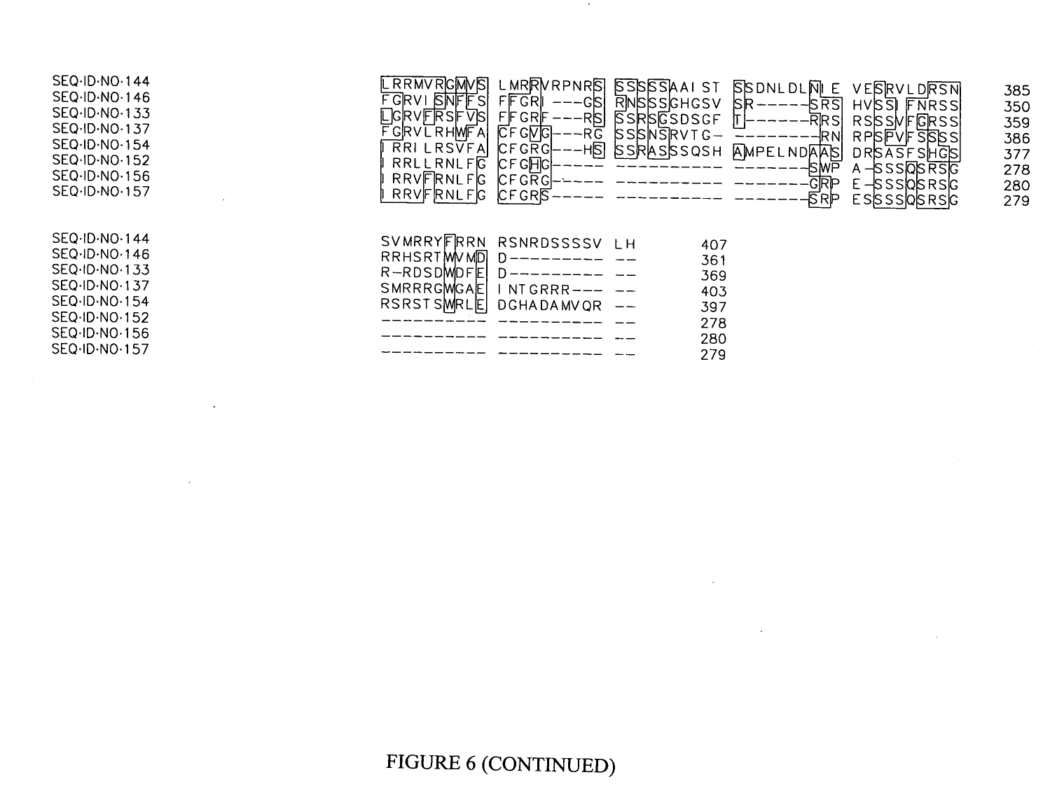

Amino acid sequences of homologs and/or orthologs of the polypeptide having the amino acid sequence set forth in SEQ ID NO:144 are provided in FIG. 6. The alignment in FIG. 6 provides the amino acid sequences of GI no. 18424254 (SEQ ID NO:144), Ceres CLONE ID no. 1918241 (SEQ ID NO:133), Ceres ANNOT ID no. 1464854 (SEQ ID NO:137), Ceres CLONE ID no. 479514 (SEQ ID NO:146), Ceres CLONE ID no. 987194 (SEQ ID NO:152), Ceres CLONE ID no. 1780447 (SEQ ID NO:154), Ceres CLONE ID no. 677852 (SEQ ID NO:156), and GI no. 115452185 (SEQ ID NO:157). Other homologs and/or orthologs include Ceres CLONE ID no. 1938564 (SEQ ID NO:135), Ceres ANNOT ID no. 1511378 (SEQ ID NO:139), Ceres ANNOT ID no. 1458137 (SEQ ID NO:141), Ceres ANNOT ID no. 1454043 (SEQ ID NO:143), Ceres CLONE ID no. 1240790 (SEQ ID NO:148), and Ceres CLONE ID no. 942216 (SEQ ID NO:150).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:133, SEQ ID NO:135, SEQ ID NO:137, SEQ ID NO:139, SEQ ID NO:141, SEQ ID NO:143, SEQ ID NO:146, SEQ ID NO:148, SEQ ID NO:150, SEQ ID NO:152, SEQ ID NO:154, SEQ ID NO:156, or SEQ ID NO:157.

A carotenoid-modulating polypeptide can be a response regulator protein. Response regulator proteins are a family of polypeptides that function as phosphoaccepting receivers in the histidine-aspartate phosphorelay signal transduction system. Response regulators play critical roles in cytokinin signaling circuitry and as such, are involved in a wide range of cellular processes including circadian rhythms and light-signal responses. SEQ ID NO:91 sets forth the amino acid sequence of a Brassica napus clone, identified herein as Ceres CLONE ID no. 968026 (SEQ ID NO:90), that is predicted to encode an amino acid response regulator 6 protein.

A carotenoid-modulating polypeptide can comprise the amino acid sequence set forth in SEQ ID NO:91. Alternatively, a carotenoid-modulating polypeptide can be a homolog, ortholog, or variant of the polypeptide having the amino acid sequence set forth in SEQ ID NO:91. For example, a carotenoid-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO:91.

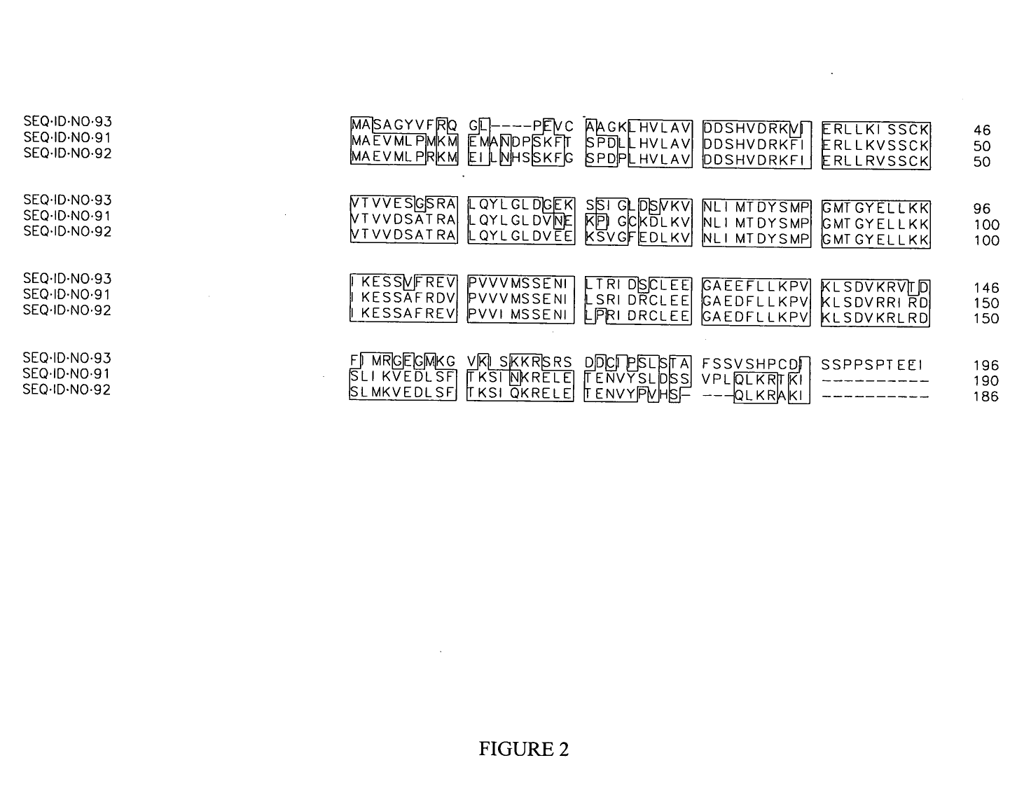

Amino acid sequences of homologs and/or orthologs of the polypeptide having the amino acid sequence set forth in SEQ ID NO:91 are provided in FIG. 2. The alignment in FIG. 2 provides the amino acid sequences of Ceres CLONE ID no. 968026 (SEQ ID NO:91), GI no. 28466913 (SEQ ID NO:92), and Ceres CLONE ID no. 596510 (SEQ ID NO:93). Other homologs and/or orthologs include Ceres CLONE ID no. 1832286 (SEQ ID NO:341), Ceres CLONE ID no. 1932920 (SEQ ID NO:343), Ceres ANNOT ID no. 1473516 (SEQ ID NO:345), Ceres ANNOT ID no. 1526929 (SEQ ID NO:347), Ceres ANNOT ID no. 1474764 (SEQ ID NO:349), Ceres ANNOT ID no. 1443270 (SEQ ID NO:351), Ceres ANNOT ID no. 1496190 (SEQ ID NO:353), GI no. 15242000 (SEQ ID NO:354), Ceres CLONE ID no. 34579 (SEQ ID NO:356), GI no. 15228338 (SEQ ID NO:357), GI no. 3953599 (SEQ ID NO:358), GI no. 3323581 (SEQ ID NO:359), GI no. 3953605 (SEQ ID NO:360), GI no. 15230202 (SEQ ID NO:361), Ceres CLONE ID no. 1240183 (SEQ ID NO:363), Ceres CLONE ID no. 775387 (SEQ ID NO:365), Ceres CLONE ID no. 916238 (SEQ ID NO:367), GI no. 12060388 (SEQ ID NO:368), GI no. 90265238 (SEQ ID NO:369), GI no. 115484121 (SEQ ID NO:370), and GI no. 87116390 (SEQ ID NO:371).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:92, SEQ ID NO:93, SEQ ID NO:341, SEQ ID NO:343, SEQ ID NO:345, SEQ ID NO:347, SEQ ID NO:349, SEQ ID NO:351, SEQ ID NO:353, SEQ ID NO:354, SEQ ID NO:356, SEQ ID NO:357, SEQ ID NO:358, SEQ ID NO:359, SEQ ID NO:360, SEQ ID NO:361, SEQ ID NO:363, SEQ ID NO:365, SEQ ID NO:367, SEQ ID NO:368, SEQ ID NO:369, SEQ ID NO:370, and SEQ ID NO:371.

A carotenoid-modulating polypeptide can contain an AP2 domain characteristic of polypeptides belonging to the AP2/EREBP family of plant transcription factor polypeptides. AP2 (APETALA2) and EREBPs (ethylene-responsive element binding proteins) are prototypic members of a family of transcription factors unique to plants, whose distinguishing characteristic is that they contain the so-called AP2 DNA binding domain. AP2/EREBP genes form a large multigene family encoding polypeptides that are key regulators of several developmental processes, such as floral organ identity determination and control of leaf epidermal cell identity, and forming part of the mechanisms used by plants to respond to various types of biotic and environmental stress. For example, AP2 DNA-binding domains are common to many proteins involved in the regulation of ethylene, jasmonate and abscisic acid signaling pathways and responses to environmental stress such as cold, drought and high salt conditions. SEQ ID NO:110 sets forth the amino acid sequence of an Arabidopsis thaliana clone, identified herein as Ceres CLONE ID no. 641355, that is predicted to encode a polypeptide having an AP2 domain.

A carotenoid-modulating polypeptide can be an Arabidopsis polypeptide having the amino acid sequence set forth in SEQ ID NO:110. Alternatively, a carotenoid-modulating polypeptide can be a homolog, ortholog, or variant of the polypeptide having the amino acid sequence set forth in SEQ ID NO:110. For example, a carotenoid-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO:110.

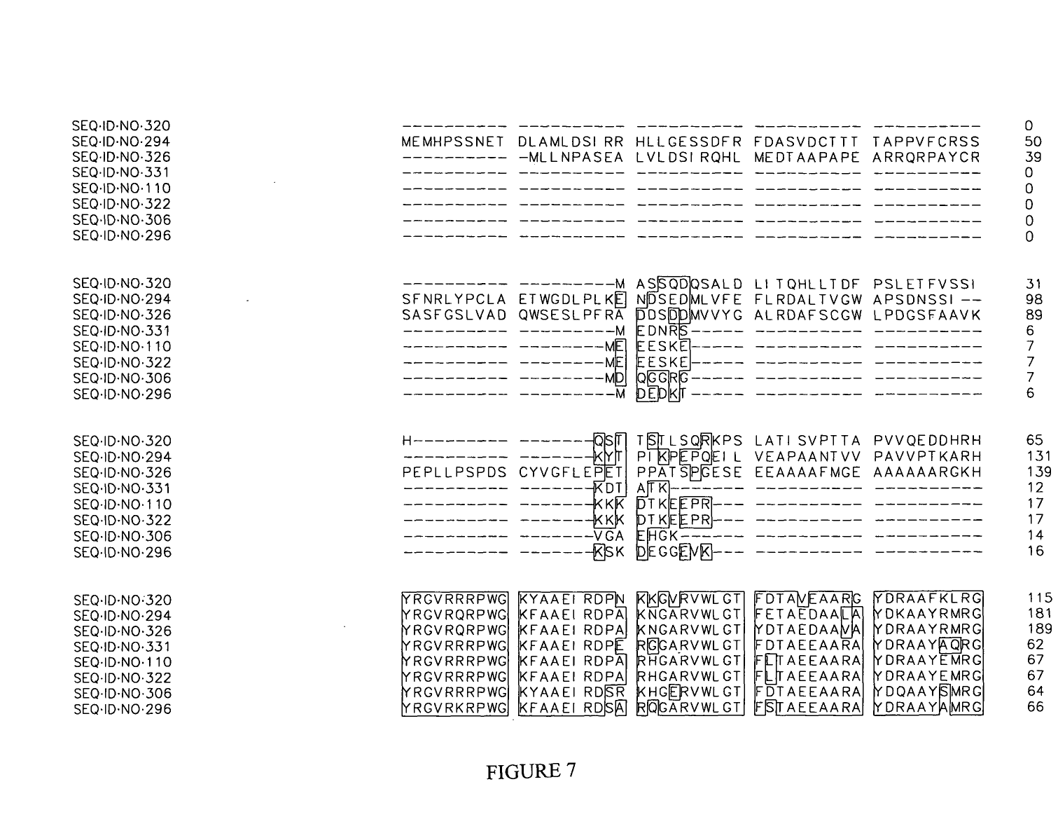

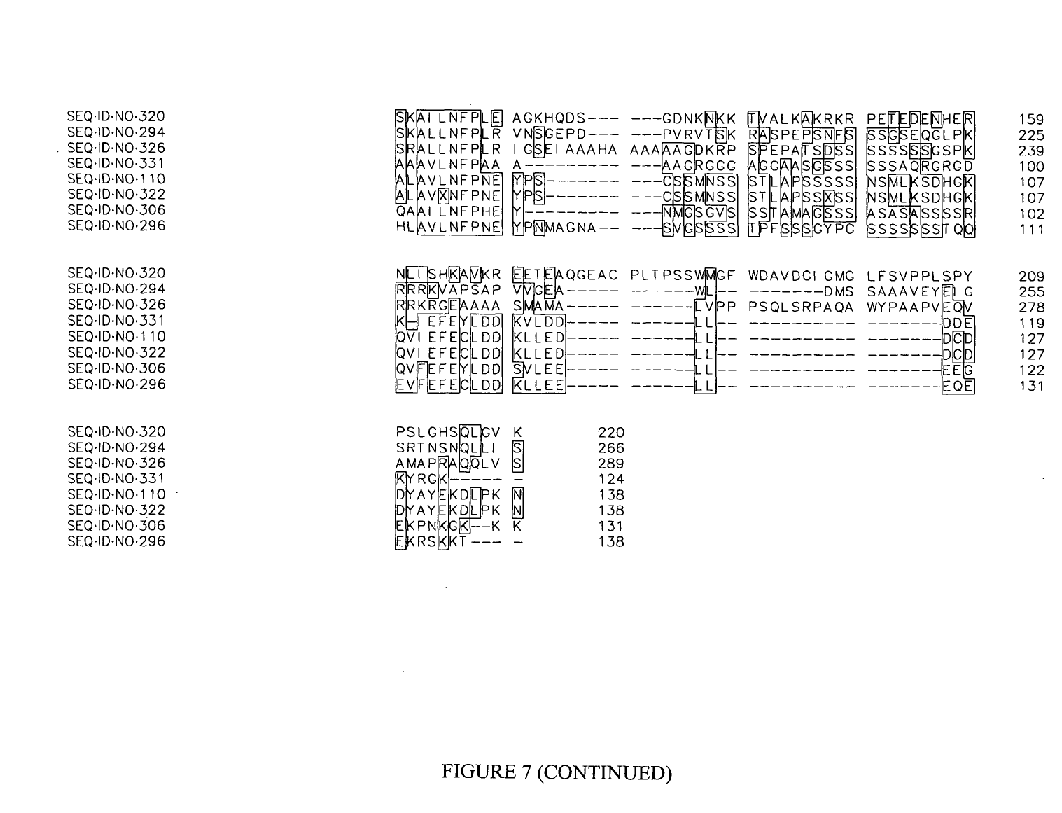

Amino acid sequences of homologs and/or orthologs of the polypeptide having the amino acid sequence set forth in SEQ ID NO:110 are provided in FIG. 7. The alignment in FIG. 7 provides the amino acid sequences of Ceres CLONE ID no. 641355 (SEQ ID NO:110), Ceres CLONE ID no. 1926437 (SEQ ID NO:294), Ceres ANNOT ID no. 1463335 (SEQ ID NO:296), GI no. 15239863 (SEQ ID NO:306), Ceres CLONE ID no. 1080500 (SEQ ID NO:320), Ceres CLONE ID no. 641355 (SEQ ID NO:322), Ceres CLONE ID no. 555364 (SEQ ID NO:326), and GI no. 31432356 (SEQ ID NO:331). Other homologs and/or orthologs include Ceres CLONE ID no. 1849534 (SEQ ID NO:292), Ceres ANNOT ID no. 1463334 (SEQ ID NO:298), Ceres ANNOT ID no. 1442765 (SEQ ID NO:300), Ceres ANNOT ID no. 1442760 (SEQ ID NO:302), Ceres ANNOT ID no. 1446840 (SEQ ID NO:304), GI no. 42565130 (SEQ ID NO:305), GI no. 116831575 (SEQ ID NO:307), GI no. 48479320 (SEQ ID NO:308), GI no. 145338854 (SEQ ID NO:309), GI no. 48479286 (SEQ ID NO:310), GI no. 21264420 (SEQ ID NO:311), GI no. 25350257 (SEQ ID NO:312), GI no. 25350258 (SEQ ID NO:313), Ceres CLONE ID no. 6042 (SEQ ID NO:315), GI no. 18414897 (SEQ ID NO:316), Ceres CLONE ID no. 965028 (SEQ ID NO:318), Ceres CLONE ID no. 907605 (SEQ ID NO:324), Ceres CLONE ID no. 569593 (SEQ ID NO:328), GI no. 125547473 (SEQ ID NO:329), GI no. 125562586 (SEQ ID NO:330), GI no. 125574952 (SEQ ID NO:332), GI no. 115450749 (SEQ ID NO:333), GI no. 125584936 (SEQ ID NO:334), GI no. 115457454 (SEQ ID NO:335), GI no. 52076099 (SEQ ID NO:336), GI no. 28071302 (SEQ ID NO:337), GI no. 115439973 (SEQ ID NO:338), and GI no. 57899163 (SEQ ID NO:339).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:292, SEQ ID NO:294, SEQ ID NO:296, SEQ ID NO:298, SEQ ID NO:300, SEQ ID NO:302, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:315, SEQ ID NO:316, SEQ ID NO:318, SEQ ID NO:320, SEQ ID NO:322, SEQ ID NO:324, SEQ ID NO:326, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, or SEQ ID NO:339.

A carotenoid-modulating polypeptide can be a tetratricopeptide repeat domain-containing protein. Tetratricopeptide repeats (TPR) are degenerate motifs that include a 34 amino acid long pair of anti-parallel α-helices linked by a short loop region. TPRs are often further arranged into large super-helical arrays of between 3 and 20 repeats that provide a contiguous surface involved in mediating protein-protein interactions. TPR domain proteins are involved in the regulation of a range of cellular and developmental processes, including protein translocation, asymmetric cell divisions and hormone signaling. Ceres Clone 13930 (SEQ ID NO: 104) is predicted to encode a TPR domain protein.

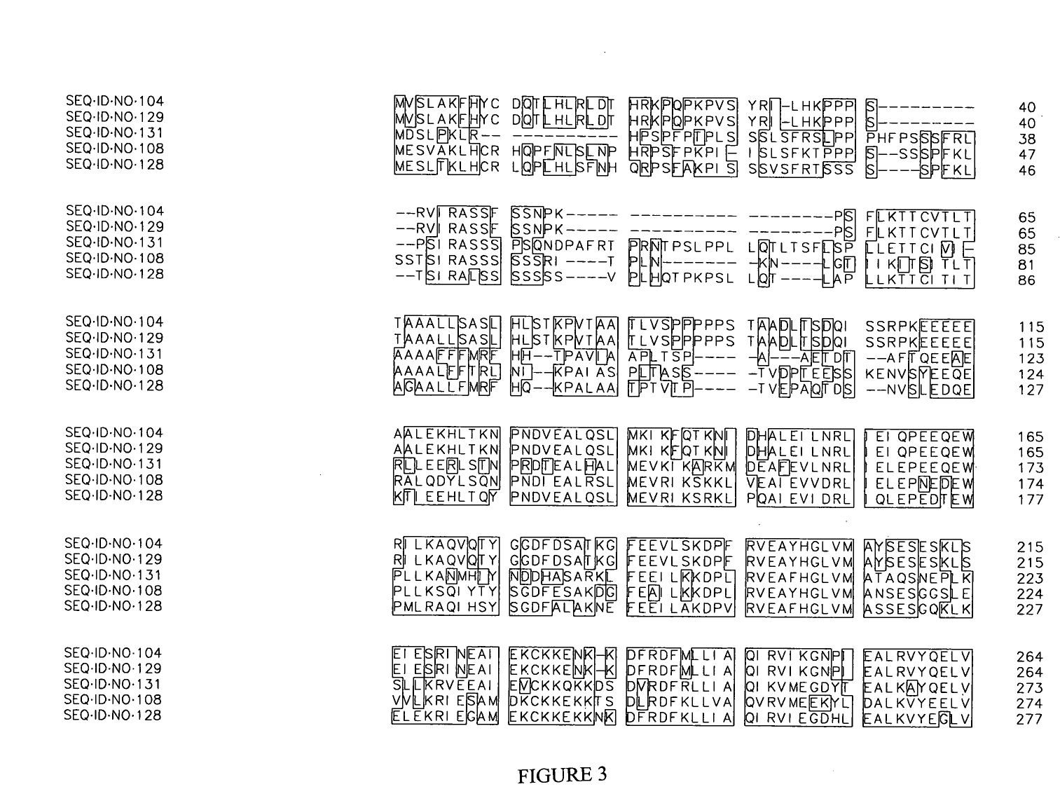

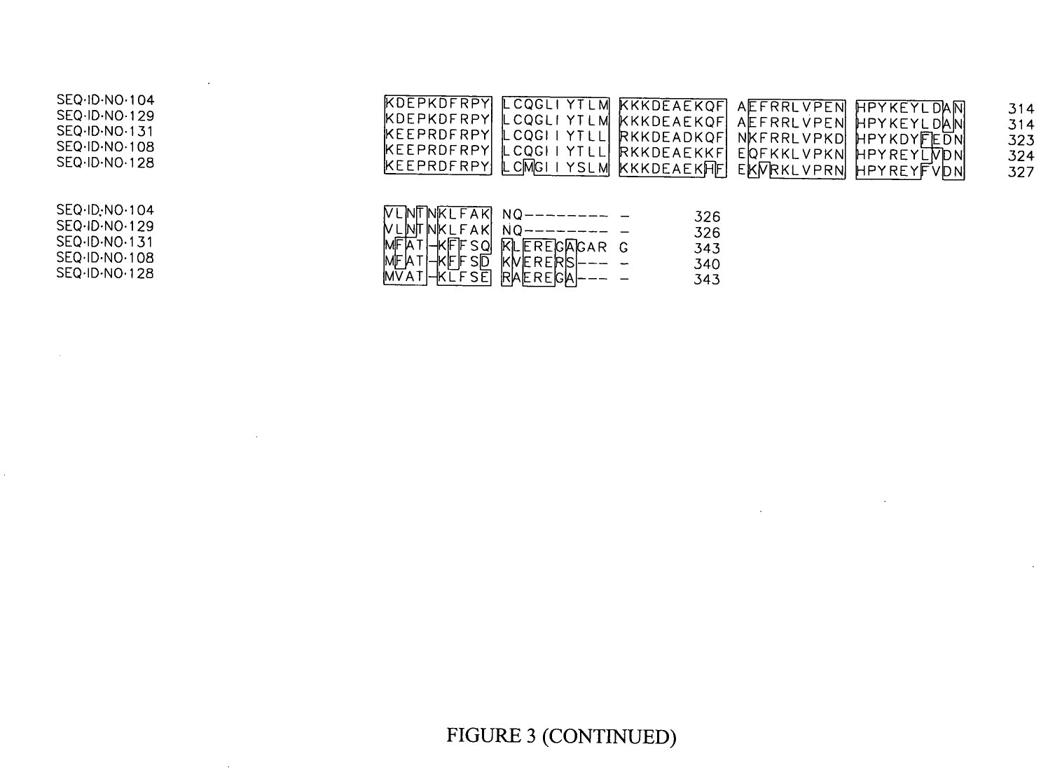

A carotenoid-modulating polypeptide can be an Arabidopsis polypeptide having the amino acid sequence set forth in SEQ ID NO: 104. Alternatively, a carotenoid-modulating polypeptide can be a homolog, ortholog, or variant of the polypeptide having the amino acid sequence set forth in SEQ ID NO: 104. For example, a carotenoid-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO: 104. Amino acid sequences of homologs and/or orthologs of the polypeptide having the amino acid sequence set forth in SEQ ID NO: 104 are provided in FIG. 3. The alignment in FIG. 3 provides the amino acid sequences of Ceres Clone 13930 (SEQ ID NO: 104), Public GI no. 18413971 (SEQ ID NO: 129), Ceres CLONE ID no. 1044646 (SEQ ID NO: 131), Ceres ANNOT ID no. 1475265 (SEQ ID NO: 108) and Ceres CLONE ID no. 1842178 (SEQ ID NO: 128). Other homologs and/or orthologs include Ceres ANNOT ID no. 1455046 (SEQ ID 106). In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO: 129, SEQ ID NO: 106, SEQ ID NO: 108, SEQ ID NO: 131, or SEQ ID NO: 128.

A carotenoid-modulating polypeptide can be a Myb-like DNA binding domain-containing protein. Myb-like DNA-binding domains are characteristic of a multi-gene family of DNA-binding proteins that specifically recognize the sequence YAAC(G/T)G. ‘Classical’ MYB factors, which are related to the protooncogene, c-MYB, are involved in the control of the cell cycle, while R2R3-type MYB genes control many aspects of plant secondary metabolism, as well as the identity and fate of plant cells. Ceres Clone 34589 (SEQ ID NO: 95) is predicted to encode a Myb-like DNA binding domain-containing polypeptide.

A carotenoid-modulating polypeptide can be an Arabidopsis polypeptide having the amino acid sequence set forth in SEQ ID NO: 95. Alternatively, a carotenoid-modulating polypeptide can be a homolog, ortholog, or variant of the polypeptide having the amino acid sequence set forth in SEQ ID NO: 95. For example, a carotenoid-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO: 95.

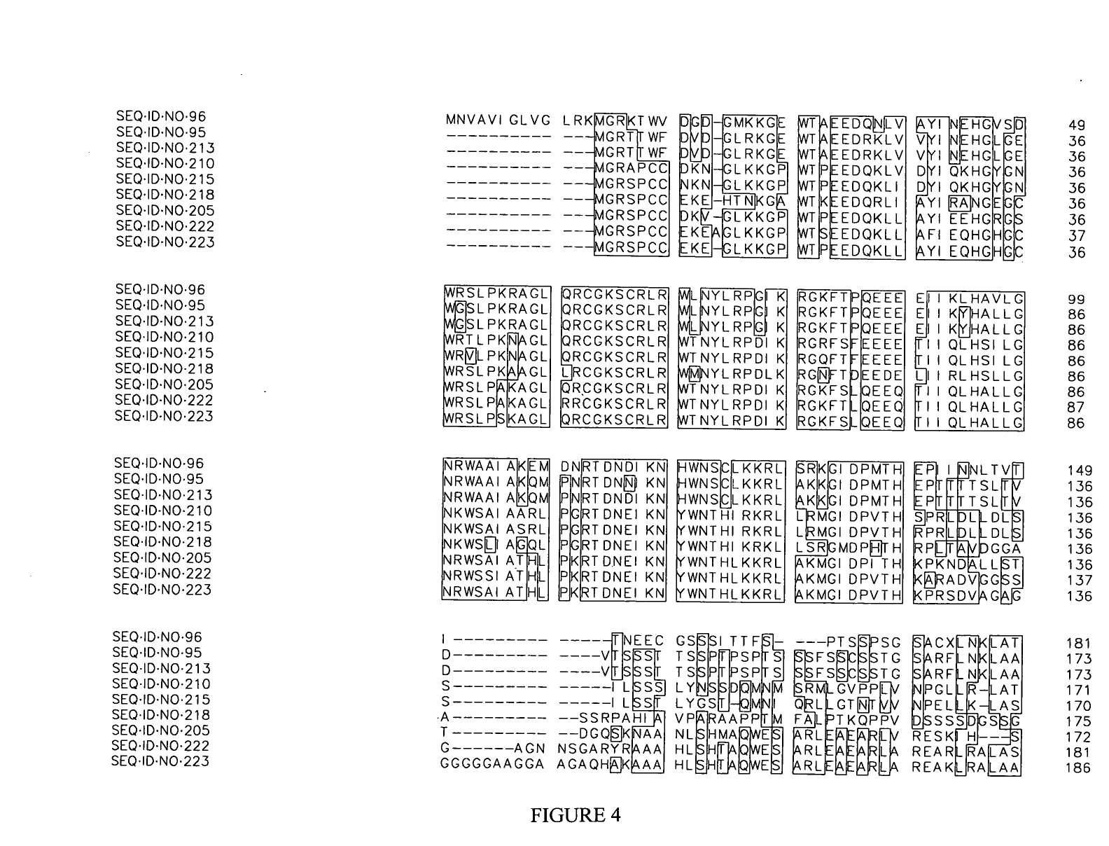

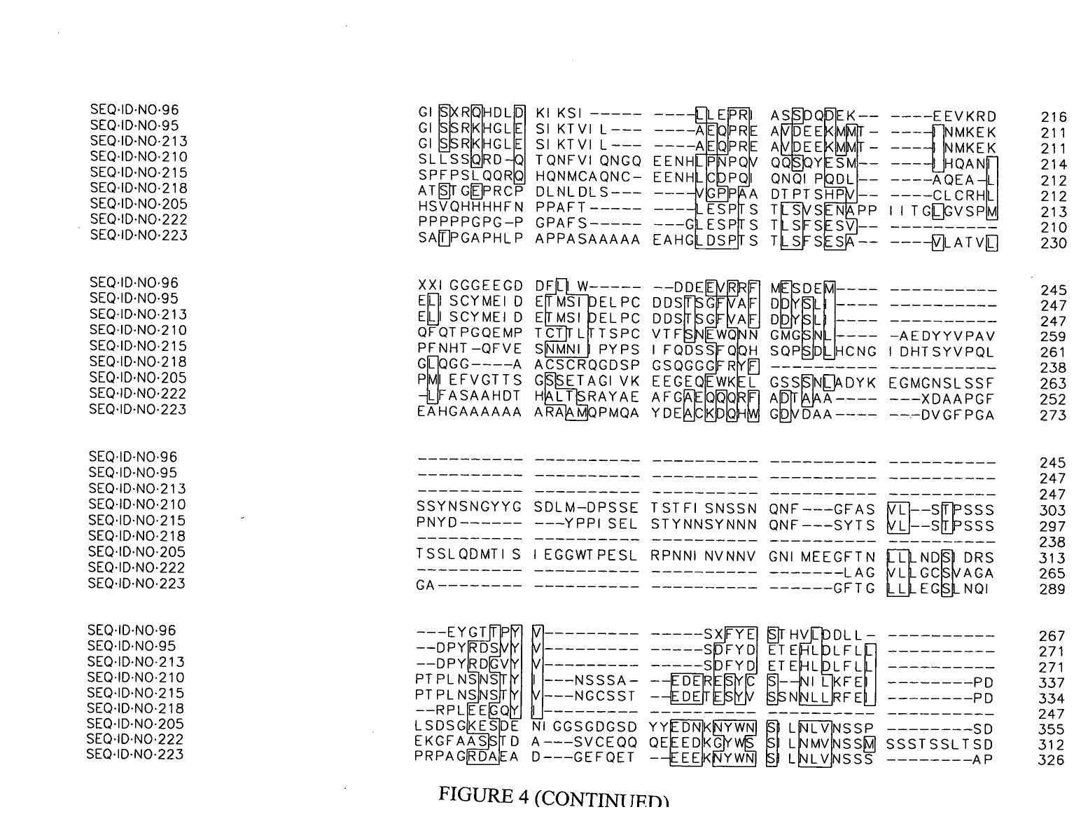

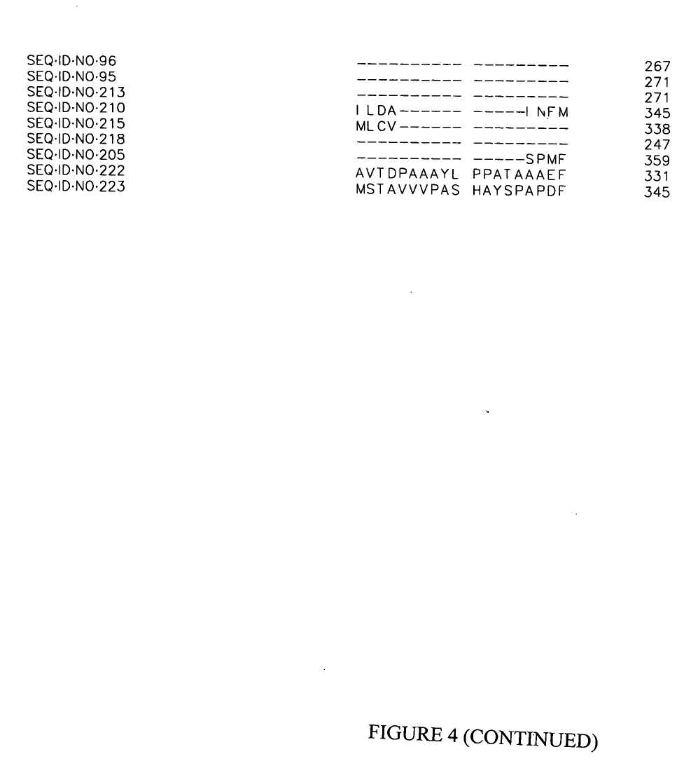

Amino acid sequences of homologs and/or orthologs of the polypeptide having the amino acid sequence set forth in SEQ ID NO: 95 are provided in FIG. 4. The alignment in FIG. 4 provides the amino acid sequences of Ceres Clone 34589 (SEQ ID NO: 95), Public GI no. 15221257 (SEQ ID NO: 213), Ceres ANNOT ID no. 1501772 (SEQ ID NO: 210), Ceres CLONE ID no. 1371146 (SEQ ID NO: 215), Ceres CLONE ID no. 1020930 (SEQ ID NO: 218), Ceres CLONE ID no. 325641 (SEQ ID NO: 222), Ceres Clone 975220 (SEQ ID NO: 96), Ceres CLONE ID no. 1973945 (SEQ ID NO: 205, and Public GI no. 116310449 (SEQ ID NO: 223). Other homologs and/or orthologs include Public GI no. 13346188 (SEQ ID NO: 206), Ceres ANNOT ID no. 1448769 (SEQ ID NO: 208), Ceres ANNOT ID no. 1465830 (SEQ ID NO: 212), Public GI no. 110931782 (SEQ ID NO: 216), Ceres CLONE ID no. 764797 (SEQ ID NO: 220) and Public GI no. 115458786 (SEQ ID NO: 224).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO: 96, SEQ ID NO: 213, SEQ ID NO: 210, SEQ ID NO: 215, SEQ ID NO: 218, SEQ ID NO: 205, SEQ ID NO: 222, SEQ ID NO: 206, SEQ ID NO: 208, SEQ ID NO: 212, SEQ ID NO: 216, SEQ ID NO: 220, SEQ ID NO: 223 and SEQ ID NO: 224.

A carotenoid-modulating polypeptide can be an ADP ribosylation factor. ADP ribosylation factors (ARFs) are members of the Ras superfamily of GTP binding proteins. ARFs are highly conserved, ubiquitously expressed in eukaryotic cells and appear to be involved in vesicular protein transport. Vesicle trafficking in plants is important in the delivery of proteins to intra- and extra-cellular compartments, e.g., the delivery of cellulose synthase to the plasma membrane and non-cellulosic polysaccharides to the cell wall. Ceres Clone 316638 (SEQ ID NO: 98) is predicted to encode an ADP ribosylation factor polypeptide.

A carotenoid-modulating polypeptide can be an Arabidopsis polypeptide having the amino acid sequence set forth in SEQ ID NO: 98. Alternatively, a carotenoid-modulating polypeptide can be a homolog, ortholog, or variant of the polypeptide having the amino acid sequence set forth in SEQ ID NO: 98. For example, a carotenoid-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO: 98.

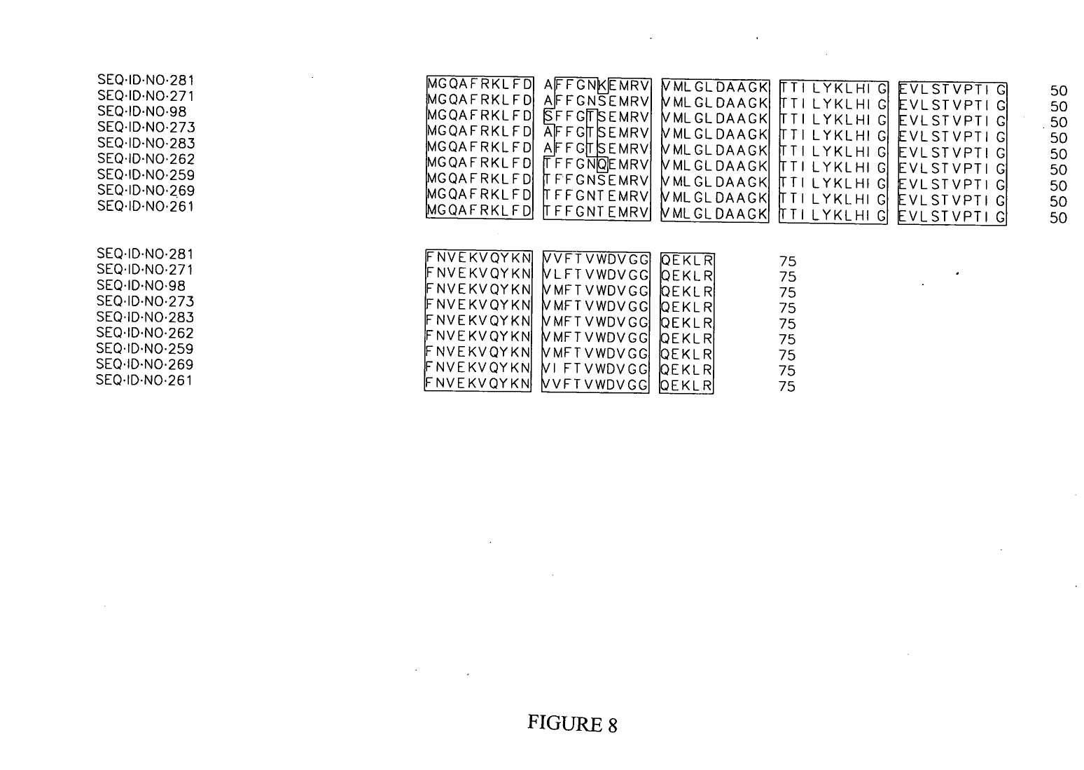

Amino acid sequences of homologs and/or orthologs of the polypeptide having the amino acid sequence set forth in SEQ ID NO: 98 are provided in FIG. 8. The alignment in FIG. 8 provides the amino acid sequences of Ceres Clone 316638 (SEQ ID NO: 98), Ceres CLONE ID no. 316638 (SEQ ID NO:98) with homologous and/or orthologous amino acid sequences Truncated Version of Ceres CLONE ID no. 1929841 (SEQ ID NO:259), Truncated Version of Ceres ANNOT ID no. 1470444 (SEQ ID NO:261), Truncated Version of Public GI no. 15237901 (SEQ ID NO:262), Truncated Version of Ceres CLONE ID no. 707855 (SEQ ID NO:269), Truncated Version of Ceres CLONE ID no. 757222 (SEQ ID NO:271), Truncated Version of Ceres CLONE ID no. 1545342 (SEQ ID NO:273), Truncated Version of Ceres CLONE ID no. 1860083 (SEQ ID NO:281), and Truncated Version of Public GI no. 41052966 (SEQ ID NO:283).

Other homologs and/or orthologs include Ceres CLONE ID no. 973476 (SEQ ID NO: 100), Ceres Clone 911175 (SEQ ID NO: 101), Truncated Version Of Ceres CLONE ID no. 26844 (SEQ ID NO: 264), Truncated Version Of Public GI no. 15228464 (SEQ ID NO: 265), Truncated Version Of Ceres CLONE ID no. 124385 (SEQ ID NO: 267), Truncated Version Of Ceres CLONE ID no. 275632 (SEQ ID NO: 275), Truncated Version Of Ceres CLONE ID no. 306269 (SEQ ID NO: 277), Truncated Version Of Ceres CLONE ID no. 295292 (SEQ ID NO: 279), Truncated Version Of Public GI no. 125537931 (SEQ ID NO: 282), Truncated Version Of Public GI no. 115448099 (SEQ ID NO: 284), Truncated Version Of Public GI no. 115483682 (SEQ ID NO: 285), Truncated Version Of Public GI no. 78709066 (SEQ ID NO: 286), Truncated Version Of Public GI no. 110289664 (SEQ ID NO: 287), Truncated Version Of Public GI no. 115443985 (SEQ ID NO: 288), Truncated Version Of Public GI no. 125580669 (SEQ ID NO: 289) and Truncated Version Of Public GI no. 7248402 (SEQ ID NO: 290).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO: 100, SEQ ID NO: 101, SEQ ID NO: 264, SEQ ID NO: 265, SEQ ID NO: 267, SEQ ID NO: 275, SEQ ID NO: 277, SEQ ID NO: 279, SEQ ID NO: 282, SEQ ID NO: 284, SEQ ID NO: 285, SEQ ID NO: 286, SEQ ID NO: 287, SEQ ID NO: 288, SEQ ID NO: 289 and SEQ ID NO: 290.

In some cases, a carotenoid-modulating polypeptide can be an ADP ribosylation factor having the amino acid sequence set forth in SEQ ID NO: 240. Alternatively, a carotenoid-modulating polypeptide can be a homolog, ortholog, or variant of the polypeptide having the amino acid sequence set forth in SEQ ID NO:240. For example, a carotenoid-modulating polypeptide can have an amino acid sequence with at least 40% sequence identity, e.g., 41%, 45%, 50%, 55%, 60%, 65%, 70%, 75%, 80%, 85%, 90%, 91%, 92%, 93%, 94%, 95%, 96%, 97%, 98%, or 99% sequence identity, to the amino acid sequence set forth in SEQ ID NO:240.

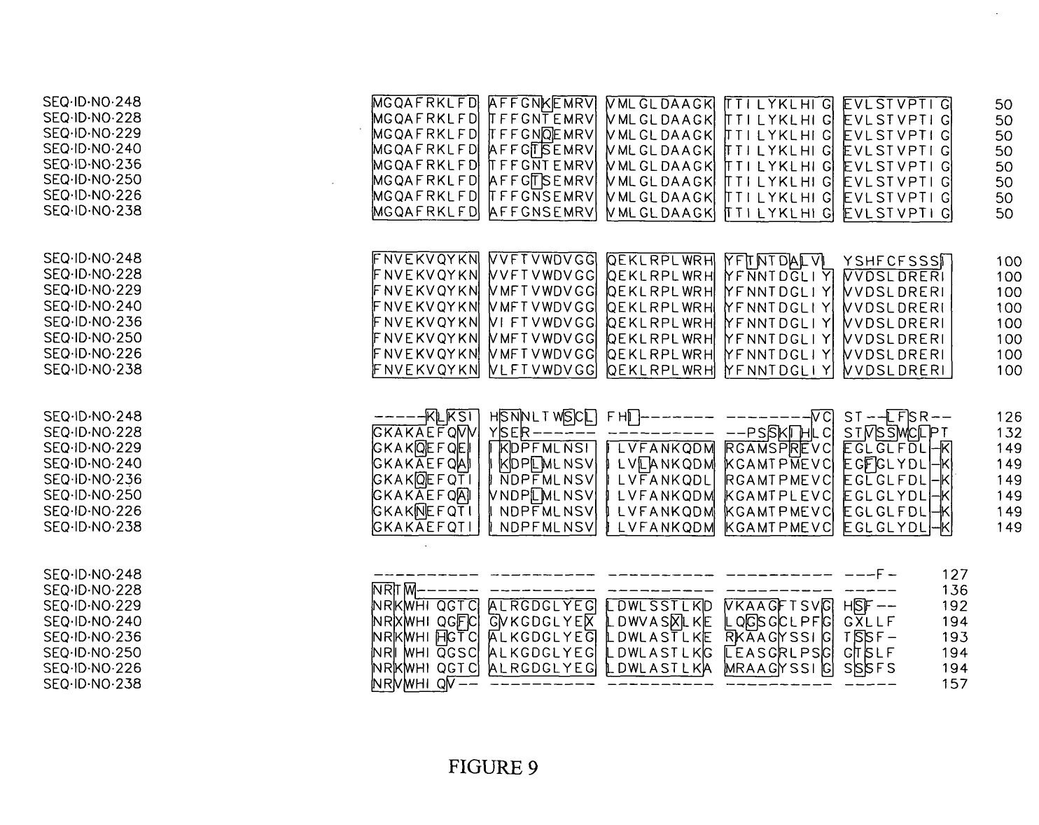

Amino acid sequences of homologs and/or orthologs of the polypeptide having the amino acid sequence set forth in SEQ ID NO:240 are provided in FIG. 9. The alignment in FIG. 9 provides the amino acid sequences of Ceres CLONE ID no. 1929841 (SEQ ID NO:226), Ceres ANNOT ID no. 1470444 (SEQ ID NO:228), GI no. 15237901 (SEQ ID NO:229), Ceres CLONE ID no. 707855 (SEQ ID NO:236), Ceres CLONE ID no. 757222 (SEQ ID NO:238), Ceres CLONE ID no. 1860083 (SEQ ID NO:248), and GI no. 41052966 (SEQ ID NO:250).

Other homologs and/or orthologs include Public GI no. 78709067 (SEQ ID NO: 99), Public GI no. 1184987 (SEQ ID NO:102), (SEQ ID NO:231), (SEQ ID NO:232), (SEQ ID NO:234), (SEQ ID NO:242), (SEQ ID NO:244), (SEQ ID NO:246), (SEQ ID NO:249), (SEQ ID NO:251), (SEQ ID NO:252), (SEQ ID NO:253), (SEQ ID NO:254), (SEQ ID NO:255), (SEQ ID NO:256) and (SEQ ID NO:257).

In some cases, a carotenoid-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:99, SEQ ID NO:102, SEQ ID NO:231, SEQ ID NO:232, SEQ ID NO:234, SEQ ID NO:242, SEQ ID NO:244, SEQ ID NO:246, SEQ ID NO:249, SEQ ID NO:251, SEQ ID NO:252, SEQ ID NO:253, SEQ ID NO:254, SEQ ID NO:255, SEQ ID NO:256, or SEQ ID NO:257.

A carotenoid-modulating polypeptide encoded by a recombinant nucleic acid can be a native carotenoid-modulating polypeptide, i.e., one or more additional copies of the coding sequence for a carotenoid-modulating polypeptide that is naturally present in the cell. Alternatively, a carotenoid-modulating polypeptide can be heterologous to the cell, e.g., a transgenic Lycopersicon plant can contain the coding sequence for a carotenoid-modulating polypeptide from a Glycine plant.

A carotenoid-modulating polypeptide can include additional amino acids that are not involved in carotenoid modulation, and thus can be longer than would otherwise be the case. For example, a carotenoid-modulating polypeptide can include an amino acid sequence that functions as a reporter. Such a carotenoid-modulating polypeptide can be a fusion protein in which a green fluorescent protein (GFP) polypeptide is fused to, e.g., SEQ ID NO: 80, or in which a yellow fluorescent protein (YFP) polypeptide is fused to, e.g., SEQ ID NO: 81. In some embodiments, a carotenoid-modulating polypeptide includes a purification tag, a chloroplast transit peptide, a mitochondrial transit peptide, an amyloplast transit peptide or a leader sequence added to the amino or carboxy terminus.

Carotenoid-modulating polypeptides suitable for use in the invention can be identified by analysis of nucleotide and polypeptide sequence alignments. For example, performing a query on a database of nucleotide or polypeptide sequences can identify homologs and/or orthologs of carotenoid-modulating polypeptides. Sequence analysis can involve BLAST, Reciprocal BLAST, or PSI-BLAST analysis of nonredundant databases using known carotenoid-modulating polypeptide amino acid sequences. Those polypeptides in the database that have greater than 40% sequence identity can be identified as candidates for further evaluation for suitability as a carotenoid-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in carotenoid-modulating polypeptides, e.g., conserved functional domains.

The identification of conserved regions in a template or subject polypeptide can facilitate production of variants of wild type carotenoid-modulating polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999).

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within carotenoid-modulating polypeptides. These conserved regions can be useful in identifying functionally similar (orthologous) carotenoid-modulating polypeptides.

In some instances, suitable carotenoid-modulating polypeptides can be synthesized on the basis of consensus functional domains and/or conserved regions in polypeptides that are homologous carotenoid-modulating polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Representative homologs and/or orthologs of carotenoid-modulating polypeptides are shown in FIGS. 1-9. Each Figure represents an alignment of the amino acid sequence of a carotenoid-modulating polypeptide with the amino acid sequences of corresponding homologs and/or orthologs. Amino acid sequences of carotenoid-modulating polypeptides and their corresponding homologs and/or orthologs have been aligned to identify conserved amino acids that contain frequently occurring amino acid residues at particular positions in the aligned sequences, as shown in FIGS. 1-9. A dash in an aligned sequence represents a gap, i.e., a lack of an amino acid at that position. Identical amino acids or conserved amino acid substitutions among aligned sequences are identified by boxes. Each conserved region contains a sequence of contiguous amino acid residues.

Useful polypeptides can be constructed based on the conserved regions in FIG. 1, FIG. 2, FIG. 3, FIG. 4, FIG. 5, FIG. 6. FIG. 7, FIG. 8, or FIG. 9. Such a polypeptide includes the conserved regions arranged in the order depicted in a Figure from amino-terminal end to carboxy-terminal end and has at least 80% sequence identity to an amino acid sequence corresponding to any one of SEQ ID NO's: SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:91, SEQ ID NO:92, SEQ ID NO:93, SEQ ID NO:104 SEQ ID NO:108, SEQ ID NO:128, SEQ ID NO:129, SEQ ID NO:131, SEQ ID NO:95, SEQ ID NO:96, SEQ ID NO:205, SEQ ID NO:210, SEQ ID NO:213, SEQ ID NO:215, SEQ ID NO:218, SEQ ID NO:222, SEQ ID NO:223, SEQ ID NO:89, SEQ ID NO:159, SEQ ID NO:161, SEQ ID NO:165, SEQ ID NO:172, SEQ ID NO:178, SEQ ID NO:180, SEQ ID NO:182, SEQ ID NO:183, SEQ ID NO:144, SEQ ID NO:133, SEQ ID NO:137, SEQ ID NO:146, SEQ ID NO:152, SEQ ID NO:154, SEQ ID NO:156, SEQ ID NO:157, SEQ ID NO:110, SEQ ID NO:294, SEQ ID NO:296, SEQ ID NO:306, SEQ ID NO:320, SEQ ID NO:322, SEQ ID NO:326, SEQ ID NO:331, SEQ ID NO:98, SEQ ID NO:259, SEQ ID NO:261, SEQ ID NO:262, SEQ ID NO:269, SEQ ID NO:271, SEQ ID NO:273, SEQ ID NO:281, SEQ ID NO:283, SEQ ID NO:240, SEQ ID NO:226, SEQ ID NO:228, SEQ ID NO:229, SEQ ID NO:236, SEQ ID NO:238, SEQ ID NO:248, SEQ ID NO:250, SEQ ID NO:185, SEQ ID NO:187, SEQ ID NO:189, SEQ ID NO:191, SEQ ID NO:193, SEQ ID NO:195, SEQ ID NO:196, SEQ ID NO:198, SEQ ID NO:199, SEQ ID NO:200, SEQ ID NO:201, SEQ ID NO:202, SEQ ID NO:203, SEQ ID NO:163), SEQ ID NO:167, SEQ ID NO:169, SEQ ID NO:170, SEQ ID NO:174, SEQ ID NO:176, SEQ ID NO:135, SEQ ID NO:139), SEQ ID NO:141, SEQ ID NO:143, SEQ ID NO:148, SEQ ID NO:150, SEQ ID NO:341, SEQ ID NO:343, SEQ ID NO:345, SEQ ID NO:347, SEQ ID NO:349, SEQ ID NO:351, SEQ ID NO:353, SEQ ID NO:354, SEQ ID NO:356, SEQ ID NO:357, SEQ ID NO:358, SEQ ID NO:359, SEQ ID NO:360, SEQ ID NO:361, SEQ ID NO:363, SEQ ID NO:365, SEQ ID NO:367, SEQ ID NO:368, SEQ ID NO:369, SEQ ID NO:370, SEQ ID NO:371, SEQ ID NO:292, SEQ ID NO:298, SEQ ID NO:300, SEQ ID NO:302, SEQ ID NO:304, SEQ ID NO:305, SEQ ID NO:307, SEQ ID NO:308, SEQ ID NO:309, SEQ ID NO:310, SEQ ID NO:311, SEQ ID NO:312, SEQ ID NO:313, SEQ ID NO:315, SEQ ID NO:316, SEQ ID NO:318, SEQ ID NO:324, SEQ ID NO:328, SEQ ID NO:329, SEQ ID NO:330, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID 106, SEQ ID NO: 206, SEQ ID NO: 208, SEQ ID NO: 212, SEQ ID NO: 216, SEQ ID NO: 220, SEQ ID NO: 224, SEQ ID NO: 100, SEQ ID NO: 101, SEQ ID NO: 264, SEQ ID NO: 265, SEQ ID NO: 267, SEQ ID NO: 275, SEQ ID NO: 277, SEQ ID NO: 279, SEQ ID NO: 282, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 99, SEQ ID NO:102, SEQ ID NO:231, SEQ ID NO:232, SEQ ID NO:234, SEQ ID NO:242, SEQ ID NO:244, SEQ ID NO:246, SEQ ID NO:249, SEQ ID NO:251, SEQ ID NO:252, SEQ ID NO:253, SEQ ID NO:254, SEQ ID NO:255, SEQ ID NO:256, or SEQ ID NO:257. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

In some embodiments, a carotenoid-modulating polypeptide is truncated at the amino- or carboxy-terminal end of a naturally occurring polypeptide. A truncated polypeptide may retain certain domains of the naturally occurring polypeptide while lacking others. Thus, length variants that are up to 5 amino acids shorter or longer typically exhibit the carotenoid-modulating activity of a truncated polypeptide. In some embodiments, a truncated polypeptide is a dominant negative polypeptide. SEQ ID NO: 89 sets forth the amino sequence of a carotenoid-modulating polypeptide that is truncated at the carboxyl end relative to the naturally occurring polypeptide. SEQ ID NO: 98 sets forth the amino sequence of a carotenoid-modulating polypeptide that is truncated at the carboxyl end relative to the naturally occurring polypeptide. Expression in a plant of such a truncated polypeptide confers a difference in the level of one or more carotenoids in a tissue of the plant as compared to the corresponding level in tissue of a control plant that does not comprise the truncation.

Conserved regions can be identified by homologous polypeptide sequence analysis as described above. The suitability of polypeptides for use as carotenoid-modulating polypeptides can be evaluated by functional complementation studies.

In some embodiments, useful polypeptides include those that fit a Hidden Markov Model based on the polypeptides set forth in any one of FIGS. 1-9. A Hidden Markov Model (HMM) is a statistical model of a consensus sequence for a group of homologous and/or orthologous polypeptides. See, Durbin et al., Biological Sequence Analysis Probabilistic Models of Proteins and Nucleic Acids, Cambridge University Press, Cambridge, UK (1998). An HMM is generated by the program HMMER 2.3.2 using the multiple sequence alignment of the group of homologous and/or orthologous sequences as input and the default program parameters. The multiple sequence alignment is generated by ProbCons (Do et al., Genome Res., 15(2):330-40 (2005)) version 1.11 using a set of default parameters: -c, —consistency REPS of 2; -ir, —iterative-refinement REPS of 100; -pre, —pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. The HMMER 2.3.2 package was released Oct. 3, 2003 under a GNU general public license, and is available from various sources on the World Wide Web such as hmmer.janelia.org, hmmer.wustl.edu, and fr.com/hmmer232/. Hmmbuild outputs the model as a text file.

The HMM for a group of homologous and/or orthologous polypeptides can be used to determine the likelihood that a candidate carotenoid-modulating polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 50, and often is higher.

The carotenoid modulating polypeptides described herein fit the indicated HMM with an HMM bit score greater than 50 (e.g., greater than 260, 280, 290, 300, 405, 410, 415, 420, 425, 430, 435, 440, 445, 450, 460, 470, 480, 490, 500, 520, 540, 560, 580, 600, 620, 640, 660, 680, 700, 720, 740, 760, or 780). In some embodiments, the HMM bit score of a carotenoid-modulating polypeptide described herein is about 80%, 81%, 82%, 83%, 84%, 85%, 86%, 87%, 88%, 89%, 90%, 91%, 92%, 93%, 94%, 95%, 96%, 97%, 98%, or 99% of the HMM bit score of a homologous and/or orthologous polypeptide provided in one of Tables 14-22. In some embodiments, a carotenoid modulating polypeptide fits the indicated HMM with an HMM bit score greater than 50, and has 80% or greater sequence identity (e.g., 80%, 85%, 90%, 95%, 97%, or 100% sequence identity) to an amino acid sequence shown in any one of FIGS. 1-9.

Polypeptides are shown in Table 14 that have HMM bit scores greater than 290 when fitted to an HMM generated from the amino acid sequences set forth in FIG. 1. Such polypeptides include Ceres Annot ID No. 1534144 (SEQ ID NO:185), Ceres Clone ID No. 463380 (SEQ ID NO: 195), and Public GI No. 1154352354 (SEQ ID NO: 200).

Polypeptides are shown in Table 15 that have HMM bit scores greater than 100 when fitted to an HMM generated from the amino acid sequences set forth in FIG. 2. Such polypeptides include Ceres Annot ID No. 1473516 (SEQ ID NO:345), Ceres clone ID No. 775387 (SEQ ID NO:365), and Public GI No. 12060388 (SEQ ID NO:368).

Polypeptides are shown in Table 16 that have HMM bit scores greater than 850 when fitted to an HMM generated from the amino acid sequences set forth in FIG. 3. Such polypeptides include Ceres Annot ID No. 1455046 (SEQ ID NO:106).

Polypeptides are shown in Table 17 that have HMM bit scores greater than 300 when fitted to an HMM generated from the amino acid sequences set forth in FIG. 4. Such polypeptides include Ceres Annot ID No. 1448769 (SEQ ID NO:208), Ceres clone ID No. 325641 (SEQ ID NO:222), and Public GI No. 115458786 (SEQ ID NO:224).

Polypeptides are shown in Table 18 that have HMM bit scores greater than 400 when fitted to an HMM generated from the amino acid sequences set forth in FIG. 5. Such polypeptides include SEQ ID NO: 163, SEQ ID NO: 169 and SEQ ID NO: 174.

Polypeptides are shown in Table 19 that have HMM bit scores greater than 600 when fitted to an HMM generated from the amino acid sequences set forth in FIG. 6. Such polypeptides include Ceres Annot ID No. 1511378 (SEQ ID NO:139), Ceres clone ID No. 942216 (SEQ ID NO:150), and Ceres Annot ID No. 1458137 (SEQ ID NO:141).

Polypeptides are shown in Table 20 that have HMM bit scores greater than 80 when fitted to an HMM generated from the amino acid sequences set forth in FIG. 7. Such polypeptides include Public GI No. 21264420 (SEQ ID NO:311), Ceres Clone ID No. 965028 (SEQ ID NO:318), and Ceres Clone ID No. 569593 (SEQ ID NO:328).

Polypeptides are shown in Table 21 that have HMM bit scores greater than 100 when fitted to an HMM generated from the amino acid sequences set forth in FIG. 8. Such polypeptides include SEQ ID NO:101, SEQ ID NO:264 and SEQ ID NO:285.

Polypeptides are shown in Table 22 that have HMM bit scores greater than 70 when fitted to an HMM generated from the amino acid sequences set forth in FIG. 9. Such polypeptides include Ceres clone ID No. 306269 (SEQ ID NO:244), Public GI No. 115483682 (SEQ ID NO:252), and Public GI No. 115443985 (SEQ ID NO:255).

### Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chema et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs can be used to transform plants or plant cells in order to modulate carotenoid levels. A recombinant nucleic acid construct can comprise a nucleic acid encoding a carotenoid-modulating polypeptide as described herein, operably linked to a regulatory region suitable for expressing the carotenoid-modulating polypeptide in the plant or cell. Thus, a nucleic acid can comprise a coding sequence that encodes any of the carotenoid-modulating polypeptides as set forth in SEQ ID NOs: 80-87, SEQ ID NO: 89, SEQ ID NOs: 91-93, SEQ ID NOs: 95-96, SEQ ID NOs: 98-102, SEQ ID NO: 104, SEQ ID NO:106, SEQ ID NO:108, SEQ ID NO: 110, SEQ ID NOs: 128-129, SEQ ID NO: 131; SEQ ID NO: 133, SEQ ID NO:135, SEQ ID NO:137, SEQ ID NO:139, SEQ ID NO:141, SEQ ID NOs:143-144, SEQ ID NO:146, SEQ ID NO:148, SEQ ID NO:150, SEQ ID NO:152, SEQ ID NO:154, SEQ ID NO:156, SEQ ID NO:157; SEQ ID NO: 159, SEQ ID NO: 161, SEQ ID NO: 163, SEQ ID NO: 165, SEQ ID NO: 167, SEQ ID NOs: 169-170, SEQ ID NO: 172, SEQ ID NO: 174, SEQ ID NO: 176, SEQ ID NO: 178, SEQ ID NO: SEQ ID NO: 180, SEQ ID NO: 182, SEQ ID NO: 183, SEQ ID NO: 185, SEQ ID NO: 187, SEQ ID NO: 189, SEQ ID NO: 191, SEQ ID NO: 193, SEQ ID NOs: 195-196, SEQ ID NOs: 199-203, SEQ ID NO: 205, SEQ ID NO: 206, SEQ ID NO: 208, SEQ ID NO: 210, SEQ ID NO: 212, SEQ ID NO: 213, SEQ ID NO: 215, SEQ ID NO: 216, SEQ ID NO: 218, SEQ ID NO: 220, SEQ ID NO: 222, SEQ ID NO: 223, SEQ ID NO: 224, SEQ ID NO:226, SEQ ID NO:228, SEQ ID NO:229, SEQ ID NO:231, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-257, SEQ ID NO:259, SEQ ID NO:261, SEQ ID NO:262, SEQ ID NO:264, SEQ ID NO:265, SEQ ID NO:267, SEQ ID NO:269, SEQ ID NO:271, SEQ ID NO:273, SEQ ID NO:275, SEQ ID NO:277, SEQ ID NO:279, SEQ ID NOs:281-290, SEQ ID NO:292, SEQ ID NO:294, SEQ ID NO:296, SEQ ID NO:298, SEQ ID NO:300, SEQ ID NO:302, SEQ ID NOs:304-313, SEQ ID NO:315, SEQ ID NO:316, SEQ ID NO:318, SEQ ID NO:320, SEQ ID NO:322, SEQ ID NO:324, SEQ ID NO:326, SEQ ID NOs:328-339, SEQ ID NO:341, SEQ ID NO:343, SEQ ID NO:345, SEQ ID NO:347, SEQ ID NO:349, SEQ ID NO:351, SEQ ID NO:353, SEQ ID NO:354, SEQ ID NOs:356-361, SEQ ID NO:363, SEQ ID NO:365, or SEQ ID NOs:367-371.

Examples of nucleic acids encoding carotenoid-modulating polypeptides are set forth in SEQ ID NO: 94, SEQ ID NO: 97, SEQ ID NO: 103, SEQ ID NO: 105, SEQ ID NO: 107, SEQ ID NO: 109, SEQ ID NO: 127, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 145, SEQ ID NO: 147, SEQ ID NO: 149, SEQ ID NO: 151, SEQ ID NO: 153, SEQ ID NO: 155, SEQ ID NO: 158, SEQ ID NO: 160, SEQ ID NO: 162, SEQ ID NO: 164, SEQ ID NO: 166, SEQ ID NO: 168, SEQ ID NO: 171, SEQ ID NO: 173, SEQ ID NO: 175, SEQ ID NO: 177, SEQ ID NO: 179, SEQ ID NO: 181, SEQ ID NO: 184, SEQ ID NO: 186, SEQ ID NO: 188, SEQ ID NO: 190, SEQ ID NO: 192, SEQ ID NO: 194, SEQ ID NO: 197, SEQ ID NO: 204, SEQ ID NO: 207, SEQ ID NO: 209, SEQ ID NO: 211, SEQ ID NO: 214, SEQ ID NO: 217, SEQ ID NO: 219, SEQ ID NO: 221, SEQ ID NO: 225, SEQ ID NO: 227, SEQ ID NO: 230, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 258, SEQ ID NO: 260, SEQ ID NO: 263, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 314, SEQ ID NO: 317, SEQ ID NO: 319, SEQ ID NO: 321, SEQ ID NO: 323, SEQ ID NO: 325, SEQ ID NO: 327, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 355, SEQ ID NO: 362, SEQ ID NO: 364, SEQ ID NO: 366 and SEQ ID NOs: 372-379.

In some cases, a recombinant nucleic acid construct can include a nucleic acid comprising less than the full-length coding sequence of a carotenoid-modulating polypeptide. For example, a recombinant nucleic acid construct can comprise a carotenoid-modulating nucleic acid having the nucleotide sequence set forth in SEQ ID NO: 89 or SEQ ID NO: 98. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given carotenoid-modulating polypeptide can be modified such that optimal expression in a particular plant species is obtained, using appropriate codon bias tables for that species.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### Regulatory Regions

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of promoters are described below. Some of the promoters indicated below as well as additional promoters are described in more detail in U.S. Patent Application Ser. Nos. 60/505,689; 60/518,075; 60/544,771; 60/558,869; 60/583,691; 60/583,609; 60/612,891; 60/619,181; 60/637,140; 60/757,544; 60/776,307; 10/950,321; 10/957,569; 11/058,689; 11/097,589; 11/172,703; 11/208,308; 11/233,726; 11/274,890; 11/360,017; 11/408,791; 11/414,142 PCT/US05/011105; PCT/US05/034308; PCT/US05/034343; PCT/US06/038236; PCT/US05/23639; PCT/US06/040572 and PCT/US07/62762. Nucleotide sequences of promoters are set forth in SEQ ID NOs: 1-78 and 111-126. It will be appreciated that a promoter may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO: 76), YP0144 (SEQ ID NO: 55), YP0190 (SEQ ID NO: 59), p13879 (SEQ ID NO: 75), YP0050 (SEQ ID NO: 35), p32449 (SEQ ID NO: 77), 21876 (SEQ ID NO: 1), YP0158 (SEQ ID NO: 57), YP0214 (SEQ ID NO: 61), YP0380 (SEQ ID NO: 70), PT0848 (SEQ ID NO: 26), and PT0633 (SEQ ID NO: 7) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128 (SEQ ID NO: 52), YP0275 (SEQ ID NO: 63), PT0625 (SEQ ID NO: 6), PT0660 (SEQ ID NO: 9), PT0683 (SEQ ID NO: 14), and PT0758 (SEQ ID NO: 22) promoters. Other root-preferential promoters include the PT0613 (SEQ ID NO: 5), PT0672 (SEQ ID NO: 11), PT0688 (SEQ ID NO: 15), and PT0837 (SEQ ID NO: 24) promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean a′ subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol., 13:5829-5842 (1993)), the beta-amylase promoter, and the barley hordein promoter. Other maturing endosperm promoters include the YP0092 (SEQ ID NO: 38), PT0676 (SEQ ID NO: 12), and PT0708 (SEQ ID NO: 17) promoters.

Ovary Tissue Promoters

Promoters that are active in ovary tissues such as the ovule wall and mesocarp can also be useful, e.g., a polygalacturonidase promoter, the banana TRX promoter, and the melon actin promoter. Examples of promoters that are active primarily in ovules include YP0007 (SEQ ID NO: 30), YP0111 (SEQ ID NO: 46), YP0092 (SEQ ID NO: 38), YP0103 (SEQ ID NO: 43), YP0028 (SEQ ID NO: 33), YP0121 (SEQ ID NO: 51), YP0008 (SEQ ID NO: 31), YP0039 (SEQ ID NO: 34), YP0115 (SEQ ID NO: 47), YP0119 (SEQ ID NO: 49), YP0120 (SEQ ID NO: 50), YP0374 (SEQ ID NO: 68), YP0396 (SEQ ID NO: 74), and PT0623 (SEQ ID NO: 126).

Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO: 34), YP0101 (SEQ ID NO: 41), YP0102 (SEQ ID NO: 42), YP0110 (SEQ ID NO: 45), YP0117 (SEQ ID NO: 48), YP0119 (SEQ ID NO: 49), YP0137 (SEQ ID NO: 53), DME, YP0285 (SEQ ID NO: 64), and YP0212 (SEQ ID NO: 60). Other promoters that may be useful include the following rice promoters: p530c10 (SEQ ID NO: 111), pOsFIE2-2 (SEQ ID NO: 112), pOsMEA (SEQ ID NO: 113), pOsYp102 (SEQ ID NO: 114), and pOsYp285 (SEQ ID NO: 115).

Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltpl) promoter (Plant Cell Rep (2001) 20:647-654), YP0097 (SEQ ID NO: 40), YP0107 (SEQ ID NO: 44), YP0088 (SEQ ID NO: 37), YP0143 (SEQ ID NO: 54), YP0156 (SEQ ID NO: 56), PT0650 (SEQ ID NO: 8), PT0695 (SEQ ID NO: 16), PT0723 (SEQ ID NO: 19), PT0838 (SEQ ID NO: 25), PT0879 (SEQ ID NO: 28), and PT0740 (SEQ ID NO: 20).

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO: 3), PT0668 (SEQ ID NO: 2), PT0886 (SEQ ID NO: 29), YP0144 (SEQ ID NO: 55), YP0380 (SEQ ID NO: 70), and PT0585 (SEQ ID NO: 4).

Vascular Tissue Promoters

Examples of promoters that have high or preferential activity in vascular bundles include YP0087 (SEQ ID NO: 118), YP0093 (SEQ ID NO: 119), YP0108 (SEQ ID NO: 120), YP0022 (SEQ ID NO: 141), and YP0080 (SEQ ID NO: 122). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)). Promoters having preferential activity in sieve, laticifer, and/or companion cells are also considered vascular tissue promoters.

Inducible Promoters

Inducible promoters confer transcription in response to external stimuli such as chemical agents or environmental stimuli. For example, inducible promoters can confer transcription in response to hormones such as gibberellic acid or ethylene, or in response to light or drought. Examples of drought-inducible promoters include YP0380 (SEQ ID NO: 70), PT0848 (SEQ ID NO: 26), YP0381 (SEQ ID NO: 71), YP0337 (SEQ ID NO: 66), PT0633 (SEQ ID NO: 7), YP0374 (SEQ ID NO: 68), PT0710 (SEQ ID NO: 18), YP0356 (SEQ ID NO: 67), YP0385 (SEQ ID NO: 73), YP0396 (SEQ ID NO: 74), YP0388 (SEQ ID NO: 124), YP0384 (SEQ ID NO: 72), PT0688 (SEQ ID NO: 15), YP0286 (SEQ ID NO: 65), YP0377 (SEQ ID NO: 69), PD1367 (SEQ ID NO: 78), PD0901 (SEQ ID NO: 125), and PD0898. Nitrogen-inducible promoters include PT0863 (SEQ ID NO: 27), PT0829 (SEQ ID NO: 23), PT0665 (SEQ ID NO:10), and PT0886 (SEQ ID NO:29). Examples of shade-inducible promoters include PRO924 (SEQ ID NO: 123), and PT0678 (SEQ ID NO: 13).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential such as PT0678 (SEQ ID NO: 13), tuber-preferential, parenchyma cell-preferential, and senescence-preferential promoters. Promoters designated YP0086 (SEQ ID NO: 36), YP0188 (SEQ ID NO: 58), YP0263 (SEQ ID NO: 62), PT0758 (SEQ ID NO: 22), PT0743 (SEQ ID NO: 21), PT0829 (SEQ ID NO: 23), YP0119 (SEQ ID NO: 32), and YP0096 (SEQ ID NO: 39), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding a carotenoid-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₃BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous carotenoid-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

Plant Species

The polynucleotides and vectors described herein can be used to transform a number of monocotyledonous and dicotyledonous plants and plant cell systems, including dicots such as alfalfa, almond, amaranth, apple, apricot, avocado, beans (including kidney beans, lima beans, dry beans, green beans), brazil nut, broccoli, cabbage, canola, carrot, cashew, castor bean, cherry, chick peas, chicory, chocolate, clover, cocoa, coffee, cotton, cottonseed, crambe, eucalyptus, flax, foxglove, grape, grapefruit, hazelnut, hemp, jatropha, jojoba, lemon, lentils, lettuce, linseed, macadamia nut, mango, melon (e.g., watermelon, cantaloupe), mustard, neem, olive, orange, peach, peanut, peach, pear, peas, pecan, pepper, pistachio, plum, poplar, poppy, potato, pumpkin, oilseed rape, quinoa, rapeseed (high erucic acid and canola), safflower, sesame, soaptree bark, soybean, spinach, strawberry, sugar beet, sunflower, sweet potatoes, tea, tomato, walnut, and yams, as well as monocots such as banana, barley, bluegrass, coconut, corn, date palm, fescue, field corn, garlic, millet, oat, oil palm, onion, palm kernel oil, pineapple, popcorn, rice, rye, ryegrass, sorghum, sudangrass, sugarcane, sweet corn, switchgrass, turf grasses, timothy, and wheat. Gymnosperms such as fir, pine, and spruce can also be suitable.

Thus, the methods and compositions described herein can be used with dicotyledonous plants belonging, for example, to the orders Apiales, Arecales, Aristochiales, Asterales, Batales, Campanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malpighiales, Malvales, Myricales, Mynales, Nymphaeales, Papaverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Lihales, Najadales, Orchidales, Pandanales, Poales, Restionales, Triuridales, Typhales, Zingiberales, and with plants belonging to Gymnospermae, e.g., Cycadales, Ephedrales, Ginkgoales, Gnetales, and Pinales.

The methods and compositions can be used over a broad range of plant species, including species from the dicot genera Acokanthera, Aconitum, Aesculus, Alangium, Alchomea, Alexa, Alseodaphne, Amaranthus, Ammodendron, Anabasis, Anacardium, Angophora, Anisodus, Apium, Apocynum, Arabidopsis, Arachis, Argemone, Asclepias, Atropa, Azadirachta, Beilschmiedia, Berberis, Bertholletia, Beta, Betula, Bixa, Bleekeria, Borago, Brassica, Calendula, Camellia, Camptotheca, Canarium, Cannabis, Capsicum, Carthamus, Carya, Catharanthus, Centella, Cephaelis, Chelidonium, Chenopodium, Chrysanthemum, Cicer, Cichorium, Cinchona, Cinnamomum, Cissampelos, Citrus, Citrullus, Cocculus, Cocos, Coffea, Cola, Convolvulus, Coptis, Corylus, Corymbia, Crambe, Crotalaria, Croton, Cucumis, Cucurbita, Cuphea, Cytisus, Datura, Daucus, Dendromecon, Dianthus, Dichroa, Digitalis, Dioscorea, Duguetia, Erythroxylum, Eschscholzia, Eucalyptus, Euphorbia, Euphoria, Ficus, Fragaria, Galega, Gelsemium, Glaucium, Glycine, Glycyrrhiza, Gossypium, Helianthus, Heliotropium, Hemsleya, Hevea, Hydrastis, Hyoscyamus, Jatropha, Juglans, Lactuca, Landolphia, Lavandula, Lens, Linum, Litsea, Lobelia, Luffa, Lupinus, Lycopersicon, Macadamia, Mahonia, Majorana, Malus, Mangifera, Manihot, Meconopsis, Medicago, Menispermum, Mentha, Micropus, Nicotiana, Ocimum, Olea, Origanum, Papaver, Parthenium, Persea, Petunia, Phaseolus, Physostigma, Pilocarpus, Pistacia, Pisum, Populus, Prunus, Psychotria, Pyrus, Quillaja, Rabdosia, Raphanus, Rhizocarya, Ricinus, Rosa, Rosmarinus, Rubus, Rubia, Salix, Salvia, Sanguinaria, Scopolia, Senecio, Sesamum, Simmondsia, Sinapis, Sinomenium, Solanum, Sophora, Spinacia, Stephania, Strophanthus, Strychnos, Tagetes, Theobroma, Thymus, Trifolium, Trigonella, Vaccinium, Vicia, Vigna, Vinca, and Vitis; and the monocot genera Agrostis, Allium, Ananas, Andropogon, Areca, Arundo, Asparagus, Avena, Cocos, Colchicum, Convallaria, Curcuma, Cynodon, Elaeis, Eragrostis, Festuca, Festulolium, Galanthus, Hemerocallis, Hordeum, Lemna, Lolium, Miscanthus, Musa, Oryza, Panicum, Pennisetum, Phalaris, Phleum, Phoenix, Poa, Ruscus, Saccharum, Secale, Sorghum, Triticosecale, Triticum, Veratrum, Zea, and Zoysia; and the gymnosperm genera Abies, Cephalotaxus, Cunninghamia, Ephedra, Picea, Pinus, Populus, and Pseudotsuga.

In some embodiments, a plant is a member of the species Brassica napus, Brassica rapa, Gossypium hirsutum, Glycine max, Lycopersicon esculentum, Musa paradisica, Lactuca sativa, Oryza sativa, Triticum aestivum and Zea mays.

### Methods of Inhibiting Expression of Carotenoid-Modulating Polypeptides

The polynucleotides and recombinant vectors described herein can be used to express or inhibit expression of a carotenoid-modulating polypeptide in a plant species of interest. The term “expression” refers to the process of converting genetic information of a polynucleotide into RNA through transcription, which is catalyzed by an enzyme, RNA polymerase, and into protein, through translation of mRNA on ribosomes. “Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

A number of nucleic-acid based methods, including antisense RNA, co-suppression, ribozyme directed RNA cleavage, and RNA interference (RNAi) can be used to inhibit protein expression in plants. Antisense technology is one well-known method. In this method, a nucleic acid segment from a gene to be repressed is cloned and operably linked to a promoter so that the antisense strand of RNA is transcribed. The recombinant vector is then transformed into plants, as described above, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

Thus, for example, an isolated nucleic acid provided herein can be an antisense nucleic acid to any of the aforementioned nucleic acids encoding a carotenoid-modulating polypeptide as set forth, for example, in SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 89, SEQ ID NO: 91, SEQ ID NO: 92, SEQ ID NO: 93, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 104, SEQ ID NO: 106, SEQ ID NO: 108 and SEQ ID NO: 110. A nucleic acid that decreases the level of a transcription or translation product of a gene encoding a carotenoid-modulating polypeptide is transcribed into an antisense nucleic acid that anneals to the sense coding sequence of the carotenoid-modulating polypeptide.

Constructs containing operably linked nucleic acid molecules in the sense orientation can also be used to inhibit the expression of a gene. The transcription product can be similar or identical to the sense coding sequence of a carotenoid-modulating polypeptide. The transcription product can also be unpolyadenylated, lack a 5′ cap structure, or contain an unsplicable intron. Methods of co-suppression using a full-length cDNA as well as a partial cDNA sequence are known in the art. See, e.g., U.S. Pat. No. 5,231,020.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

RNAi can also be used to inhibit the expression of a gene. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. A construct including a sequence that is transcribed into an interfering RNA is transformed into plants as described above. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

### Transgenic Plant Phenotypes

A transformed cell, callus, tissue, or plant can be identified and isolated by selecting or screening the engineered plant material for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known.

A population of transgenic plants can be screened and/or selected for those members of the population that have a desired trait or phenotype conferred by expression of the transgene. For example, a population of progeny of a single transformation event can be screened for those plants having a desired level of expression of a carotenoid-modulating polypeptide or nucleic acid. As an alternative, a population of plants comprising independent transformation events can be screened for those plants having a desired level of a carotenoid. Selection and/or screening can be carried out over one or more generations, which can be useful to identify those plants that have a desired trait, such as increased amounts of one or more carotenoids. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is exhibited by the plant.

Transgenic plants can have an altered phenotype as compared to a corresponding control plant that either lacks the transgene or does not express the transgene. A polypeptide can affect the phenotype of a plant (e.g., a transgenic plant) when expressed in the plant, e.g., at the appropriate time(s), in the appropriate tissue(s), or at the appropriate expression levels. Phenotypic effects can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

Thus, a transgenic plant or cell in which the expression of a carotenoid-modulating polypeptide is modulated can have modulated levels of one or more carotenoids relative to the carotenoid levels in a control plant that lacks or does not express the transgene. An amount of one or more of any individual carotenoid compounds can be modulated, e.g., increased or decreased, relative to a control plant not transgenic for the particular carotenoid-modulating polypeptide using the methods described herein. In certain cases, therefore, more than one carotenoid compound (e.g., two, three, four, five, six, seven, eight, nine, ten or even more carotenoid compounds) can have its amount modulated relative to a control plant or cell that is not transgenic for a carotenoid-modulating polypeptide described herein.

In some embodiments, a plant in which expression of a carotenoid-modulating polypeptide is modulated can have increased levels of one or more carotenoids in one or more tissues, e.g., aerial tissues, fruit tissues, root or tuber tissues, leaf tissues, stem tissues, or seeds. The increase in amount of one or more carotenoids can be restricted in some embodiments to particular tissues and/or organs, relative to other tissues and/or organs. For example, a transgenic plant can have an increased amount of a carotenoid in fruit tissue relative to leaf or root tissue.

The amount of one or more carotenoid compounds can be increased or decreased in a transgenic plant expressing a carotenoid-modulating polypeptide as described herein. An increase can be from about 2% to about 400% on a weight basis (e.g., a fresh or freeze dried weight basis) in such a transgenic plant compared to a corresponding control plant that lacks the recombinant nucleic acid encoding the carotenoid-modulating polypeptide. The carotenoid levels can be increased by at least 2 percent, e.g., 2, 5, 10, 15, 20, 25, 30, 35, 40, 45, 50, 55, 60, 65, 70, 75, 80, 85, 90 or more than 90 percent, as compared to the carotenoid levels in a corresponding control plant that does not express the transgene. In some embodiments, the increase is from about 5% to about 50%, or about 10% to about 40%, or about 50% to about 75%, or about 100% to about 200%, or about 200% to about 500% higher than the amount in a corresponding control cell that lacks the recombinant nucleic acid encoding a carotenoid-modulating polypeptide. In some embodiments, an increase can be from about 1.2-fold to about 10-fold, or about 1.2-fold to about 8-fold, or about 1.2-fold to about 6-fold, or about 1.2-fold to about 5-fold, or about 1.2-fold to about 4-fold, or about 1.2-fold to about 3-fold, or about 1.2-fold to about 2-fold, or about 1.3-fold to about 6-fold, or about 1.3-fold to about 5-fold, or about 1.3-fold to about 4-fold, or about 1.3-fold to about 3-fold, or about 1.3-fold to about 2.5-fold, or about 1.3-fold to about 2-fold, or about 1.3-fold to about 1.5-fold, or about 1.5-fold to about 6-fold, or about 1.5-fold to about 5-fold, or about 1.5-fold to about 4-fold, or about 1.5-fold to about 3-fold, or about 1.5-fold to about 2-fold, or about 2-fold to about 6-fold, or about 3-fold to about 4-fold, or about 3-fold to about 7-fold, or about 4-fold to about 8-fold, or about 5-fold to about 10-fold, higher than the amount in corresponding control cells or tissues that lack the recombinant nucleic acid encoding the carotenoid-modulating polypeptide.

In other embodiments, the carotenoid compound that is increased in transgenic plants or plant cells expressing a carotenoid-modulating polypeptide as described herein is either not produced or is not detectable in a corresponding control plant or plant cell that lacks the recombinant nucleic acid encoding the carotenoid-modulating polypeptide. Thus, in such embodiments, the increase in such a carotenoid compound is infinitely higher in a transgenic plant containing a recombinant nucleic acid encoding a carotenoid-modulating polypeptide than in a corresponding control plant or plant cell that lacks the recombinant nucleic acid encoding the carotenoid-modulating polypeptide. For example, in certain cases, a carotenoid-modulating polypeptide described herein may activate a biosynthetic pathway in a plant that is not normally activated or operational in a control plant, and one or more new carotenoids that were not previously produced in that plant species can be produced.

In some embodiments, a plant in which expression of a carotenoid-modulating polypeptide is modulated can have decreased levels of one or more carotenoids in one or more tissues, e.g., aerial tissues, fruit tissues, root or tuber tissues, leaf tissues, stem tissues, or seeds. The decrease in amount of one or more carotenoids can be restricted in some embodiments to particular tissues and/or organs, relative to other tissues and/or organs. For example, a transgenic plant can have a decreased amount of a carotenoid in fruit tissue relative to leaf or root tissue.

The amount of one or more carotenoid compounds can be increased or decreased in a transgenic plant expressing a carotenoid-modulating polypeptide as described herein. A decrease can be from about 2% to about 80% on a weight basis (e.g., a fresh or freeze dried weight basis) in such a transgenic plant compared to a corresponding control plant that lacks the recombinant nucleic acid encoding the carotenoid-modulating polypeptide. The carotenoid levels can be decreased by at least 2 percent, e.g., 2, 5, 10, 15, 20, 25, 30, 35, 40, 45, 50, 55, 60, 65, 70, 75, 80, or more than 80 percent, as compared to the carotenoid levels in a corresponding control plant that does not express the transgene. In some embodiments, the decrease is from about 5% to about 50%, or about 10% to about 40%, or about 50% to about 75%, or about 60% to about 80% lower than the amount in a corresponding control cell that lacks the recombinant nucleic acid encoding a carotenoid-modulating polypeptide. In some embodiments, the carotenoid level is from about 0.2-fold to about 0.9-fold, or from about 0.3-fold to about 0.8-fold, or from about 0.5-fold to about 0.9-fold or from about 0.4-fold to about 0.9 fold, or from about 0.4-fold to about 0.7-fold lower than the amount in a corresponding control cell that lacks the recombinant nucleic acid encoding a carotenoid-modulating polypeptide.

In certain embodiments, a carotenoid compound that is decreased in transgenic plants or plant cells expressing a carotenoid-modulating polypeptide as described herein is decreased to an undetectable level as compared to the level in a corresponding control plant or plant cell that lacks the recombinant nucleic acid encoding the carotenoid-modulating polypeptide.

In some embodiments, the amounts of two or more carotenoids are increased and/or decreased, e.g., the amounts of two, three, four, five, six, seven, eight, nine, ten (or more) carotenoid compounds are independently increased and/or decreased.

The amount of a carotenoid compound can be determined by known techniques, e.g., by extraction of carotenoid compounds from plants or plant tissues followed by gas chromatography-mass spectrometry (GC-MS) or liquid chromatography-mass spectrometry (LC-MS). If desired, the structure of the carotenoid compound can be confirmed by GC-MS, LC-MS, nuclear magnetic resonance and/or other known techniques.

Typically, a difference (e.g., an increase) in the amount of any individual carotenoid compound in a transgenic plant or cell relative to a control plant or cell is considered statistically significant at p<0.05 with an appropriate parametric or non-parametric statistic, e.g., Chi-square test, Student's t-test, Mann-Whitney test, or F-test. In some embodiments, a difference in the amount of any individual carotenoid compound is statistically significant at p<0.01, p<0.005, or p<0.001. A statistically significant difference in, for example, the amount of any individual carotenoid compound in a transgenic plant compared to the amount in cells of a control plant indicates that (1) the recombinant nucleic acid present in the transgenic plant results in altered levels of one or more carotenoid compounds and/or (2) the recombinant nucleic acid warrants further study as a candidate for altering the amount of a carotenoid compound in a plant.

Increases in carotenoids in plants can provide increased yields of carotenoids extracted from the plant tissues and increased nutritional content in foodstuffs and animal feed produced from the plant tissues. Decreases in carotenoids in plants can be useful in situations where altering the color or appearance of a plant is desired.

Information that the polypeptides disclosed herein can modulate carotenoid content can be useful in breeding of crop plants. Based on the effect of disclosed polypeptides on carotenoid content, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in carotenoid content. Those polymorphisms that are correlated with an alteration in carotenoid content can be incorporated into a marker assisted breeding program to facilitate the development of lines that have a desired alteration in carotenoid content. Typically, a polymorphism identified in such a manner is used with polymorphisms at other loci that are also correlated with a desired alteration in carotenoid content.

### Methods of Producing Carotenoids

Also provided herein are methods for producing one or more carotenoids. Exemplary carotenoids include, without limitation, phytoene, α-carotene, cis-β-carotene, trans-β-carotene, γ-carotene, δ-carotene, ζ-carotene, zeacarotene, lycopene, lutein, zeaxanthin, antheraxanthin, astaxanthin, bixin, capsanthin, fucoxanthin, β-cryptoxanthin, neoxanthin, and violaxanthin. Such methods can include growing a plant cell that includes a nucleic acid encoding a carotenoid-modulating polypeptide as described herein, under conditions effective for the expression of the carotenoid-modulating polypeptide. Also provided herein are methods for modulating (e.g., altering, increasing, or decreasing) the amounts of one or more carotenoids in a plant cell. The methods can include growing a plant cell as described above, i.e., a plant cell that includes a nucleic acid encoding a carotenoid-modulating polypeptide as described herein. The one or more carotenoids produced by these methods can be novel carotenoids, e.g., not normally produced in a wild-type plant cell.

The methods can further include the step of recovering one or more carotenoids from the cells. For example, plant cells known or suspected of producing one or more carotenoids can be subjected to fractionation to recover a desired carotenoid. Typically, fractionation is guided by in vitro assay of fractions. In some instances, cells containing one or more carotenoid compounds can be separated from cells not containing, or containing lower amounts of the carotenoid, in order to enrich for cells or cell types that contain the desired compound(s). A number of methods for separating particular cell types or tissues are known to those having ordinary skill in the art.

Fractionation can be carried out by techniques known in the art. For example, plant tissues or organs can be extracted with 100% MeOH to give a crude oil which is partitioned between several solvents in a conventional manner. As an alternative, fractionation can be carried out on silica gel columns using methylene chloride and ethyl acetate/hexane solvents.

In some embodiments, a fractionated or unfractionated plant tissue or organ is subjected to mass spectrometry in order to identify and/or confirm the presence of a desired carotenoid(s). See, e.g., WO 02/37111. In some embodiments, electrospray ionization (ESI) mass spectrometry can be used. In other embodiments, atmospheric pressure chemical ionization (APCI) mass spectrometry is used. If it is desired to identify higher molecular weight molecules in an extract, matrix-assisted laser desorption/ionization time-of-flight (MALDI-TOF) mass spectrometry can be useful.

Articles of Manufacture

Transgenic plants provided herein have particular uses in the agricultural and nutritional industries. For example, transgenic plants described herein can be used to make food products such as fresh, frozen, or canned vegetables and fruits. Suitable plants with which to make such products include, without limitation, tomatoes, lettuce, bananas, rice, corn, wheat, canola, cotton, soybean, mangos, melons, strawberries, peaches, apricots, oranges, papayas, corn, sweet potatoes, carrots, pumpkin, peppers, kale and spinach. Transgenic plants described herein can also be used to make processed food products such as tomato sauce, ketchup, jellies, and jams from the above fruits and vegetables. Such products are useful to provide increased amounts of carotenoids in a human diet.

Transgenic plants described herein can also be used as a source of animal feeds. Suitable plants with which to make such products include maize, wheat, soybean, cotton, canola, sunflower, safflower, oats, barley, and millet.

Transgenic plants or tissues from transgenic plants described herein can also be used as a source from which to extract carotenoids, using techniques known in the art. The resulting extract can be included in nutritional supplements as well as processed food products, e.g., snack products, frozen entrees, vegetable oils, breakfast cereals, and baby foods. The extracted carotenoids can also be used as starting materials for making fragrance chemicals for perfumes and other cosmetics.

Seeds of transgenic plants described herein can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package. The package label may indicate that the seed herein incorporates transgenes that provide increased amounts of one or more carotenoids in one or more tissues of plants grown from such seeds.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Transgenic Plants

The following symbols are used in the Examples: T1: first generation transformant; T2: second generation, progeny of self-pollinated T1 plants; T3: third generation, progeny of self-pollinated T2 plants. Independent transformations are referred to as events.

The following nucleic acids were isolated from Arabidopsis thaliana plants. Ceres Clone 34553 (SEQ ID NO: 79) is a cDNA clone that is predicted to encode a bZIP transcription factor polypeptide. Ceres Clone 21863 (SEQ ID NO: 88) is a cDNA clone that is predicted to encode an amino acid ubiquitin protein ligase. Ceres Clone 968026 (SEQ ID NO: 90) is a cDNA clone that is predicted to encode an amino acid response regulator 6 protein. Ceres Clone 641355 (SEQ ID NO: 109) is a cDNA clone that is predicted to encode an EREBP DNA-binding domain containing protein. Ceres Clone 13930 (SEQ ID NO: 103) is a cDNA clone that is predicted to encode a TPR domain protein. Ceres Clone 34589 (SEQ ID NO: 94) is a cDNA clone that is predicted to encode a Myb-like DNA binding domain-containing polypeptide. Ceres Clone 316638 (SEQ ID NO: 97) is a cDNA clone that is predicted to encode an ADP ribosylation factor polypeptide.

Each isolated nucleic acid described above was cloned, using standard molecular biology techniques, into a Ti plasmid vector, CRS 338, which encodes a selectable marker gene, phosphinothricin acetyltransferase, that confers Finale® resistance to transformed plants. Constructs were made using the CRS 338 vector that contained either the cDNA Ceres Clone 34553 (SEQ ID NO: 79), the cDNA Ceres Clone 21863 (SEQ ID NO: 88), Ceres Clone 968026 (SEQ ID NO: 90) Ceres Clone 641355 (SEQ ID NO: 109), Ceres Clone 13930 (SEQ ID NO: 103), or Ceres Clone 34589 (SEQ ID NO: 94), Ceres Clone 316638 (SEQ ID NO: 97) operably linked in the sense orientation relative to a CaMV 35S constitutive promoter.

The constructs were introduced separately into Arabidopsis ecotype Wassilewskija (WS-2) plants by the floral dip method essentially as described in Bechtold, N. et al., C.R. Acad. Sci. Paris, 316:1194-1199 (1993). Transgenic Arabidopsis plants containing Ceres Clone 34553 (SEQ ID NO: 79), Ceres Clone 21863 (SEQ ID NO: 88), Ceres Clone 968026 (SEQ ID NO: 90), Ceres Clone 641355 (SEQ ID NO: 109), Ceres Clone 13930 (SEQ ID NO: 103), or Ceres Clone 34589 (SEQ ID NO: 94), Ceres Clone 316638 (SEQ ID NO: 97) were designated ME03037, ME04864, ME05220, ME05058, ME06485, ME01130, or ME12093 Events, respectively. The presence of the vector DNA in each of these events was confirmed by screening the T1 plants for Finale® resistance. Control plants were transformed with the CRS vector lacking the cDNA insert.

T1 plants from ME03037, ME04864 and ME05220 events were evaluated for morphology and development. Morphology and development for 9 out of 10 plants from ME03037 events were similar to that of control plants; Event ME03037-01 had a few leaves which appeared lanceolate in shape. Morphology and development for 9 out of 10 plants from ME04864 events were similar to that of control plants; Event ME04864-01 had fewer leaves which appeared lanceolate in shape. Morphology and development for 9 out of 10 plants from ME05220 events were similar to that of control plants; Event ME05220-01 had fewer leaves which appeared lanceolate in shape.

T1 seeds were germinated and allowed to self-pollinate. T2 seeds were collected and a portion was germinated, allowed to self-pollinate, and T3 seeds were collected.

### Example 2

### Analysis of Carotenoids in Transgenic Arabidopsis Aerial Tissues

For carotenoid and chlorophyll profiling, aerial tissues from four Finale®-resistant plants of each event were pooled, frozen in liquid nitrogen and lyophilized. Tissues were then crushed into a fine powder and stored at −80° C.

For analysis of samples described in Example 3, 20 mg of lyophilized, ground aerial tissue was extracted in a 1:1 mixture of methanol/50 mM Tris-HCl (pH 7.5, 1 mM NaCl) in the dark at 4° C. Extracts were portioned using dichloromethane and the non-polar phase was subsequently removed and dried in vacuo. The dried non-polar phase was resuspended in ethyl acetate and analyzed using HPLC-PDA utilizing a methanol/tert-butyl methyl ether mobile phase and a C30 column. Chromatographic peaks were identified by either standard, UV spectrum or both. For data analysis, peak areas were calculated by the chromatography software and validated manually. Resulting areas were exported to Microsoft Excel where additional data analysis was performed.

For analysis of samples described in Examples 4, 5, 6, 7, 8, and 9, about 30 mg±3 mg of ground tissue was extracted with 1.5 mL of a 4:3 v/v of ethanol and hexane containing 0.05% w/v butylatedhydroxy toluene. Sixty μL of a solution containing 1 μg/μL of trans-crocetin in ethanol was added as an internal standard. The mixture was mixed by inversion on an orbital shaker for 45 minutes at 4° C. in the dark. Care was taken not to expose the extract to heat or light. The extract was, decanted into a syringe and filtered through a 0.22 micron filter into an amber LC-MS vial. The extract was analyzed for carotenoid content using a Waters 2795 Alliance system with a 996 PDA Detector, a Micromass ZMD single quadrupole mass spectrometer, and an atmospheric pressure chemical ionization probe (Waters Corp., Milford, Mass.). Separation of molecules was accomplished using a Luna C₁₈(2) 4.6×150 mm column (Phenomenex, Torrance, Calif.). Carotenoid compounds were identified based on spectral characteristics and comparison to reference standards and published retention times.

### Example 3

### Analysis of ME03037 Events

Carotenoid levels in aerial tissues from four ME03037 T2 events and four ME03037 T3 events, each containing Ceres Clone 34553 (SEQ ID NO: 79) were analyzed as described in Example 2. Increases in β-carotene content relative to the transgenic control plants were observed in ME03037 events as described below.

The cis-β-carotene content in T2 aerial tissue from two events of ME03037 was significantly increased relative to the cis-β-carotene content in aerial tissues in transgenic control plants. As shown in Table 1, cis-β-carotene levels in aerial tissues from events ME03037-02 and ME03037-03 were increased by 21% and 30%, respectively, compared to the cis-β-carotene levels in transgenic control plants.

The cis-β-carotene content in T3 aerial tissue from two events of ME03037 was significantly increased relative to the cis-β-carotene content in aerial tissues in transgenic control plants. As shown in Table 1, cis-β-carotene levels in aerial tissues from events ME03037-02 and ME03037-03 were increased by 41% and 28%, respectively, compared to the cis-β-carotene levels in transgenic control plants.

TABLE 1
cis-β-Carotene Levels (% Control) in ME03037 T₂ and T₃ Generations

| | ME03037-02 | ME03037-03 | ME03037-04 | ME03037-05 | Control |
|---|---|---|---|---|---|
| T₂ | 121 ± 11 | 130 ± 4 | 117 ± 7 | 104 ± 19 | 100 ± 9 |
| p-value | 0.03 | p < 0.01 | 0.05 | 0.88 | NA |
| T₃ | 141 ± 12 | 128 ± 9 | 113 ± 2 | 110 ± 12 | 100 ± 11 |
| p-value | 0.03 | 0.03 | p < 0.01 | 0.33 | NA |

N/A = not applicable
p-values were calculated using a Student's t-test

Aerial tissues from ME03037 T2 and T3 events were also increased in trans-β-carotene content. The combined area under the peaks corresponding to cis-β-carotene and trans-β-carotene was increased in extracts fromT2 Events ME03037-02 and ME03037-03 relative to the corresponding peak are in extracts from non-transgenic controls, although the increase was not statistically significant (p>0.05). The combined area under the peaks corresponding to cis-β-carotene and trans-β-carotene was significantly increased in extracts from T3 Events ME03037-02 and ME03037-03 relative to the corresponding area in extracts from non-transgenic controls.

There were no observable or statistically significant differences between T2 ME03037 and control plants in germination, onset of flowering, rosette area, fertility, and general morphology/architecture.

### Example 4

### Analysis of ME04864 Events

Carotenoid levels in aerial tissues from five ME04864 T2 events and five ME04864 T3 events, each containing Ceres Clone 21863 (SEQ ID NO: 88) were analyzed as described in Example 2.

The trans-lutein content in T2 aerial tissue from two events of ME04864 was significantly increased relative to the trans-lutein content in aerial tissues in non-transgenic control plants. As shown in Table 2, trans-lutein levels in aerial tissues from events ME04864-01 and ME04864-02 were increased by 71% and 66%, respectively, relative to trans-lutein levels in non-transgenic control plants. The standard error for this experiment was calculated based on three replicates for the experimental events and 12 replicates for the transgenic controls. Each replicate represented an extraction of pooled tissues from four individual plants.

The trans-lutein content in T3 aerial tissue from two events of ME04864 was significantly increased relative to the trans-lutein content in aerial tissues in non-transgenic control plants. Trans-lutein levels in aerial tissues from events ME04864-01 and ME04864-02 were increased by 32% and 23%, respectively, relative to trans-lutein levels in non-transgenic control plants. The standard error for this experiment was calculated based on three replicates for the experimental events and 12 replicates for the transgenic controls.

To confirm that the trans-lutein levels for all samples were within the linear range of analysis for the assay, a standard LC-MS calibration curve was prepared using a trans-lutein standard. Concentrations of the standard were 0.001, 0.01, 0.1, 1.0, and 10 mg/L; the R² value for the curve was 0.998.

TABLE 2
Trans-lutein content (% control) in ME04864 aerial tissue in T2 and T3 generations

| | ME04864-01 | ME04864-02 | ME04864-03 | ME04864-04 | ME04864-05 | Control |
|---|---|---|---|---|---|---|
| T2 | 171 ± 15 | 166 ± 10 | 181 ± 20 | 140 ± 15 | 124 ± 6 | 100 ± 6 |
| p-value* | 0.03 | 0.01 | 0.04 | 0.10 | 0.02 | N/A |
| T3 | 132 ± 7 | 123 ± 1 | 111 ± 4 | 115 ± 14 | 113 ± 0 | 100 ± 6 |
| p-value* | 0.01 | <0.01 | 0.19 | 0.40 | 0.05 | N/A |

N/A = not applicable
*p-values were calculated using a Student's t-test

The β-carotene levels in aerial tissues of T2 and T3 ME04864 events are shown in Table 3. As used in this example, β-carotene includes both isomers of β-carotene, i.e., cis-β-carotene and trans-β-carotene. The β-carotene content in T3 aerial tissue from two events of ME04864 was significantly increased relative to the β-carotene content in aerial tissues in transgenic control plants. β-carotene levels in aerial tissues from events ME04864-04 and ME04864-05 were increased by 55% and 43%, respectively, relative to β-carotene levels in transgenic control plants.

TABLE 3
β-carotene levels (% control) in ME04864 aerial tissue in T₂ and T₃ generations

| | ME04864-01 | ME04864-02 | ME04864-03 | ME04864-04 | ME04864-05 | Control |
|---|---|---|---|---|---|---|
| T2 | 123 ± 36 | 128 ± 34 | 151 ± 35 | 178 ± 48 | 140 ± 28 | 100 ± 7 |
| p-value* | 0.60 | 0.50 | 0.27 | 0.24 | 0.28 | N/A |
| T3 | 121 ± 46 | 106 ± 15 | 114 ± 37 | 155 ± 10 | 143 ± 9 | 100 ± 7 |
| p-value* | 0.70 | 0.82 | 0.78 | 0.01 | 0.01 | N/A |

N/A = not applicable
*p-values were calculated using a Student's t-test

There were no observable or statistically significant differences between T2 ME04864-02, ME04864-03, and control plants in germination, onset of flowering, rosette area, and general morphology/architecture. Finale® resistance for the T2 Event ME04864-01 segregated at a 3:1 ratio of resistance:sensitivity, consistent with the presence of a single insert. Finale® resistance for the T2 Event ME04864-02 segregated at a 15:1 ratio of resistance:sensitivity, consistent with the presence of two inserts.

### Example 5

### Analysis of ME05220 Events

Carotenoid levels in aerial tissues from five ME05220 T2 events and five ME05220 T3 events, each containing Ceres Clone 968026 (SEQ ID NO: 90) were analyzed as described in Example 2.

The ζ-carotene content in T2 aerial tissue from two events of ME05220 was significantly increased relative to the ζ-carotene content in aerial tissues in transgenic control plants. As shown in Table 4, ζ-carotene levels in aerial tissues from events ME05220-01 and ME05220-04 were increased by 102% and 138%, respectively, relative to ζ-carotene levels in transgenic control plants. The standard error for this experiment was calculated based on three replicates for the experimental events and 12 replicates for the transgenic controls. Each replicate represented an extraction of pooled tissues from four individual plants.

The ζ-carotene content in T3 aerial tissue from two events of ME05220 was significantly increased relative to the ζ-carotene content in aerial tissues in transgenic control plants. ζ-carotene levels in aerial tissues from events ME05220-01 and ME05220-04 were increased by 92% and 102%, respectively, relative to ζ-carotene levels in transgenic control plants

The standard error for this experiment was calculated based on three replicates for the experimental events and 12 replicates for the transgenic controls.

TABLE 4
ζ-carotene content (% control) in ME05220 T2 and T3 aerial tissue

| | ME05220-01 | ME05220-02 | ME05220-03 | ME05220-04 | ME05220-05 | Control |
|---|---|---|---|---|---|---|
| T2 | 202 ± 23 | 485 ± 154 | 129 ± 20 | 238 ± 104 | 140 ± 29 | 100 ± 9 |
| p-value* | 0.02 | <0.01 | 0.38 | 0.04 | 0.37 | N/A |
| T3 | 192 ± 37 | 105 ± 22 | 80 ± 20 | 202 ± 26 | 65 ± 18 | 100 ± 9 |
| p-value* | 0.03 | 0.98 | 0.32 | 0.02 | 0.17 | N/A |

N/A = not applicable
*p-values were calculated using a Student's t-test

There were no observable or statistically significant differences between T2 ME05220-01, ME05220-04, and control plants in germination, onset of flowering, rosette area, and general morphology/architecture. Finale® resistance for the T2 ME05220-01 and ME05220-04 events segregated at a 3:1 ratio of resistance:sensitivity, consistent with the presence of a single insert.

### Example 6

### Analysis of ME05058 Events

Carotenoid levels in aerial tissues from five ME05058 T2 events and four T3 events, each containing Ceres Clone 641355 (SEQ ID NO: 109), were analyzed as described in Example 2. To confirm that the trans-lutein levels for all samples were within the linear range of analysis for this assay, a standard LC-MS calibration curve was prepared using a trans-lutein standard according to the method described in Example 3.

As shown in Tables 5-7, the trans-lutein, neoxanthin, and violoxanthin contents in T2 aerial tissue from two events of ME05058 was significantly increased relative to the corresponding amounts in aerial tissues in transgenic control plants. For example, as shown in Table 5, trans-lutein levels in aerial tissues from T2 events ME05058-01 and ME05058-02 were increased by 216% and by 111%, respectively, relative to trans-lutein levels in transgenic control plants. Trans-lutein levels in aerial tissues from T3 events ME05058-01 and ME05058-02 were increased by 128% and by 67%, respectively, relative to trans-lutein levels in transgenic control plants. The standard error for this experiment was calculated based on three replicates for the experimental events and 12 replicates for the transgenic controls. Each replicate represented an extraction of pooled tissues from four individual plants.

TABLE 5
Trans-Lutein Increase (Fold-Increase) In ME05058 T₂ and T₃ Generation

| | ME05058-01 | ME05058-02 | ME05058-03 | ME05058-04 | ME05058-05 | Control |
|---|---|---|---|---|---|---|
| T₂ | 3.16 ± 0.13 | 2.11 ± 0.04 | 1.62 ± 0.13 | 1.23 ± 0.04 | 1.35 ± 0.04 | 1.00 ± 0.36 |
| p-value | 0.04 | p < 0.01 | 0.08 | 0.18 | 0.12 | N/A |
| T₃ | 2.28 ± 0.12 | 1.67 ± 0.08 | 1.69 ± 0.02 | Not | 1.82 ± 0.09 | 1.00 ± 0.07 |
| | | | | determined | | |
| p-value | 0.02 | 0.03 | p < 0.01 | Not | 0.02 | N/A |
| | | | | determined | | |

*p-values were calculated using a Student's t-test

As shown in Table 6, neoxanthin levels in aerial tissues from T2 events ME05058-01 and ME05058-02 were increased by 155% and by 133%, respectively, relative to neoxanthin levels in transgenic control plants. Neoxanthin levels in aerial tissues from T3 events ME05058-01 and ME05058-02 were increased by 112% and by 67%, respectively, relative to neoxanthin levels in transgenic control plants.

TABLE 6
Neoxanthin Increase (Fold Increase) in ME05058 T₂ and T₃ Generation

| | ME05058-01 | ME05058-02 | ME05058-03 | ME05058-04 | ME05058-05 | Control |
|---|---|---|---|---|---|---|
| T₂ | 2.55 ± 0.01 | 2.33 ± 0.01 | 2.32 ± 0.01 | 1.62 ± 0.01 | 1.47 ± 0.01 | 1.00 ± 0.33 |
| p-value | 0.02 | 0.04 | 0.21 | 0.23 | 0.12 | N/A |
| T₃ | 2.12 ± 0.01 | 1.67 ± 0.01 | 1.50 ± 0.01 | Not | 1.50 ± 0.01 | 1.00 ± 0.16 |
| | | | | determined | | |
| p-value | p < 0.01 | p < 0.01 | 0.18 | Not | 0.04 | N/A |
| | | | | determined | | |

*p-values were calculated using a Student's t-test

As shown in Table 7, violaxanthin levels in aerial tissues from T2 events ME05058-01 and ME05058-02 were increased by 188% and by 128%, respectively, relative to violaxanthin levels in transgenic control plants. Violaxanthin levels in aerial tissues from T3 events ME05058-01 and ME05058-02 were increased by 166% and by 45%, respectively, relative to violaxanthin levels in transgenic control plants.

TABLE 7
Violaxanthin Increase (Fold Increase) in ME05058 T₂ and T₃ Generation

| | ME05058-01 | ME05058-02 | ME05058-03 | ME05058-04 | ME05058-05 | Control |
|---|---|---|---|---|---|---|
| T₂ | 2.88 ± 0.01 | 2.28 ± 0.01 | 2.20 ± 0.01 | 1.49 ± 0.01 | 1.72 ± 0.01 | 1.00 ± 0.23 |
| p-value | 0.05 | 0.04 | 0.23 | 0.24 | 0.12 | N/A |
| T₃ | 2.66 ± 0.01 | 1.45 ± 0.01 | 1.74 ± 0.01 | Not | 2.03 ± 0.01 | 1.00 ± 0.07 |
| | | | | determined | | |
| p-value | p < 0.01 | 0.05 | 0.02 | Not | p < 0.01 | N/A |
| | | | | determined | | |

*p-values were calculated using a Student's t-test

Chlorophyll a and b levels were also analyzed from the ME05058 events. As shown in Table 8, the chlorophyll a level was significantly increased in aerial tissues from four T2 events (ME05058-01, -02, -04, and -05), relative to transgenic control plants. The chlorophyll a level was significantly increased in aerial tissues from one T3 event (ME05058-03), relative to transgenic control plants.

As shown in Table 9, chlorophyll b levels were significantly increased in aerial tissues from two T2 (ME05058-01 and -02) events, relative to transgenic control plants. Chlorophyll b levels were significantly increased in aerial tissues from one T3 event (ME05058-05), relative to transgenic control plants.

TABLE 8
Chlorophyll a Increase (Fold Increase) in ME05058 T₂ and T₃ Generations

| | ME05058-01 | ME05058-02 | ME05058-03 | ME05058-04 | ME05058-05 | Control |
|---|---|---|---|---|---|---|
| T₂ | 1.97 ± 0.09 | 1.88 ± 0.07 | 1.42 ± 0.08 | 1.31 ± 0.02 | 1.43 ± 0.03 | 1.00 ± 0.42 |
| p-value | 0.04 | 0.02 | 0.13 | 0.03 | 0.01 | N/A |
| T₃ | 1.39 ± 0.03 | 1.12 ± 0.01 | 1.41 ± 0.01 | Not | 1.35 ± 0.05 | 1.00 ± 0.15 |
| | | | | determined | | |
| p-value | 0.25 | 0.06 | 0.01 | Not | 0.21 | N/A |
| | | | | determined | | |

*p-values were calculated using a Student's t-test

TABLE 9
Chlorophyll b Increase (Fold Increase) in ME05058 T₂ and T₃ Generations

| | ME05058-01 | ME05058-02 | ME05058-03 | ME05058-04 | ME05058-05 | Control |
|---|---|---|---|---|---|---|
| T₂ | 2.19 ± 0.01 | 2.33 ± 0.01 | 1.71 ± 0.01 | 1.17 ± 0.01 | 1.23 ± 0.01 | 1.00 ± 0.36 |
| p-value | 0.03 | p < 0.01 | 0.13 | 0.31 | 0.19 | N/A |
| T₃ | 1.90 ± 0.01 | 1.14 ± 0.01 | 1.32 ± 0.01 | Not | 1.37 ± 0.01 | 1.00 ± 0.16 |
| | | | | determined | | |
| p-value | 0.09 | 0.30 | 0.20 | Not | 0.04 | N/A |
| | | | | determined | | |

*p-values were calculated using a Student's t-test

It is noteworthy that all T2 and T3 ME5058 events analyzed exhibited a “staygreen” phenotype, regardless of whether there was a significant increase in chlorophyll a or b levels. The T2 and T3 ME05058 events were darker green in color and had shinier rosettes than did the corresponding transgenic control plants.

There were no observable or statistically significant differences between T2 ME05058 events and control plants in germination, onset of flowering, rosette area, and general morphology/architecture. Seed yield was decreased in T2 ME05058 events relative to that of control plants. Finale® resistance for the T2 ME05058 events segregated at a 3:1 ratio of resistance:sensitivity, consistent with the presence of a single insert.

### Example 7

### Analysis of ME06485 Events

Carotenoid levels in aerial tissues from four ME06485 T2 events and four T3 events, each containing Ceres Clone 13930 (SEQ ID NO: 103), were analyzed as described in Example 2. As shown in Table 10, the β-carotene levels in T2 aerial tissue from two events of ME06485 were increased relative to the corresponding amounts in aerial tissues in transgenic control plants. For example, as shown in Table 10, β-carotene levels in aerial tissues from events ME06485-04 and ME06485-05 were increased by 57% and 150%, respectively, relative to the β-carotene levels in transgenic control plants. The standard error for this experiment was calculated based on three replicates for the experimental events and 12 replicates for the transgenic controls. Each replicate represented an extraction of pooled tissues from four individual plants. No statistically significant increases in β-carotene were noted for the T3 aerial tissues from the ME06485 events.

There were no observable or statistically significant differences between T2 ME06485 events and control plants in germination, onset of flowering, rosette area, seed yield, and general morphology/architecture.

TABLE 10
β-Carotene Increase (Fold Increase) in ME06485 T₂ and T₃ Generations

| | ME06485-01 | ME06485-03 | ME06485-04 | ME06485-05 | Control |
|---|---|---|---|---|---|
| T₂ | 1.28 ± 0.98 | 0.91 ± 0.38 | 1.57 ± 0.26 | 2.50 ± 0.50 | 1.00 ± 0.44 |
| p-value | 0.8 | 0.62 | 0.04 | 0.02 | N/A |
| T₃ | 1.36 ± 0.63 | 0.95 ± 0.87 | 1.11 ± 0.53 | 1.07 ± 0.46 | 1.00 ± 0.44 |
| p-value | 0.49 | 0.85 | 0.88 | 0.42 | N/A |

*p-values were calculated using a Student's t-test

### Example 8

### Analysis of ME01130 Events

Carotenoid levels in aerial tissues from five ME01130 T2 events and five T3 events, each containing Ceres Clone 34589 (SEQ ID NO: 94), were analyzed as described in Example 2.

As shown in Table 11, the zeaxanthin levels in T2 aerial tissue from two events of ME01130 were significantly increased relative to the corresponding amounts in aerial tissues in transgenic control plants. For example, as shown in Table 11, zeaxanthin levels in aerial tissues from T2 events ME01130-01 and ME01130-05 were increased by 27% and 55%, respectively, relative to the zeaxanthin levels in transgenic control plants. The standard error for this experiment was calculated based on three replicates for the experimental events and 12 replicates for the transgenic controls. Each replicate represented an extraction of pooled tissues from four individual plants. No statistically significant increases in zeaxanthin were noted for the T3 aerial tissues from the ME06485 events.

There were no observable or statistically significant differences between T2 ME01130 events and control plants in germination, onset of flowering, rosette area, seed yield, and general morphology/architecture.

TABLE 11
Zeaxanthin Increase (Fold-Increase) in ME01130 T₂ and T₃ Generations

| | ME01130-01 | ME01130-02 | ME01130-03 | ME01130-04 | ME01130-05 | Control |
|---|---|---|---|---|---|---|
| T₂ | 1.27 ± 0.11 | 1.04 ± 0.17 | 1.44 ± 0.25 | 1.40 ± 0.10 | 1.55 ± 0.13 | 1.00 ± 0.22 |
| p-value | 0.02 | 0.06 | 0.2 | 0.06 | p < 0.01 | N/A |
| T₃ | 1.24 ± 0.17 | 0.96 ± 0.13 | 1.25 ± 0.16 | 0.93 ± 0.12 | 1.11 ± 0.21 | 1.00 ± 0.22 |
| p-value | 0.06 | 0.88 | 0.27 | 0.85 | 0.05 | N/A |

*p-values were calculated using a Student's t-test

### Example 9

### Analysis of ME12093 Events

Carotenoid levels in aerial tissues from three ME12093 T2 events and three T3 events, each containing Ceres Clone 316638 (SEQ ID NO: 97), were analyzed as described in Example 2. As shown in Table 11, the zeaxanthin levels in T2 aerial tissue from all three T2 events of ME12093 were significantly increased relative to the corresponding amounts in aerial tissues in transgenic control plants. For example, as shown in Table 11, zeaxanthin levels in aerial tissues from T2 events 1\1E12093-02, ME12093-03 and ME12093-04 were increased by 346%, 455% and 132%, respectively, relative to the zeaxanthin levels in transgenic control plants. The standard error for this experiment was calculated based on three replicates for the experimental events and 12 replicates for the transgenic controls. Each replicate represented an extraction of pooled tissues from four individual plants. Zeaxanthin levels in aerial tissues from the T3 events ME12093-02 were increased by 42% relative to the zeaxanthin levels in transgenic control plants.

There were no observable or statistically significant differences between T2 ME12093 events and control plants in germination, onset of flowering, rosette area, seed yield, and general morphology/architecture.

TABLE 12
ζ-Carotene Increase (Fold Increase)
in ME12093 T₂ and T₃ Generations

| | ME12093-02 | ME12093-03 | ME12093-04 | Control |
|---|---|---|---|---|
| T₂ | 4.46 ± 0.60 | 5.55 ± 0.64 | 2.32 ± 0.38 | 1.00 ± 0.25 |
| p-value | p < 0.01 | p < 0.01 | p < 0.01 | N/A |
| T₃ | 1.42 ± 0.27 | 0.79 ± 0.05 | 1.09 ± 0.05 | 1.00 ± 0.25 |
| p-value | 0.05 | 0.14 | 0.7 | N/A |

*p-values were calculated using a Student's t-test

### Example 10

### Analysis of Transgenic Tomatoes for Carotenoids, Phytosterols, and Tocopherols

Transgenic tomato plants were generated using the Microtom variety as the recipient line. Each transgenic plant line contained a construct comprising a CaMV 35S promoter operably linked to either Ceres Clone 34553 (SEQ ID NO: 79); Ceres Clone 968026 (SEQ ID NO: 90); Ceres Clone 316638 (SEQ ID NO: 97); or Ceres Clone 13930 (SEQ ID NO: 103).

Explants of cotyledons from seven to nine day old seedlings were transformed using an Agrobacterium-mediated transformation method essentially as described in Park et al., J. Plant Physiol., 160:1253-1257 (2003). Transformants were selected using a bialophos resistance gene as a selectable marker and selecting on a bialophos containing medium. After selection for transformed tissues, plants were regenerated in a greenhouse and fruit tissues were analyzed for carotenoids, phytosterols, and tocopherols.

Tomato fruits were quartered, frozen in liquid nitrogen, and lyophilized for seven days. The lyophilized tissue was ground into a fine powder, and 30 mg±3 mg of ground tissue were extracted with 1.50 mL of a 4:3 mixture of ethanol and hexane containing 0.05% w/v butylatedhydroxy toluene. Sixty μL of a solution containing 1 μg/μL of trans-crocetin in ethanol was added as an internal standard. The mixture was mixed by inversion on an orbital shaker for 45 minutes at 4° C. in the dark. Care was taken not to expose the extract to heat or light. The extract was decanted into a syringe and filtered through a 0.22 micron filter into an amber LC-MS vial. The extract was analyzed for carotenoid content using a Waters 2795 Alliance system with a 996 PDA Detector, a Micromass ZMD single quadrupole mass spectrometer, and an atmospheric pressure chemical ionization probe (Waters Corp., Milford, Mass.). Separation of molecules was accomplished using a Luna C₁₈(2) 4.6×150 mm column (Phenomenex, Torrance, Calif.). Carotenoid compounds were identified based on spectral characteristics and comparison to reference standards and published retention times.

To analyze phytosterol and tocopherol contents, 30 mg±3 mg of ground tissue per sample were placed into a 2 mL Eppendorf tube, and 1.25 mL of ethyl acetate were added to the tube along with 20 μL of a solution containing 1 mg/mL of 19-OH cholesterol in ethyl acetate. The samples were incubated at 70° C. in a heat block for 30 minutes, during which time they were vortexed every five minutes for ten seconds. The samples were then centrifuged at 14,000 g for five minutes, and the extracts were transferred to a 1.5 mL autosampler vial and dried in a Savant SpeedVac for three hours using cryovac pumping. Each dried extract was resuspended in 80 μL of pyridine, sonicated to ensure complete resuspension of the crystals, and incubated for 90 minutes at 25° C. while shaking continuously. After adding 120 μL of MSTFA (Sigma-Aldrich, Saint Louis, Mo.) to each sample, the samples were incubated at 37° C. for 30 minutes and then at room temperature for 120 minutes. Each sample was analyzed for phytosterol and tocopherol content using a QP-2010 GC-MS instrument (Shimadzu Scientific Instruments, Columbia, Md.) with a Varian FactorFour™ column (30 m×0.25 mm×0.25 μm film thickness with 10 m integrated guard column; Varian, Inc., Palo Alto, Calif.). Data were analyzed using the Shimadzu GC-MS Solutions program. Phytosterols and tocopherols were identified by means of retention time standards and mass spectral libraries. Target peak areas were integrated and normalized with respect to the internal standard and the initial weight of the sample. The experimental samples were normalized with respect to the control to obtain normalized response factors. Calibration curves were used for absolute quantitation. Results of the analyses of tomato fruit for carotenoid, phytosterol, and/or tocopherol contents are presented in Table 13.

TABLE 13
Modulation of carotenoid, phytosterol, and/or tocopherol levels
in tomatoes transformed with carotenoid-modulating polypeptides

| | | # of Events | |
|---|---|---|---|
| cDNA | Clone | Modulated/# of | Fold-increase or Fold- |
| ID | ID | Events Tested | decrease¹ |
| 23369236 | 34589 | 6/8 | Decrease in carotenoids³ |
| 23419225 | 968026 | 3/10 | 2-4X increase in ζ-carotene |
| | | 1/10 | Decrease in lycopene and |
| | | | carotenes⁵ |
| 23767585 | 316638 | 2/3 | 2X increase in β-carotene |
| | | 1/3 | 2X increase in lycopene |
| 23369276 | 13930 | 6/9 | 2-7X increase in all |
| | | | tocopherols⁶ |
| | | 2/9 | 2X increase in phytosterols⁴ |
| | | 2/9 | 2X increase in triterpenes² |
| | | 1/9 | 2X increase in β-amyrin |
| | | 1/9 | 2X increase in stigmasterol |
| | | 1/9 | 15X increase in squalene |
| | | 1/9 | Decrease in triterpenes² and |
| | | | phytosterols⁴ |

¹Fold-increase or decrease relative to wild-type tomato fruit at the four weeks post-breaker stage.
²Triterpenes include α-amyrin, β-amyrin, lupeol, and cycloartenol.
³Carotenoids include lycopene, β-carotene, δ-carotene, and ζ-carotene.
⁴Phytosterols include three major sterol forms: campesterol, sitosterol, and stigmasterol.
⁵Carotenes include β- and δ-carotene.
⁶Tocopherols include α-, β-, δ-, and γ-tocopherol.

### Example 11

### Determination of Functional Homolog and/or Orthologue Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having BLAST sequence identity of 80% or greater to the query polypeptide and an alignment length of 85% or greater along the shorter sequence in the alignment. The query polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO: 80, SEQ ID NO: 91, SEQ ID NO: 104, SEQ ID NO: 95, SEQ ID NO: 89, SEQ ID NO: 110, and SEQ ID NO: 98 are shown in FIGS. 1-5, 7 and 8, respectively.

### Example 12

### Determination of Functional Homologs by HMM

Hidden Markov Models (HMMs) were generated by the program HMMER 2.3.2. To generate each HMM, the default HMMER 2.3.2 program parameters, configured for glocal alignments, were used. An HMM was generated using the sequences shown in FIG. 1 as input. These sequences were fitted to the model and the HMM bit score for each sequence is shown in the Sequence Listing. Additional sequences were fitted to the model, and the HMM bit scores for any such additional sequences are shown in the Sequence Listing. The results indicate that these additional sequences are functional homologs of SEQ ID NO:80. The procedure above was repeated for each of FIGS. 2-9, using the sequences shown in that Figure as input to generate an HMM. The bit score for each sequence is shown in the Sequence Listing and/or in Tables 14-22. Additional sequences were fitted to certain HMMs, and the HMM bit scores for such additional sequences are shown in the Sequence Listing and/or in Tables 14-22. The results indicate that these additional sequences are functional homologs of the sequences used to generate that HMM.

TABLE 14
Bit Scores From HMM Based on Sequences in FIG. 1

| | | SEQ ID | HMM bit |
|---|---|---|---|
| Designation | Species | NO: | score |
| Ceres CLONE ID no. 34553 | Arabidopsis thaliana | 80 | 726.3 |
| Ceres CLONE ID no. 473423 | Glycine max | 81 | 703.4 |
| Ceres CLONE ID no. 614555 | Glycine max | 82 | 565.4 |
| Ceres CLONE ID no. 1329993 | Triticum aestivum | 83 | 413 |
| Ceres CLONE ID no. 258825 | Zea mays | 84 | 504.4 |
| Public GI no. 34903896 | Oryza sativa subsp. japonica | 85 | 490.9 |
| Public GI no. 57900348 | Oryza sativa subsp. japonica | 86 | 461.6 |
| Public GI no. 50906641 | Oryza sativa subsp. japonica | 87 | 571.1 |
| Ceres Annot ID no. 1534144 | Populus balsamifera subsp. | 185 | 671.3 |
| | trichocarpa | | |
| Ceres Annot ID no. 1441679 | Populus balsamifera subsp. | 187 | 498.9 |
| | trichocarpa | | |
| Ceres Annot ID no. 1480659 | Populus balsamifera subsp. | 189 | 535.4 |
| | trichocarpa | | |
| Ceres Annot ID no. 1479838 | Populus balsamifera subsp. | 191 | 481.2 |
| | trichocarpa | | |
| Ceres Annot ID no. 1533308 | Populus balsamifera subsp. | 193 | 299.8 |
| | trichocarpa | | |
| Ceres CLONE ID no. 463380 | Glycine max | 195 | 713.7 |
| Public GI no. 113367174 | Glycine max | 196 | 721.7 |
| Ceres CLONE ID no. 908192 | Triticum aestivum | 198 | 553.7 |
| Public GI no. 125538797 | Oryza sativa subsp. indica | 199 | 587.7 |
| Public GI no. 115435234 | Oryza sativa subsp. japonica | 200 | 418 |
| Public GI no. 115440013 | Oryza sativa subsp. japonica | 201 | 461.6 |
| Public GI no. 125581476 | Oryza sativa subsp. japonica | 202 | 571.4 |
| Public GI no. 115445299 | Oryza sativa subsp. japonica | 203 | 571.1 |

TABLE 15
Bit Scores From HMM Based on Sequences in FIG. 2

| | | SEQ ID | HMM bit |
|---|---|---|---|
| Designation | Species | NO: | score |
| Ceres CLONE ID no. 968026 | Brassica napus | 91 | 465.3 |
| Public GI no. 28466913 | Arabidopsis thaliana | 92 | 460.1 |
| Ceres CLONE ID no. 596510 | Glycine max | 93 | 488 |
| Ceres CLONE ID no. 1832286 | Gossypium hirsutum | 341 | 314.4 |
| Ceres CLONE ID no. 1932920 | Gossypium hirsutum | 343 | 167.4 |
| Ceres ANNOT ID no. 1473516 | Populus balsamifera subsp. | 345 | 344.9 |
| | trichocarpa | | |
| Ceres ANNOT ID no. 1526929 | Populus balsamifera subsp. | 347 | 321.5 |
| | trichocarpa | | |
| Ceres ANNOT ID no. 1474764 | Populus balsamifera subsp. | 349 | 208.2 |
| | trichocarpa | | |
| Ceres ANNOT ID no. 1443270 | Populus balsamifera subsp. | 351 | 192.4 |
| | trichocarpa | | |
| Ceres ANNOT ID no. 1496190 | Populus balsamifera subsp. | 353 | 174.6 |
| | trichocarpa | | |
| Public GI no. 15242000 | Arabidopsis thaliana | 354 | 460.1 |
| Ceres CLONE ID no. 34579 | Arabidopsis thaliana | 356 | 381 |
| Public GI no. 15228338 | Arabidopsis thaliana | 357 | 373.8 |
| Public GI no. 3953599 | Arabidopsis thaliana | 358 | 373 |
| Public GI no. 3323581 | Arabidopsis thaliana | 359 | 373.4 |
| Public GI no. 3953605 | Arabidopsis thaliana | 360 | 354 |
| Public GI no. 15230202 | Arabidopsis thaliana | 361 | 207.8 |
| Ceres CLONE ID no. 1240183 | Glycine max | 363 | 469.7 |
| Ceres CLONE ID no. 775387 | Triticum aestivum | 365 | 128.5 |
| Ceres CLONE ID no. 916238 | Triticum aestivum | 367 | 113.2 |
| Public GI no. 12060388 | Zea mays | 368 | 214.3 |
| Public GI no. 90265238 | Oryza saliva subsp. indica | 369 | 118.1 |
| Public GI no. 115484121 | Oryza saliva subsp. japonica | 370 | 202.2 |
| Public GI no. 87116390 | Oryza saliva subsp. japonica | 371 | 202.1 |

TABLE 16
Bit Scores From HMM Based on Sequences in FIG. 3

| | | SEQ ID | HMM bit |
|---|---|---|---|
| Designation | Species | NO: | score |
| Ceres CLONE ID no. 13930 | Arabidopsis thaliana | 104 | 880.6 |
| Ceres ANNOT ID no. 1455046 | Populus balsamifera subsp. | 106 | 814.6 |
| | trichocarpa | | |
| Ceres ANNOT ID no. 1475265 | Populus balsamifera subsp. | 108 | 939.8 |
| | trichocarpa | | |
| Ceres CLONE ID no. 1842178 | Gossypium hirsutum | 128 | 966.9 |
| Public GI no. 18413971 | Arabidopsis thaliana | 129 | 880.6 |
| Ceres CLONE ID no. 1044646 | Glycine max | 131 | 949.1 |

TABLE 17
Bit Scores From HMM Based on Sequences in FIG. 4

| | | SEQ ID | HMM bit |
|---|---|---|---|
| Designation | Species | NO: | score |
| Ceres CLONE ID no. 34589 | Arabidopsis thaliana | 95 | 560.4 |
| Ceres CLONE ID no. | Brassica napus | 96 | 423.6 |
| 975220 | | | |
| Ceres CLONE ID no. | Gossypium hirsutum | 205 | 716.1 |
| 1973945 | | | |
| Public GI no. 13346188 | Gossypium hirsutum | 206 | 443.3 |
| Ceres ANNOT ID no. | Populus balsamifera subsp. | 208 | 720.2 |
| 1448769 | trichocarpa | | |
| Ceres ANNOT ID no. | Populus balsamifera subsp. | 210 | 732.5 |
| 1501772 | trichocarpa | | |
| Ceres ANNOT ID no. | Populus balsamifera subsp. | 212 | 626.3 |
| 1465830 | trichocarpa | | |
| Public GI no. 15221257 | Arabidopsis thaliana | 213 | 561.3 |
| Ceres CLONE ID no. | Glycine max | 215 | 702.3 |
| 1371146 | | | |
| Public GI no. 110931782 | Glycine max | 216 | 309.1 |
| Ceres CLONE ID no. | Triticum aestivum | 218 | 452.4 |
| 1020930 | | | |
| Ceres CLONE ID no. | Triticum aestivum | 220 | 436.9 |
| 764797 | | | |
| Ceres CLONE ID no. | Zea mays | 222 | 664.7 |
| 325641 | | | |
| Public GI no. 116310449 | Oryza saliva subsp. indica | 223 | 735.4 |
| Public GI no. 115458786 | Oryza saliva subsp. japonica | 224 | 733.2 |

TABLE 18
Bit Scores From HMM Based on Sequence in FIG. 5

| | | SEQ ID | HMM bit |
|---|---|---|---|
| Designation | Species | NO: | score |
| Truncated Version Ceres CLONE ID | Artificial Sequence | 159 | 526.2 |
| 1918241 | | | |
| Truncated Version Ceres annot ID | Artificial Sequence | 161 | 491.6 |
| 1454043 | | | |
| Truncated Version Ceres Clone ID | Artificial Sequence | 163 | 468.5 |
| 1938564 | | | |
| Truncated Version Ceres Annot ID | Artificial Sequence | 165 | 544.2 |
| 1464854 | | | |
| Truncated Version Ceres Annot ID | Artificial Sequence | 167 | 521.1 |
| 1511378 | | | |
| Truncated Version Ceres Annot ID | Artificial Sequence | 169 | 521.1 |
| 1458137 | | | |
| Truncated Version Public GI no. | Artificial Sequence | 170 | 525 |
| 18424254 | | | |
| Truncated Version Ceres Clone ID | Artificial Sequence | 172 | 543.7 |
| 479514 | | | |
| Truncated Version Ceres Clone ID | Artificial Sequence | 174 | 437.9 |
| 1240790 | | | |
| Truncated Version Ceres Clone ID | Artificial Sequence | 176 | 426.4 |
| 942216 | | | |
| Truncated Version Ceres Clone ID | Artificial Sequence | 178 | 406.4 |
| 987194 | | | |
| Truncated Version Ceres Clone ID | Artificial Sequence | 180 | 522.7 |
| 1780447 | | | |
| Truncated Version Ceres Clone ID | Artificial Sequence | 182 | 406.4 |
| 677852 | | | |
| Truncated Version Public GI no. | Artificial Sequence | 183 | 409.9 |
| 115452185 | | | |
| Ceres CLONE ID no. 21863 | Arabidopsis thaliana | 89 | 516.4 |

TABLE 19
Bit Scores from HMM Based on Sequences in FIG. 6

| | | SEQ ID | HMM bit |
|---|---|---|---|
| Designation | Species | NO: | score |
| Ceres CLONE ID no. | Gossypium hirsutum | 133 | 954.7 |
| 1918241 | | | |
| Ceres CLONE ID no. | Gossypium hirsutum | 135 | 639.2 |
| 1938564 | | | |
| Ceres ANNOT ID no. | Populus balsamifera subsp. | 137 | 1045 |
| 1464854 | trichocarpa | | |
| Ceres ANNOT ID no. | Populus balsamifera subsp. | 139 | 922.6 |
| 1511378 | trichocarpa | | |
| Ceres ANNOT ID no. | Populus balsamifera subsp. | 141 | 922.6 |
| 1458137 | trichocarpa | | |
| Ceres ANNOT ID no. | Populus balsamifera subsp. | 143 | 575.5 |
| 1454043 | trichocarpa | | |
| Public GI no. 18424254 | Arabidopsis thaliana | 144 | 1018.8 |
| Ceres CLONE ID no. | Glycine max | 146 | 930.1 |
| 479514 | | | |
| Ceres CLONE ID no. | Glycine max | 148 | 598.7 |
| 1240790 | | | |
| Ceres CLONE ID no. | Triticum aestivum | 150 | 666.2 |
| 942216 | | | |
| Ceres CLONE ID no. | Zea mays | 152 | 662.6 |
| 987194 | | | |
| Ceres CLONE ID no. | Triticum aestivum | 154 | 962 |
| 1780447 | | | |
| Ceres CLONE ID no. | Triticum aestivum | 156 | 664 |
| 677852 | | | |
| Public GI no. 115452185 | Oryza saliva subsp. japonica | 157 | 669.5 |

TABLE 20
Bit Scores from HMM Based on Sequences in FIG. 7

| | | SEQ ID | HMM bit |
|---|---|---|---|
| Designation | Species | NO: | score |
| Ceres CLONE ID no. | Arabidopsis thaliana | 110 | 289.4 |
| 641355 | | | |
| Ceres CLONE ID no. | Gossypium hirsutum | 292 | 174.3 |
| 1849534 | | | |
| Ceres CLONE ID no. | Gossypium hirsutum | 294 | 670.9 |
| 1926437 | | | |
| Ceres ANNOT ID no. | Populus balsamifera subsp. | 296 | 277.3 |
| 1463335 | trichocarpa | | |
| Ceres ANNOT ID no. | Populus balsamifera subsp. | 298 | 193.1 |
| 1463334 | trichocarpa | | |
| Ceres ANNOT ID no. | Populus balsamifera subsp. | 300 | 182.1 |
| 1442765 | trichocarpa | | |
| Ceres ANNOT ID no. | Populus balsamifera subsp. | 302 | 156.8 |
| 1442760 | trichocarpa | | |
| Ceres annot | Populus balsamifera subsp. | 304 | 236.1 |
| ID no. 1446840 | trichocarpa | | |
| Public GI no. 42565130 | Arabidopsis thaliana | 305 | 183.5 |
| Public GI no. 15239863 | Arabidopsis thaliana | 306 | 258.4 |
| Public GI no. 116831575 | Arabidopsis thaliana | 307 | 258.4 |
| Public GI no. 48479320 | Arabidopsis thaliana | 308 | 259.9 |
| Public GI no. 145338854 | Arabidopsis thaliana | 309 | 187.1 |
| Public GI no. 48479286 | Arabidopsis thaliana | 310 | 164.9 |
| Public GI no. 21264420 | Arabidopsis thaliana | 311 | 310.5 |
| Public GI no. 25350257 | Arabidopsis thaliana | 312 | 267.8 |
| Public GI no. 25350258 | Arabidopsis thaliana | 313 | 307.7 |
| Ceres CLONE ID no. 6042 | Arabidopsis thaliana | 315 | 307.5 |
| Public GI no. 18414897 | Arabidopsis thaliana | 316 | 180.4 |
| Ceres CLONE ID no. | Brassica napus | 318 | 459.7 |
| 965028 | | | |
| Ceres CLONE ID no. | Brassica napus | 320 | 500.3 |
| 1080500 | | | |
| Ceres CLONE ID no. | Glycine max | 322 | 285 |
| 641355 | | | |
| Ceres CLONE ID no. | Triticum aestivum | 324 | 116.9 |
| 907605 | | | |
| Ceres CLONE ID no. | Triticum aestivum | 326 | 674.4 |
| 555364 | | | |
| Ceres CLONE ID no. | Triticum aestivum | 328 | 641 |
| 569593 | | | |
| Public GI no. 125547473 | Oryza saliva subsp. indica | 329 | 144.1 |
| Public GI no. 125562586 | Oryza saliva subsp. indica | 330 | 155.3 |
| Public GI no. 31432356 | Oryza saliva subsp. japonica | 331 | 217.7 |
| Public GI no. 125574952 | Oryza saliva subsp. japonica | 332 | 217.7 |
| Public GI no. 115450749 | Oryza saliva subsp. japonica | 333 | 150.6 |
| Public GI no. 125584936 | Oryza saliva subsp. japonica | 334 | 145.3 |
| Public GI no. 115457454 | Oryza saliva subsp. japonica | 335 | 146.5 |
| Public GI no. 52076099 | Oryza saliva subsp. japonica | 336 | 152.6 |
| Public GI no. 28071302 | Oryza saliva subsp. japonica | 337 | 153.3 |
| Public GI no. 115439973 | Oryza saliva subsp. japonica | 338 | 91.6 |
| Public GI no. 57899163 | Oryza saliva subsp. japonica | 339 | 86.9 |

TABLE 21
Bit Scores from HMM Based on Sequences in FIG. 8

| | | SEQ ID | HMM bit |
|---|---|---|---|
| Designation | Species | NO: | score |
| Ceres CLONE ID no. 973476 | Brassica napus | 100 | 219.1 |
| Ceres CLONE ID no. 911175 | Triticum aestivum | 101 | 132.7 |
| Truncated Version Ceres CLONE ID | Artificial Sequence | 259 | 226.3 |
| 1929841 | | | |
| Truncated Version Ceres annot ID 1470444 | Artificial Sequence | 261 | 223.9 |
| Truncated Version Public GI no. 15237901 | Artificial Sequence | 262 | 225.6 |
| Truncated Version Ceres CLONE ID | Artificial Sequence | 264 | 225.6 |
| 26844 | | | |
| Truncated Version Public GI no. 15228464 | Artificial Sequence | 265 | 221.2 |
| Truncated Version Ceres CLONE ID | Artificial Sequence | 267 | 221.2 |
| 124385 | | | |
| Truncated Version Ceres CLONE ID | Artificial Sequence | 269 | 224.2 |
| 707855 | | | |
| Truncated Version Ceres CLONE ID | Artificial Sequence | 271 | 225.2 |
| 757222 | | | |
| Truncated Version Ceres CLONE ID | Artificial Sequence | 273 | 224.5 |
| 1545342 | | | |
| Truncated Version Ceres CLONE ID | Artificial Sequence | 275 | 224.2 |
| 275632 | | | |
| Truncated Version Ceres CLONE ID | Artificial Sequence | 277 | 215.6 |
| 306269 | | | |
| Truncated Version Ceres CLONE ID | Artificial Sequence | 279 | 202.9 |
| 295292 | | | |
| Truncated Version Ceres CLONE ID | Artificial Sequence | 281 | 224.2 |
| 1860083 | | | |
| Truncated Version Public GI no. | Artificial Sequence | 282 | 205.4 |
| 125537931 | | | |
| Truncated Version Public GI no. 41052966 | Artificial Sequence | 283 | 224.5 |
| Truncated Version Public GI no. | Artificial Sequence | 284 | 224.5 |
| 115448099 | | | |
| Truncated Version Public GI no. | Artificial Sequence | 285 | 224.2 |
| 115483682 | | | |
| Truncated Version Public GI no. 78709067 | Artificial Sequence | 286 | 224.2 |
| Truncated Version Public GI no. | Artificial Sequence | 287 | 224.2 |
| 110289664 | | | |
| Truncated Version Public GI no. | Artificial Sequence | 288 | 205.4 |
| 115443985 | | | |
| Truncated Version Public GI no. | Artificial Sequence | 289 | 205.4 |
| 125580669 | | | |
| Truncated Version Public GI no. 7248402 | Artificial Sequence | 290 | 179.7 |
| Ceres CLONE ID no. 316638 | Zea mays | 98 | 112.3 |

TABLE 22
Bit Scores from HMM Based on Sequences in FIG. 9

| | | SEQ ID | HMM bit |
|---|---|---|---|
| Designation | Species | NO: | score |
| Public GI no. 78709067 | Oryza saliva subsp. japonica | 99 | 231.5 |
| Public GI no. 1184987 | Nicotiana tabacum | 102 | 70.5 |
| Ceres CLONE ID no. 1929841 | Gossypium hirsutum | 226 | 504.5 |
| Ceres ANNOT ID no. | Populus balsamifera subsp. | 228 | 307.4 |
| 1470444 | trichocarpa | | |
| Public GI no. 15237901 | Arabidopsis thaliana | 229 | 496.7 |
| Ceres CLONE ID no. 26844 | Arabidopsis thaliana | 231 | 496.7 |
| Public GI no. 15228464 | Arabidopsis thaliana | 232 | 484.2 |
| Ceres CLONE ID no. 124385 | Arabidopsis thaliana | 234 | 484.2 |
| Ceres CLONE ID no. 707855 | Glycine max | 236 | 503.1 |
| Ceres CLONE ID no. 757222 | Triticum aestivum | 238 | 395.2 |
| Ceres CLONE ID no. 1545342 | Zea mays | 240 | 483.8 |
| Ceres CLONE ID no. 275632 | Zea mays | 242 | 481.3 |
| Ceres CLONE ID no. 306269 | Zea mays | 244 | 486.8 |
| Ceres CLONE ID no. 295292 | Zea mays | 246 | 336.3 |
| Ceres CLONE ID no. 1860083 | Panicum virgatum | 248 | 263.6 |
| Public GI no. 125537931 | Oryza sativa subsp. indica | 249 | 296.4 |
| Public GI no. 41052966 | Oryza sativa subsp. japonica | 250 | 489.9 |
| Public GI no. 115448099 | Oryza sativa subsp. japonica | 251 | 195.7 |
| Public GI no. 115483682 | Oryza sativa subsp. japonica | 252 | 474.5 |
| Public GI no. 78709066 | Oryza sativa subsp. japonica | 253 | 305.3 |
| Public GI no. 110289664 | Oryza sativa subsp. japonica | 254 | 231.5 |
| Public GI no. 115443985 | Oryza sativa subsp. japonica | 255 | 410.5 |
| Public GI no. 125580669 | Oryza sativa subsp. japonica | 256 | 364.7 |
| Public GI no. 7248402 | Oryza sativa subsp. japonica | 257 | 368.6 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of modulating the level of a carotenoid in a plant, said method comprising introducing into a plant cell an exogenous nucleic acid comprising a regulatory region operably linked to a nucleotide sequence encoding a polypeptide, wherein the HMM bit score of the amino acid sequence of said polypeptide is greater than 50, said HMM based on the amino acid sequences depicted in one of FIGS. 1-9, and wherein a tissue of a plant produced from said plant cell has a difference in the level of a carotenoid as compared to the corresponding level in tissue of a control plant that does not comprise said exogenous nucleic acid.

2. A method of modulating the level of a carotenoid in a plant, said method comprising introducing into a plant cell an exogenous nucleic acid comprising a regulatory region operably linked to a nucleotide sequence encoding a polypeptide having 80 percent or greater sequence identity to an amino acid sequence selected from the group consisting of SEQ ID NOs: 80-87, SEQ ID NO: 89, SEQ ID NOs: 91-93, SEQ ID NO: 95, SEQ ID NO: 96, SEQ ID NO: 98; SEQ ID NO: 99, SEQ ID NO: 100, SEQ ID NO:101, SEQ ID NO:102, SEQ ID NO: 104, SEQ ID NO:106, SEQ ID NO:108, SEQ ID NO: 110, SEQ ID NO: 128, SEQ ID NO: 129, SEQ ID NO: 131; SEQ ID NO: 133, SEQ ID NO:135, SEQ ID NO:137, SEQ ID NO:139, SEQ ID NO:141, SEQ ID NO:143, SEQ ID NO:144, SEQ ID NO:146, SEQ ID NO:148, SEQ ID NO:150, SEQ ID NO:152, SEQ ID NO:154, SEQ ID NO:156, SEQ ID NO:157; SEQ ID NO: 159, SEQ ID NO: 161, SEQ ID NO: 163, SEQ ID NO: 165, SEQ ID NO: 167, SEQ ID NO: 169, SEQ ID NO: 170, SEQ ID NO: 172, SEQ ID NO: 174, SEQ ID NO: 176, SEQ ID NO: 178, SEQ ID NO: SEQ ID NO: 180, SEQ ID NO: 182, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 196, SEQ ID NO: 198, SEQ ID NOs: 199-203, SEQ ID NO: 205, SEQ ID NO: 206, SEQ ID NO: 208, SEQ ID NO: 210, SEQ ID NO: 212, SEQ ID NO: 213, SEQ ID NO: 215, SEQ ID NO: 216, SEQ ID NO: 218, SEQ ID NO: 220, SEQ ID NO: 222, SEQ ID NO: 223, SEQ ID NO: 224, SEQ ID NO:226, SEQ ID NO:228, SEQ ID NO:229, SEQ ID NO:231, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NOs:248-257, SEQ ID NO:259, SEQ ID NO:261, SEQ ID NO:262, SEQ ID NO:264, SEQ ID NO:265, SEQ ID NO:267, SEQ ID NO:269, SEQ ID NO:271, SEQ ID NO:273, SEQ ID NO:275, SEQ ID NO:277, SEQ ID NO:279, SEQ ID NOs:281-290, SEQ ID NO:292, SEQ ID NO:294, SEQ ID NO:296, SEQ ID NO:298, SEQ ID NO:300, SEQ ID NO:302, SEQ ID NOs:304-313, SEQ ID NO:315, SEQ ID NO:316, SEQ ID NO:318, SEQ ID NO:320, SEQ ID NO:322, SEQ ID NO:324, SEQ ID NO:326, SEQ ID NOs:328-339, SEQ ID NO:341, SEQ ID NO:343, SEQ ID NO:345, SEQ ID NO:347, SEQ ID NO:349, SEQ ID NO:351, SEQ ID NO:353, SEQ ID NO:354, SEQ ID NOs:356-361, SEQ ID NO:363, SEQ ID NO:365, and SEQ ID NOs:367-371, wherein a tissue of a plant produced from said plant cell has a difference in the level of a carotenoid as compared to the corresponding level in tissue of a control plant that does not comprise said exogenous nucleic acid.

3. A method of producing a plant tissue, said method comprising growing a plant cell comprising an exogenous nucleic acid, said exogenous nucleic acid comprising a regulatory region operably linked to a nucleotide sequence encoding a polypeptide, wherein the HMM bit score of the amino acid sequence of said polypeptide is greater than 50, said HMM based on the amino acid sequences depicted in one of FIGS. 1-9, and wherein a tissue of a plant produced from said plant cell has a difference in the level of a carotenoid as compared to the corresponding level in tissue of a control plant that does not comprise said exogenous nucleic acid.

4. The method of claim 1, wherein said nucleotide sequence encodes a polypeptide, the HMM bit score of the amino acid sequence of said polypeptide being greater than 50, said HMM based on the amino acid sequences depicted in FIG. 7.

5. The method of claim 4, wherein said nucleotide sequence encodes a polypeptide comprising an amino acid sequence corresponding to SEQ ID NO: 110.

6. A method of modulating the level of a carotenoid in a plant, said method comprising introducing into a plant cell an exogenous nucleic acid comprising a regulatory region operably linked to a nucleotide sequence having 80 percent or greater sequence identity to a nucleotide sequence selected from the group consisting of SEQ ID NO: 94, SEQ ID NO: 103, SEQ ID NO: 107, SEQ ID NO: 109, SEQ ID NO: 127, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 145, SEQ ID NO: 147, SEQ ID NO: 149, SEQ ID NO: 151, SEQ ID NO: 153, SEQ ID NO: 155, SEQ ID NO: 158, SEQ ID NO: 160, SEQ ID NO: 162, SEQ ID NO: 164, SEQ ID NO: 166, SEQ ID NO: 168, SEQ ID NO: 171, SEQ ID NO: 173, SEQ ID NO: 175, SEQ ID NO: 177, SEQ ID NO: 179, SEQ ID NO: 181, SEQ ID NO: 184, SEQ ID NO: 186, SEQ ID NO: 188, SEQ ID NO: 190, SEQ ID NO: 192, SEQ ID NO: 194, SEQ ID NO: 197, SEQ ID NO: 204, SEQ ID NO: 207, SEQ ID NO: 209, SEQ ID NO: 211, SEQ ID NO: 214, SEQ ID NO: 217, SEQ ID NO: 219, SEQ ID NO: 221, SEQ ID NO: 225, SEQ ID NO: 227, SEQ ID NO: 230, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 258, SEQ ID NO: 260, SEQ ID NO: 263, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 314, SEQ ID NO: 317, SEQ ID NO: 319, SEQ ID NO: 321, SEQ ID NO: 323, SEQ ID NO: 325, SEQ ID NO: 327, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 355, SEQ ID NO: 362, SEQ ID NO: 364, SEQ ID NO: 366, SEQ ID NO: 372, SEQ ID NO: 374, SEQ ID NO: 375, SEQ ID NO: 376, SEQ ID NO: 377, SEQ ID NO: 378, SEQ ID NO: 379, wherein a tissue of a plant produced from said plant cell has a difference in the level of a carotenoid as compared to the corresponding level in tissue of a control plant that does not comprise said nucleic acid.

7. A method of producing a plant tissue, said method comprising growing a plant cell comprising an exogenous nucleic acid, said exogenous nucleic acid comprising a regulatory region operably linked to a nucleotide sequence having 80 percent or greater sequence identity to a nucleotide sequence selected from the group consisting of SEQ ID NO: 94, SEQ ID NO: 103, SEQ ID NO: 107, SEQ ID NO: 109, SEQ ID NO: 127, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 145, SEQ ID NO: 147, SEQ ID NO: 149, SEQ ID NO: 151, SEQ ID NO: 153, SEQ ID NO: 155, SEQ ID NO: 158, SEQ ID NO: 160, SEQ ID NO: 162, SEQ ID NO: 164, SEQ ID NO: 166, SEQ ID NO: 168, SEQ ID NO: 171, SEQ ID NO: 173, SEQ ID NO: 175, SEQ ID NO: 177, SEQ ID NO: 179, SEQ ID NO: 181, SEQ ID NO: 184, SEQ ID NO: 186, SEQ ID NO: 188, SEQ ID NO: 190, SEQ ID NO: 192, SEQ ID NO: 194, SEQ ID NO: 197, SEQ ID NO: 204, SEQ ID NO: 207, SEQ ID NO: 209, SEQ ID NO: 211, SEQ ID NO: 214, SEQ ID NO: 217, SEQ ID NO: 219, SEQ ID NO: 221, SEQ ID NO: 225, SEQ ID NO: 227, SEQ ID NO: 230, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 258, SEQ ID NO: 260, SEQ ID NO: 263, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 314, SEQ ID NO: 317, SEQ ID NO: 319, SEQ ID NO: 321, SEQ ID NO: 323, SEQ ID NO: 325, SEQ ID NO: 327, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 355, SEQ ID NO: 362, SEQ ID NO: 364, SEQ ID NO: 366, SEQ ID NO: 372, SEQ ID NO: 374, SEQ ID NO: 375, SEQ ID NO: 376, SEQ ID NO: 377, SEQ ID NO: 378, SEQ ID NO: 379, wherein a tissue of a plant produced from said plant cell has a difference in the level of a carotenoid as compared to the corresponding level in tissue of a control plant that does not comprise said nucleic acid.

8. The method of claim 6, wherein said nucleotide sequence encodes a polypeptide, the HMM bit score of the amino acid sequence of said polypeptide being greater than 50, said HMM based on the amino acid sequences depicted in FIG. 7.

9. The method of claim 8, wherein said nucleotide sequence encodes a polypeptide comprising the amino acid sequence set forth in SEQ ID NO: 110.

10. The method of claim 6, wherein said nucleotide sequence has a sequence identity that is 85 percent or greater.

11. The method of claim 1, wherein said nucleotide sequence encodes a polypeptide comprising an amino acid sequence selected from the group consisting of SEQ ID NO: 80, SEQ ID NO: 89, SEQ ID NO: 91, SEQ ID NO: 95, SEQ ID NO: 98, SEQ ID NO: 104, SEQ ID NO: 144 and SEQ ID NO: 240.

12. The method of claim 1, wherein said difference is an increase in the level of a carotenoid.

13. The method of claim 1, wherein said carotenoid is selected from the group consisting of phytoene, ζ-carotene, lycopene, δ-carotene, α-carotene, lutein, gamma-carotene, cis-β-carotene, trans-β-carotene, zeaxanthin, antheraxanthin, astaxanthin, bixin, capsanthin, fucoxanthin, and violaxanthin.

14-16. (canceled)

17. The method of claim 1, wherein said plant is a dicot.

18. The method of claim 17, wherein said plant is a member of the genus Lycopersicon, Lactuca, Glycine, Gossypium, or Brassica.

19. The method of claim 1, wherein said plant is a monocot.

20. The method of claim 19, wherein said plant is a member of the genus Triticum, Zea, Oryza, or Musa.

21-44. (canceled)

45. An isolated nucleic acid molecule comprising a nucleotide sequence having 95% or greater sequence identity to the nucleotide sequence selected from the group consisting of SEQ ID NO:105 and SEQ ID NO:107.

46. An isolated nucleic acid comprising a nucleotide sequence encoding a polypeptide having 80% or greater sequence identity to the amino acid sequence selected from the group consisting of SEQ ID NO:106 and SEQ ID NO:108.
